SEQUENCE LISTING (669 sequences)

SEQ ID NO: 1 - DNA, 20 bp, Homo sapiens
  source 1..20: mol_type = genomic DNA
aatcaggagcgccgcgcacg

SEQ ID NO: 2 - DNA, 20 bp, Homo sapiens
  source 1..20: mol_type = genomic DNA
ggcgctgacgaccgaaggta

SEQ ID NO: 3 - DNA, 20 bp, Homo sapiens
  source 1..20: mol_type = genomic DNA
agccgcggcgatgacgtgga

SEQ ID NO: 4 - DNA, 20 bp, Homo sapiens
  source 1..20: mol_type = genomic DNA
cgctgacgaccgaaggtagg

SEQ ID NO: 5 - DNA, 20 bp, Homo sapiens
  source 1..20: mol_type = genomic DNA
ctgtgggcgctgacgaccga

SEQ ID NO: 6 - DNA, 20 bp, Homo sapiens
  source 1..20: mol_type = genomic DNA
agaatcaggagcgccgcgca

SEQ ID NO: 7 - DNA, 20 bp, Homo sapiens
  source 1..20: mol_type = genomic DNA
ggcgctgacgaccgaaggta

SEQ ID NO: 8 - DNA, 20 bp, Homo sapiens
  source 1..20: mol_type = genomic DNA
agccgcggcgatgacgtgga

SEQ ID NO: 9 - DNA, 20 bp, Homo sapiens
  source 1..20: mol_type = genomic DNA
gggtgaccaccacctcacat

SEQ ID NO: 10 - DNA, 20 bp, Homo sapiens
  source 1..20: mol_type = genomic DNA
aactcctctcaggaagaccc

SEQ ID NO: 11 - DNA, 20 bp, Homo sapiens
  source 1..20: mol_type = genomic DNA
gggtgaccaccacctcacat

SEQ ID NO: 12 - DNA, 20 bp, Homo sapiens
  source 1..20: mol_type = genomic DNA
agaggctattaatataacat

SEQ ID NO: 13 - DNA, 20 bp, Homo sapiens
  source 1..20: mol_type = genomic DNA
gagacacaatggctccaggt

SEQ ID NO: 14 - DNA, 20 bp, Homo sapiens
  source 1..20: mol_type = genomic DNA
aatgcacttgagaggagtat

SEQ ID NO: 15 - DNA, 20 bp, Homo sapiens
  source 1..20: mol_type = genomic DNA
gctctcctaacagttacatc

SEQ ID NO: 16 - DNA, 20 bp, Homo sapiens
  source 1..20: mol_type = genomic DNA
aggagagtaccgatgtgagg

SEQ ID NO: 17 - DNA, 20 bp, Homo sapiens
  source 1..20: mol_type = genomic DNA
tgggactgtctaccagtgtg

SEQ ID NO: 18 - DNA, 20 bp, Homo sapiens
  source 1..20: mol_type = genomic DNA
aagatctgggttgtctcgca

SEQ ID NO: 19 - DNA, 20 bp, Homo sapiens
  source 1..20: mol_type = genomic DNA
atgttatattaatagcctct

SEQ ID NO: 20 - DNA, 20 bp, Homo sapiens
  source 1..20: mol_type = genomic DNA
agattgttgctggatcaagt

SEQ ID NO: 21 - DNA, 20 bp, Homo sapiens
  source 1..20: mol_type = genomic DNA
catggagacacaatggctcc

SEQ ID NO: 22 - DNA, 20 bp, Homo sapiens
  source 1..20: mol_type = genomic DNA
agagtaccgatgtgaggtgg

SEQ ID NO: 23 - DNA, 20 bp, Homo sapiens
  source 1..20: mol_type = genomic DNA
tcacgtctatgggtatcacc

SEQ ID NO: 24 - DNA, 20 bp, Homo sapiens
  source 1..20: mol_type = genomic DNA
ttaatattaacacttccctc

SEQ ID NO: 25 - DNA, 20 bp, Homo sapiens
  source 1..20: mol_type = genomic DNA
agcaggagagtaccgatgtg

SEQ ID NO: 26 - DNA, 20 bp, Homo sapiens
  source 1..20: mol_type = genomic DNA
caccaagaggcattccgacc

SEQ ID NO: 27 - DNA, 20 bp, Homo sapiens
  source 1..20: mol_type = genomic DNA
gttgtctcgcaaggcaaaga

SEQ ID NO: 28 - DNA, 20 bp, Homo sapiens
  source 1..20: mol_type = genomic DNA
caggagcgccgcgcacgtgg

SEQ ID NO: 29 - DNA, 20 bp, Homo sapiens
  source 1..20: mol_type = genomic DNA
gtaggggtaacagtaaagtt

SEQ ID NO: 30 - DNA, 20 bp, Homo sapiens
  source 1..20: mol_type = genomic DNA
taggggagacaggaaggatt

SEQ ID NO: 31 - DNA, 20 bp, Homo sapiens
  source 1..20: mol_type = genomic DNA
gtctgagaaacaaaccccta

SEQ ID NO: 32 - DNA, 20 bp, Homo sapiens
  source 1..20: mol_type = genomic DNA
gggcgctgacgaccgaaggt

SEQ ID NO: 33 - DNA, 20 bp, Homo sapiens
  source 1..20: mol_type = genomic DNA
tcagcgcccacagcagaatc

SEQ ID NO: 34 - DNA, 20 bp, Homo sapiens
  source 1..20: mol_type = genomic DNA
cacactggtagacagtccca

SEQ ID NO: 35 - DNA, 20 bp, Homo sapiens
  source 1..20: mol_type = genomic DNA
ggtggtggtcacccctctga

SEQ ID NO: 36 - DNA, 20 bp, Homo sapiens
  source 1..20: mol_type = genomic DNA
gagccattgtgtctccatgg

SEQ ID NO: 37 - DNA, 20 bp, Homo sapiens
  source 1..20: mol_type = genomic DNA
cgtcagactcttccaaaacc

SEQ ID NO: 38 - DNA, 20 bp, Homo sapiens
  source 1..20: mol_type = genomic DNA
ttgatccagcaacaatctgc

SEQ ID NO: 39 - DNA, 20 bp, Homo sapiens
  source 1..20: mol_type = genomic DNA
acagtccagcttgaagttgt

SEQ ID NO: 40 - DNA, 20 bp, Homo sapiens
  source 1..20: mol_type = genomic DNA
acttgcctgatgtaactgtt

SEQ ID NO: 41 - DNA, 20 bp, Homo sapiens
  source 1..20: mol_type = genomic DNA
actcctctcaggaagaccct

SEQ ID NO: 42 - DNA, 20 bp, Homo sapiens
  source 1..20: mol_type = genomic DNA
gattccaggcagccgcagtg

SEQ ID NO: 43 - DNA, 20 bp, Homo sapiens
  source 1..20: mol_type = genomic DNA
tgcatccgagtgttccctag

SEQ ID NO: 44 - DNA, 20 bp, Homo sapiens
  source 1..20: mol_type = genomic DNA
aactcctctcaggaagaccc

SEQ ID NO: 45 - DNA, 20 bp, Homo sapiens
  source 1..20: mol_type = genomic DNA
ctgtgggcgctgacgaccga

SEQ ID NO: 46 - DNA, 20 bp, Homo sapiens
  source 1..20: mol_type = genomic DNA
acgcatgccgtaccacacac

SEQ ID NO: 47 - DNA, 20 bp, Homo sapiens
  source 1..20: mol_type = genomic DNA
ccatccatattcttgatggt

SEQ ID NO: 48 - DNA, 20 bp, Homo sapiens
  source 1..20: mol_type = genomic DNA
tacccatagacgtgatgttg

SEQ ID NO: 49 - DNA, 20 bp, Homo sapiens
  source 1..20: mol_type = genomic DNA
actgtgccgagcagcagtca

SEQ ID NO: 50 - DNA, 20 bp, Homo sapiens
  source 1..20: mol_type = genomic DNA
cttgaagctgaactcctctc

SEQ ID NO: 51 - DNA, 20 bp, Homo sapiens
  source 1..20: mol_type = genomic DNA
aatatatgtgtgagtcaagt

SEQ ID NO: 52 - DNA, 20 bp, Homo sapiens
  source 1..20: mol_type = genomic DNA
tgaaataggccaccaatgaa

SEQ ID NO: 53 - DNA, 20 bp, Homo sapiens
  source 1..20: mol_type = genomic DNA
tggtcccaccatcaagaata

SEQ ID NO: 54 - DNA, 20 bp, Homo sapiens
  source 1..20: mol_type = genomic DNA
gtcacccctctgaaggcaca

SEQ ID NO: 55 - DNA, 20 bp, Homo sapiens
  source 1..20: mol_type = genomic DNA
atttctgaggatgtcatcac

SEQ ID NO: 56 - DNA, 20 bp, Homo sapiens
  source 1..20: mol_type = genomic DNA
tacaacactgacaaagatct

SEQ ID NO: 57 - DNA, 20 bp, Homo sapiens
  source 1..20: mol_type = genomic DNA
tgtgccatccatattcttga

SEQ ID NO: 58 - DNA, 20 bp, Homo sapiens
  source 1..20: mol_type = genomic DNA
agagtggggacgcctcactg

SEQ ID NO: 59 - DNA, 20 bp, Homo sapiens
  source 1..20: mol_type = genomic DNA
agggtaaagttgctcctcaa

SEQ ID NO: 60 - DNA, 20 bp, Homo sapiens
  source 1..20: mol_type = genomic DNA
agccgcggcgatgacgtgga

SEQ ID NO: 61 - DNA, 20 bp, Homo sapiens
  source 1..20: mol_type = genomic DNA
actcacccacaacttcaagc

SEQ ID NO: 62 - DNA, 20 bp, Homo sapiens
  source 1..20: mol_type = genomic DNA
acacactggtagacagtccc

SEQ ID NO: 63 - DNA, 20 bp, Homo sapiens
  source 1..20: mol_type = genomic DNA
ctgtctaccagtgtgtggta

SEQ ID NO: 64 - DNA, 20 bp, Homo sapiens
  source 1..20: mol_type = genomic DNA
atggtgacattgtcattcag

SEQ ID NO: 65 - DNA, 20 bp, Homo sapiens
  source 1..20: mol_type = genomic DNA
gatgtaactgttaggagagc

SEQ ID NO: 66 - DNA, 20 bp, Homo sapiens
  source 1..20: mol_type = genomic DNA
cccaccatcaagaatatgga

SEQ ID NO: 67 - DNA, 20 bp, Homo sapiens
  source 1..20: mol_type = genomic DNA
cagcctccatggagacacaa

SEQ ID NO: 68 - DNA, 20 bp, Homo sapiens
  source 1..20: mol_type = genomic DNA
ggaatccagctggaggaagc

SEQ ID NO: 69 - DNA, 20 bp, Homo sapiens
  source 1..20: mol_type = genomic DNA
acatcctcagaaatctctat

SEQ ID NO: 70 - DNA, 20 bp, Homo sapiens
  source 1..20: mol_type = genomic DNA
gctgcctggaatccagctgg

SEQ ID NO: 71 - DNA, 20 bp, Homo sapiens
  source 1..20: mol_type = genomic DNA
actttaccctgactgctgct

SEQ ID NO: 72 - DNA, 20 bp, Homo sapiens
  source 1..20: mol_type = genomic DNA
taggggtaacagtaaagttg

SEQ ID NO: 73 - DNA, 20 bp, Homo sapiens
  source 1..20: mol_type = genomic DNA
tatggtgacattgtcattca

SEQ ID NO: 74 - DNA, 20 bp, Homo sapiens
  source 1..20: mol_type = genomic DNA
gtcaagtgggttctacccag

SEQ ID NO: 75 - DNA, 20 bp, Homo sapiens
  source 1..20: mol_type = genomic DNA
agtcccagggtcttcctgag

SEQ ID NO: 76 - DNA, 20 bp, Homo sapiens
  source 1..20: mol_type = genomic DNA
ggcgctgacgaccgaaggta

SEQ ID NO: 77 - DNA, 20 bp, Homo sapiens
  source 1..20: mol_type = genomic DNA
ggtcacccctctgaaggcac

SEQ ID NO: 78 - DNA, 20 bp, Homo sapiens
  source 1..20: mol_type = genomic DNA
cccgagtgcaaaagaatatg

SEQ ID NO: 79 - DNA, 20 bp, Homo sapiens
  source 1..20: mol_type = genomic DNA
gcggctgcctggaatccagc

SEQ ID NO: 80 - DNA, 20 bp, Homo sapiens
  source 1..20: mol_type = genomic DNA
tgaaagtagagatgatggca

SEQ ID NO: 81 - DNA, 20 bp, Homo sapiens
  source 1..20: mol_type = genomic DNA
tgttccctgtgccttcagag

SEQ ID NO: 82 - DNA, 20 bp, Homo sapiens
  source 1..20: mol_type = genomic DNA
acccctcaacatcacgtcta

SEQ ID NO: 83 - DNA, 20 bp, Homo sapiens
  source 1..20: mol_type = genomic DNA
gaaagtagagatgatggcag

SEQ ID NO: 84 - DNA, 20 bp, Homo sapiens
  source 1..20: mol_type = genomic DNA
actgttccctgtgccttcag

SEQ ID NO: 85 - DNA, 20 bp, Homo sapiens
  source 1..20: mol_type = genomic DNA
atattaacacttccctcagg

SEQ ID NO: 86 - DNA, 20 bp, Homo sapiens
  source 1..20: mol_type = genomic DNA
ccatcctgcagctggtaaga

SEQ ID NO: 87 - DNA, 17 bp, Homo sapiens
  source 1..17: mol_type = genomic DNA
gtagagatgatggcagg

SEQ ID NO: 88 - DNA, 20 bp, Homo sapiens
  source 1..20: mol_type = genomic DNA
acaggagggcaaatggtccg

SEQ ID NO: 89 - DNA, 20 bp, Homo sapiens
  source 1..20: mol_type = genomic DNA
tgatctgaaagtagagatga

SEQ ID NO: 90 - DNA, 20 bp, Homo sapiens
  source 1..20: mol_type = genomic DNA
gcttcctccagctggattcc

SEQ ID NO: 91 - DNA, 20 bp, Homo sapiens
  source 1..20: mol_type = genomic DNA
tatgggatggggaaacttct

SEQ ID NO: 92 - DNA, 20 bp, Homo sapiens
  source 1..20: mol_type = genomic DNA
aagtagagatgatggcaggg

SEQ ID NO: 93 - DNA, 20 bp, Homo sapiens
  source 1..20: mol_type = genomic DNA
tccccactcttcagcctcca

SEQ ID NO: 94 - DNA, 20 bp, Homo sapiens
  source 1..20: mol_type = genomic DNA
tgtttcagaatgcacttgag

SEQ ID NO: 95 - DNA, 20 bp, Homo sapiens
  source 1..20: mol_type = genomic DNA
tccatggaggctgaagagtg

SEQ ID NO: 96 - DNA, 20 bp, Homo sapiens
  source 1..20: mol_type = genomic DNA
atggccgtcttaccagctgc

SEQ ID NO: 97 - DNA, 20 bp, Homo sapiens
  source 1..20: mol_type = genomic DNA
ccatcccatagagatttctg

SEQ ID NO: 98 - DNA, 20 bp, Homo sapiens
  source 1..20: mol_type = genomic DNA
tctgcagacaggagggcaaa

SEQ ID NO: 99 - DNA, 20 bp, Homo sapiens
  source 1..20: mol_type = genomic DNA
ggtaaagttgctcctcaagg

SEQ ID NO: 100 - DNA, 20 bp, Homo sapiens
  source 1..20: mol_type = genomic DNA
atatggtgacattgtcattc

SEQ ID NO: 101 - DNA, 20 bp, Homo sapiens
  source 1..20: mol_type = genomic DNA
ctgttccctgtgccttcaga

SEQ ID NO: 102 - DNA, 20 bp, Homo sapiens
  source 1..20: mol_type = genomic DNA
aggggagacaggaaggattg

SEQ ID NO: 103 - DNA, 20 bp, Homo sapiens
  source 1..20: mol_type = genomic DNA
ggcttgcacataaggaacct

SEQ ID NO: 104 - DNA, 20 bp, Homo sapiens
  source 1..20: mol_type = genomic DNA
gctggaggaggttcccagat

SEQ ID NO: 105 - DNA, 20 bp, Homo sapiens
  source 1..20: mol_type = genomic DNA
aagcctccccatcctgcagc

SEQ ID NO: 106 - DNA, 20 bp, Homo sapiens
  source 1..20: mol_type = genomic DNA
ttaccagctgcaggatgggg

SEQ ID NO: 107 - DNA, 20 bp, Homo sapiens
  source 1..20: mol_type = genomic DNA
ctccatggaggctgaagagt

SEQ ID NO: 108 - DNA, 20 bp, Homo sapiens
  source 1..20: mol_type = genomic DNA
acccatagacgtgatgttga

SEQ ID NO: 109 - DNA, 20 bp, Homo sapiens
  source 1..20: mol_type = genomic DNA
cgtcttaccagctgcaggat

SEQ ID NO: 110 - DNA, 20 bp, Homo sapiens
  source 1..20: mol_type = genomic DNA
agtctgagaaacaaacccct

SEQ ID NO: 111 - DNA, 20 bp, Homo sapiens
  source 1..20: mol_type = genomic DNA
aagtgcggcgcgggtggggc

SEQ ID NO: 112 - DNA, 20 bp, Homo sapiens
  source 1..20: mol_type = genomic DNA
ggtggggctggtcacgcgcc

SEQ ID NO: 113 - DNA, 20 bp, Homo sapiens
  source 1..20: mol_type = genomic DNA
ggttcgcactgcaggaagcc

SEQ ID NO: 114 - DNA, 20 bp, Homo sapiens
  source 1..20: mol_type = genomic DNA
atttcccaggttcgcactgc

SEQ ID NO: 115 - DNA, 20 bp, Homo sapiens
  source 1..20: mol_type = genomic DNA
ggcttcctgcagtgcgaacc

SEQ ID NO: 116 - DNA, 20 bp, Homo sapiens
  source 1..20: mol_type = genomic DNA
gcttcctgcagtgcgaacct

SEQ ID NO: 117 - DNA, 20 bp, Homo sapiens
  source 1..20: mol_type = genomic DNA
cgaagtcgcctggatttccc

SEQ ID NO: 118 - DNA, 20 bp, Homo sapiens
  source 1..20: mol_type = genomic DNA
agtgcgaacctgggaaatcc

SEQ ID NO: 119 - DNA, 20 bp, Homo sapiens
  source 1..20: mol_type = genomic DNA
tcggcaactgcgaagtcgcc

SEQ ID NO: 120 - DNA, 20 bp, Homo sapiens
  source 1..20: mol_type = genomic DNA
ggcgacttcgcagttgccga

SEQ ID NO: 121 - DNA, 20 bp, Homo sapiens
  source 1..20: mol_type = genomic DNA
gcgacttcgcagttgccgaa

SEQ ID NO: 122 - DNA, 20 bp, Homo sapiens
  source 1..20: mol_type = genomic DNA
cccacttcttcagatccctt

SEQ ID NO: 123 - DNA, 20 bp, Homo sapiens
  source 1..20: mol_type = genomic DNA
gccgaagggatctgaagaag

SEQ ID NO: 124 - DNA, 20 bp, Homo sapiens
  source 1..20: mol_type = genomic DNA
ccgaagggatctgaagaagt

SEQ ID NO: 125 - DNA, 20 bp, Homo sapiens
  source 1..20: mol_type = genomic DNA
agtgggatgtgcaaaagcgc

SEQ ID NO: 126 - DNA, 20 bp, Homo sapiens
  source 1..20: mol_type = genomic DNA
ggatgtgcaaaagcgccggc

SEQ ID NO: 127 - DNA, 20 bp, Homo sapiens
  source 1..20: mol_type = genomic DNA
acacagccgggatttccagc

SEQ ID NO: 128 - DNA, 17 bp, Homo sapiens
  source 1..17: mol_type = genomic DNA
gcgccggctggaaatcc

SEQ ID NO: 129 - DNA, 20 bp, Homo sapiens
  source 1..20: mol_type = genomic DNA
gagttgacggagacacagcc

SEQ ID NO: 130 - DNA, 20 bp, Homo sapiens
  source 1..20: mol_type = genomic DNA
agagttgacggagacacagc

SEQ ID NO: 131 - DNA, 20 bp, Homo sapiens
  source 1..20: mol_type = genomic DNA
tctgttgcgtaaagagttga

SEQ ID NO: 132 - DNA, 20 bp, Homo sapiens
  source 1..20: mol_type = genomic DNA
gtcaactctttacgcaacag

SEQ ID NO: 133 - DNA, 20 bp, Homo sapiens
  source 1..20: mol_type = genomic DNA
cggtagaaaccaagggcagg

SEQ ID NO: 134 - DNA, 20 bp, Homo sapiens
  source 1..20: mol_type = genomic DNA
ccggtagaaaccaagggcag

SEQ ID NO: 135 - DNA, 20 bp, Homo sapiens
  source 1..20: mol_type = genomic DNA
cagaggtctccccctgccct

SEQ ID NO: 136 - DNA, 20 bp, Homo sapiens
  source 1..20: mol_type = genomic DNA
cccggtagaaaccaagggca

SEQ ID NO: 137 - DNA, 20 bp, Homo sapiens
  source 1..20: mol_type = genomic DNA
gcccggtagaaaccaagggc

SEQ ID NO: 138 - DNA, 20 bp, Homo sapiens
  source 1..20: mol_type = genomic DNA
ggcggcccggtagaaaccaa

SEQ ID NO: 139 - DNA, 20 bp, Homo sapiens
  source 1..20: mol_type = genomic DNA
aggcggcccggtagaaacca

SEQ ID NO: 140 - DNA, 20 bp, Homo sapiens
  source 1..20: mol_type = genomic DNA
cccctgcccttggtttctac

SEQ ID NO: 141 - DNA, 20 bp, Homo sapiens
  source 1..20: mol_type = genomic DNA
ccctgcccttggtttctacc

SEQ ID NO: 142 - DNA, 20 bp, Homo sapiens
  source 1..20: mol_type = genomic DNA
ccgagtgggagcaggcggcc

SEQ ID NO: 143 - DNA, 20 bp, Homo sapiens
  source 1..20: mol_type = genomic DNA
tttcgccgagtgggagcagg

SEQ ID NO: 144 - DNA, 20 bp, Homo sapiens
  source 1..20: mol_type = genomic DNA
ttttttcgccgagtgggagc

SEQ ID NO: 145 - DNA, 20 bp, Homo sapiens
  source 1..20: mol_type = genomic DNA
ccgggccgcctgctcccact

SEQ ID NO: 146 - DNA, 20 bp, Homo sapiens
  source 1..20: mol_type = genomic DNA
tgtaatttttttcgccgagt

SEQ ID NO: 147 - DNA, 20 bp, Homo sapiens
  source 1..20: mol_type = genomic DNA
gtgtaatttttttcgccgag

SEQ ID NO: 148 - DNA, 17 bp, Homo sapiens
  source 1..17: mol_type = genomic DNA
ttacacaacagcagccg

SEQ ID NO: 149 - DNA, 20 bp, Homo sapiens
  source 1..20: mol_type = genomic DNA
agccctccacgtcatcgccg

SEQ ID NO: 150 - DNA, 20 bp, Homo sapiens
  source 1..20: mol_type = genomic DNA
cagcagccgcggcgatgacg

SEQ ID NO: 151 - DNA, 20 bp, Homo sapiens
  source 1..20: mol_type = genomic DNA
cagccgcggcgatgacgtgg

SEQ ID NO: 152 - DNA, 20 bp, Homo sapiens
  source 1..20: mol_type = genomic DNA
agccgcggcgatgacgtgga

SEQ ID NO: 153 - DNA, 20 bp, Homo sapiens
  source 1..20: mol_type = genomic DNA
gagcgccgcgcacgtggagg

SEQ ID NO: 154 - DNA, 20 bp, Homo sapiens
  source 1..20: mol_type = genomic DNA
caggagcgccgcgcacgtgg

SEQ ID NO: 155 - DNA, 20 bp, Homo sapiens
  source 1..20: mol_type = genomic DNA
ggctgccgcctccacgtgcg

SEQ ID NO: 156 - DNA, 20 bp, Homo sapiens
  source 1..20: mol_type = genomic DNA
aatcaggagcgccgcgcacg

SEQ ID NO: 157 - DNA, 20 bp, Homo sapiens
  source 1..20: mol_type = genomic DNA
tcagcgcccacagcagaatc

SEQ ID NO: 158 - DNA, 20 bp, Homo sapiens
  source 1..20: mol_type = genomic DNA
cggcgctcctgattctgctg

SEQ ID NO: 159 - DNA, 20 bp, Homo sapiens
  source 1..20: mol_type = genomic DNA
ggcgctcctgattctgctgt

SEQ ID NO: 160 - DNA, 20 bp, Homo sapiens
  source 1..20: mol_type = genomic DNA
tgatctgaaagtagagatga

SEQ ID NO: 161 - DNA, 20 bp, Homo sapiens
  source 1..20: mol_type = genomic DNA
ctgaaagtagagatgatggc

SEQ ID NO: 162 - DNA, 20 bp, Homo sapiens
  source 1..20: mol_type = genomic DNA
tgaaagtagagatgatggca

SEQ ID NO: 163 - DNA, 20 bp, Homo sapiens
  source 1..20: mol_type = genomic DNA
gaaagtagagatgatggcag

SEQ ID NO: 164 - DNA, 17 bp, Homo sapiens
  source 1..17: mol_type = genomic DNA
gtagagatgatggcagg

SEQ ID NO: 165 - DNA, 20 bp, Homo sapiens
  source 1..20: mol_type = genomic DNA
aagtagagatgatggcaggg

SEQ ID NO: 166 - DNA, 20 bp, Homo sapiens
  source 1..20: mol_type = genomic DNA
atggtgacattgtcattcag

SEQ ID NO: 167 - DNA, 20 bp, Homo sapiens
  source 1..20: mol_type = genomic DNA
tatggtgacattgtcattca

SEQ ID NO: 168 - DNA, 20 bp, Homo sapiens
  source 1..20: mol_type = genomic DNA
atatggtgacattgtcattc

SEQ ID NO: 169 - DNA, 20 bp, Homo sapiens
  source 1..20: mol_type = genomic DNA
ataaaagatattgcagaata

SEQ ID NO: 170 - DNA, 20 bp, Homo sapiens
  source 1..20: mol_type = genomic DNA
agacgtgatgttgaggggtt

SEQ ID NO: 171 - DNA, 20 bp, Homo sapiens
  source 1..20: mol_type = genomic DNA
tagacgtgatgttgaggggt

SEQ ID NO: 172 - DNA, 20 bp, Homo sapiens
  source 1..20: mol_type = genomic DNA
cccatagacgtgatgttgag

SEQ ID NO: 173 - DNA, 20 bp, Homo sapiens
  source 1..20: mol_type = genomic DNA
acccatagacgtgatgttga

SEQ ID NO: 174 - DNA, 20 bp, Homo sapiens
  source 1..20: mol_type = genomic DNA
tacccatagacgtgatgttg

SEQ ID NO: 175 - DNA, 20 bp, Homo sapiens
  source 1..20: mol_type = genomic DNA
acccctcaacatcacgtcta

SEQ ID NO: 176 - DNA, 20 bp, Homo sapiens
  source 1..20: mol_type = genomic DNA
cccctcaacatcacgtctat

SEQ ID NO: 177 - DNA, 20 bp, Homo sapiens
  source 1..20: mol_type = genomic DNA
tcacgtctatgggtatcacc

SEQ ID NO: 178 - DNA, 20 bp, Homo sapiens
  source 1..20: mol_type = genomic DNA
ctatgggtatcacctggttt

SEQ ID NO: 179 - DNA, 20 bp, Homo sapiens
  source 1..20: mol_type = genomic DNA
cgtcagactcttccaaaacc

SEQ ID NO: 180 - DNA, 20 bp, Homo sapiens
  source 1..20: mol_type = genomic DNA
gtcaaagtctttgaattttt

SEQ ID NO: 181 - DNA, 20 bp, Homo sapiens
  source 1..20: mol_type = genomic DNA
attttttggagatcaccaag

SEQ ID NO: 182 - DNA, 20 bp, Homo sapiens
  source 1..20: mol_type = genomic DNA
ctccaggtcggaatgcctct

SEQ ID NO: 183 - DNA, 20 bp, Homo sapiens
  source 1..20: mol_type = genomic DNA
caccaagaggcattccgacc

SEQ ID NO: 184 - DNA, 20 bp, Homo sapiens
  source 1..20: mol_type = genomic DNA
gagacacaatggctccaggt

SEQ ID NO: 185 - DNA, 20 bp, Homo sapiens
  source 1..20: mol_type = genomic DNA
catggagacacaatggctcc

SEQ ID NO: 186 - DNA, 20 bp, Homo sapiens
  source 1..20: mol_type = genomic DNA
cagcctccatggagacacaa

SEQ ID NO: 187 - DNA, 20 bp, Homo sapiens
  source 1..20: mol_type = genomic DNA
ctggagccattgtgtctcca

SEQ ID NO: 188 - DNA, 20 bp, Homo sapiens
  source 1..20: mol_type = genomic DNA
gagccattgtgtctccatgg

SEQ ID NO: 189 - DNA, 20 bp, Homo sapiens
  source 1..20: mol_type = genomic DNA
tccccactcttcagcctcca

SEQ ID NO: 190 - DNA, 20 bp, Homo sapiens
  source 1..20: mol_type = genomic DNA
tctccatggaggctgaagag

SEQ ID NO: 191 - DNA, 20 bp, Homo sapiens
  source 1..20: mol_type = genomic DNA
ctccatggaggctgaagagt

SEQ ID NO: 192 - DNA, 20 bp, Homo sapiens
  source 1..20: mol_type = genomic DNA
tccatggaggctgaagagtg

SEQ ID NO: 193 - DNA, 20 bp, Homo sapiens
  source 1..20: mol_type = genomic DNA
agagtggggacgcctcactg

SEQ ID NO: 194 - DNA, 20 bp, Homo sapiens
  source 1..20: mol_type = genomic DNA
gattccaggcagccgcagtg

SEQ ID NO: 195 - DNA, 20 bp, Homo sapiens
  source 1..20: mol_type = genomic DNA
gacgcctcactgcggctgcc

SEQ ID NO: 196 - DNA, 20 bp, Homo sapiens
  source 1..20: mol_type = genomic DNA
gcttcctccagctggattcc

SEQ ID NO: 197 - DNA, 20 bp, Homo sapiens
  source 1..20: mol_type = genomic DNA
gcggctgcctggaatccagc

SEQ ID NO: 198 - DNA, 20 bp, Homo sapiens
  source 1..20: mol_type = genomic DNA
gctgcctggaatccagctgg

SEQ ID NO: 199 - DNA, 20 bp, Homo sapiens
  source 1..20: mol_type = genomic DNA
actctcctgcttcctccagc

SEQ ID NO: 200 - DNA, 20 bp, Homo sapiens
  source 1..20: mol_type = genomic DNA
ggaatccagctggaggaagc

SEQ ID NO: 201 - DNA, 20 bp, Homo sapiens
  source 1..20: mol_type = genomic DNA
agcaggagagtaccgatgtg

SEQ ID NO: 202 - DNA, 20 bp, Homo sapiens
  source 1..20: mol_type = genomic DNA
gggtgaccaccacctcacat

SEQ ID NO: 203 - DNA, 20 bp, Homo sapiens
  source 1..20: mol_type = genomic DNA
aggagagtaccgatgtgagg

SEQ ID NO: 204 - DNA, 20 bp, Homo sapiens
  source 1..20: mol_type = genomic DNA
agagtaccgatgtgaggtgg

SEQ ID NO: 205 - DNA, 20 bp, Homo sapiens
  source 1..20: mol_type = genomic DNA
ggtggtggtcacccctctga

SEQ ID NO: 206 - DNA, 20 bp, Homo sapiens
  source 1..20: mol_type = genomic DNA
tgttccctgtgccttcagag

SEQ ID NO: 207 - DNA, 20 bp, Homo sapiens
  source 1..20: mol_type = genomic DNA
ctgttccctgtgccttcaga

SEQ ID NO: 208 - DNA, 20 bp, Homo sapiens
  source 1..20: mol_type = genomic DNA
actgttccctgtgccttcag

SEQ ID NO: 209 - DNA, 20 bp, Homo sapiens
  source 1..20: mol_type = genomic DNA
ggtcacccctctgaaggcac

SEQ ID NO: 210 - DNA, 20 bp, Homo sapiens
  source 1..20: mol_type = genomic DNA
gtcacccctctgaaggcaca

SEQ ID NO: 211 - DNA, 20 bp, Homo sapiens
  source 1..20: mol_type = genomic DNA
aacagtccagcttgaagttg

SEQ ID NO: 212 - DNA, 20 bp, Homo sapiens
  source 1..20: mol_type = genomic DNA
cagcaacaatctgctggctg

SEQ ID NO: 213 - DNA, 20 bp, Homo sapiens
  source 1..20: mol_type = genomic DNA
ccagcaacaatctgctggct

SEQ ID NO: 214 - DNA, 20 bp, Homo sapiens
  source 1..20: mol_type = genomic DNA
tccagcaacaatctgctggc

SEQ ID NO: 215 - DNA, 20 bp, Homo sapiens
  source 1..20: mol_type = genomic DNA
ttgatccagcaacaatctgc

SEQ ID NO: 216 - DNA, 20 bp, Homo sapiens
  source 1..20: mol_type = genomic DNA
cccagccagcagattgttgc

SEQ ID NO: 217 - DNA, 20 bp, Homo sapiens
  source 1..20: mol_type = genomic DNA
cagattgttgctggatcaag

SEQ ID NO: 218 - DNA, 20 bp, Homo sapiens
  source 1..20: mol_type = genomic DNA
agattgttgctggatcaagt

SEQ ID NO: 219 - DNA, 17 bp, Homo sapiens
  source 1..17: mol_type = genomic DNA
tatatgtgtgagtcaag

SEQ ID NO: 220 - DNA, 20 bp, Homo sapiens
  source 1..20: mol_type = genomic DNA
aatatatgtgtgagtcaagt

SEQ ID NO: 221 - DNA, 20 bp, Homo sapiens
  source 1..20: mol_type = genomic DNA
gtcaagtgggttctacccag

SEQ ID NO: 222 - DNA, 20 bp, Homo sapiens
  source 1..20: mol_type = genomic DNA
atgttatattaatagcctct

SEQ ID NO: 223 - DNA, 20 bp, Homo sapiens
  source 1..20: mol_type = genomic DNA
catgttatattaatagcctc

SEQ ID NO: 224 - DNA, 20 bp, Homo sapiens
  source 1..20: mol_type = genomic DNA
cagaggctattaatataaca

SEQ ID NO: 225 - DNA, 20 bp, Homo sapiens
  source 1..20: mol_type = genomic DNA
agaggctattaatataacat

SEQ ID NO: 226 - DNA, 20 bp, Homo sapiens
  source 1..20: mol_type = genomic DNA
tatgggatggggaaacttct

SEQ ID NO: 227 - DNA, 20 bp, Homo sapiens
  source 1..20: mol_type = genomic DNA
ctatgggatggggaaacttc

SEQ ID NO: 228 - DNA, 20 bp, Homo sapiens
  source 1..20: mol_type = genomic DNA
tcagaaatctctatgggatg

SEQ ID NO: 229 - DNA, 20 bp, Homo sapiens
  source 1..20: mol_type = genomic DNA
ctcagaaatctctatgggat

SEQ ID NO: 230 - DNA, 20 bp, Homo sapiens
  source 1..20: mol_type = genomic DNA
cctcagaaatctctatggga

SEQ ID NO: 231 - DNA, 20 bp, Homo sapiens
  source 1..20: mol_type = genomic DNA
acatcctcagaaatctctat

SEQ ID NO: 232 - DNA, 20 bp, Homo sapiens
  source 1..20: mol_type = genomic DNA
gacatcctcagaaatctcta

SEQ ID NO: 233 - DNA, 20 bp, Homo sapiens
  source 1..20: mol_type = genomic DNA
ccatcccatagagatttctg

SEQ ID NO: 234 - DNA, 20 bp, Homo sapiens
  source 1..20: mol_type = genomic DNA
atttctgaggatgtcatcac

SEQ ID NO: 235 - DNA, 20 bp, Homo sapiens
  source 1..20: mol_type = genomic DNA
ccatccatattcttgatggt

SEQ ID NO: 236 - DNA, 20 bp, Homo sapiens
  source 1..20: mol_type = genomic DNA
gccatccatattcttgatgg

SEQ ID NO: 237 - DNA, 20 bp, Homo sapiens
  source 1..20: mol_type = genomic DNA
tgtgccatccatattcttga

SEQ ID NO: 238 - DNA, 20 bp, Homo sapiens
  source 1..20: mol_type = genomic DNA
tggtcccaccatcaagaata

SEQ ID NO: 239 - DNA, 20 bp, Homo sapiens
  source 1..20: mol_type = genomic DNA
cccaccatcaagaatatgga

SEQ ID NO: 240 - DNA, 20 bp, Homo sapiens
  source 1..20: mol_type = genomic DNA
cttgaagctgaactcctctc

SEQ ID NO: 241 - DNA, 20 bp, Homo sapiens
  source 1..20: mol_type = genomic DNA
agtcccagggtcttcctgag

SEQ ID NO: 242 - DNA, 20 bp, Homo sapiens
  source 1..20: mol_type = genomic DNA
aactcctctcaggaagaccc

SEQ ID NO: 243 - DNA, 20 bp, Homo sapiens
  source 1..20: mol_type = genomic DNA
actcctctcaggaagaccct

SEQ ID NO: 244 - DNA, 20 bp, Homo sapiens
  source 1..20: mol_type = genomic DNA
cacactggtagacagtccca

SEQ ID NO: 245 - DNA, 20 bp, Homo sapiens
  source 1..20: mol_type = genomic DNA
acacactggtagacagtccc

SEQ ID NO: 246 - DNA, 20 bp, Homo sapiens
  source 1..20: mol_type = genomic DNA
tgggactgtctaccagtgtg

SEQ ID NO: 247 - DNA, 20 bp, Homo sapiens
  source 1..20: mol_type = genomic DNA
acgcatgccgtaccacacac

SEQ ID NO: 248 - DNA, 20 bp, Homo sapiens
  source 1..20: mol_type = genomic DNA
ctgtctaccagtgtgtggta

SEQ ID NO: 249 - DNA, 20 bp, Homo sapiens
  source 1..20: mol_type = genomic DNA
gctcctcaagggggtatgca

SEQ ID NO: 250 - DNA, 20 bp, Homo sapiens
  source 1..20: mol_type = genomic DNA
cgtccttgcatacccccttg

SEQ ID NO: 251 - DNA, 20 bp, Homo sapiens
  source 1..20: mol_type = genomic DNA
ggtaaagttgctcctcaagg

SEQ ID NO: 252 - DNA, 20 bp, Homo sapiens
  source 1..20: mol_type = genomic DNA
gggtaaagttgctcctcaag

SEQ ID NO: 253 - DNA, 20 bp, Homo sapiens
  source 1..20: mol_type = genomic DNA
agggtaaagttgctcctcaa

SEQ ID NO: 254 - DNA, 20 bp, Homo sapiens
  source 1..20: mol_type = genomic DNA
cagggtaaagttgctcctca

SEQ ID NO: 255 - DNA, 20 bp, Homo sapiens
  source 1..20: mol_type = genomic DNA
actgtgccgagcagcagtca

SEQ ID NO: 256 - DNA, 20 bp, Homo sapiens
  source 1..20: mol_type = genomic DNA
gactgtgccgagcagcagtc

SEQ ID NO: 257 - DNA, 20 bp, Homo sapiens
  source 1..20: mol_type = genomic DNA
actttaccctgactgctgct

SEQ ID NO: 258 - DNA, 20 bp, Homo sapiens
  source 1..20: mol_type = genomic DNA
cagataatttttccattcat

SEQ ID NO: 259 - DNA, 20 bp, Homo sapiens
  source 1..20: mol_type = genomic DNA
tgaaataggccaccaatgaa

SEQ ID NO: 260 - DNA, 20 bp, Homo sapiens
  source 1..20: mol_type = genomic DNA
ataatttttccattcattgg

SEQ ID NO: 261 - DNA, 20 bp, Homo sapiens
  source 1..20: mol_type = genomic DNA
ccaacaccaatgaatgaaat

SEQ ID NO: 262 - DNA, 20 bp, Homo sapiens
  source 1..20: mol_type = genomic DNA
tggtggcctatttcattcat

SEQ ID NO: 263 - DNA, 20 bp, Homo sapiens
  source 1..20: mol_type = genomic DNA
cctatttcattcattggtgt

SEQ ID NO: 264 - DNA, 20 bp, Homo sapiens
  source 1..20: mol_type = genomic DNA
ttcattcattggtgttggac

SEQ ID NO: 265 - DNA, 20 bp, Homo sapiens
  source 1..20: mol_type = genomic DNA
tattaattgttttgattcct

SEQ ID NO: 266 - DNA, 20 bp, Homo sapiens
  source 1..20: mol_type = genomic DNA
aatgcacttgagaggagtat

SEQ ID NO: 267 - DNA, 20 bp, Homo sapiens
  source 1..20: mol_type = genomic DNA
tgtttcagaatgcacttgag

SEQ ID NO: 268 - DNA, 20 bp, Homo sapiens
  source 1..20: mol_type = genomic DNA
tcaagtgcattctgaaacac

SEQ ID NO: 269 - DNA, 20 bp, Homo sapiens
  source 1..20: mol_type = genomic DNA
cttcagagtctgagtgtcaa

SEQ ID NO: 270 - DNA, 20 bp, Homo sapiens
  source 1..20: mol_type = genomic DNA
cccgagtgcaaaagaatatg

SEQ ID NO: 271 - DNA, 20 bp, Homo sapiens
  source 1..20: mol_type = genomic DNA
acctcatattcttttgcact

SEQ ID NO: 272 - DNA, 20 bp, Homo sapiens
  source 1..20: mol_type = genomic DNA
cctcatattcttttgcactc

SEQ ID NO: 273 - DNA, 20 bp, Homo sapiens
  source 1..20: mol_type = genomic DNA
tattcttttgcactcgggca

SEQ ID NO: 274 - DNA, 20 bp, Homo sapiens
  source 1..20: mol_type = genomic DNA
ccatcctgcagctggtaaga

SEQ ID NO: 275 - DNA, 20 bp, Homo sapiens
  source 1..20: mol_type = genomic DNA
atggccgtcttaccagctgc

SEQ ID NO: 276 - DNA, 20 bp, Homo sapiens
  source 1..20: mol_type = genomic DNA
aagcctccccatcctgcagc

SEQ ID NO: 277 - DNA, 20 bp, Homo sapiens
  source 1..20: mol_type = genomic DNA
ccgtcttaccagctgcagga

SEQ ID NO: 278 - DNA, 20 bp, Homo sapiens
  source 1..20: mol_type = genomic DNA
cgtcttaccagctgcaggat

SEQ ID NO: 279 - DNA, 20 bp, Homo sapiens
  source 1..20: mol_type = genomic DNA
gtcttaccagctgcaggatg

SEQ ID NO: 280 - DNA, 20 bp, Homo sapiens
  source 1..20: mol_type = genomic DNA
ttaccagctgcaggatgggg

SEQ ID NO: 281 - DNA, 20 bp, Homo sapiens
  source 1..20: mol_type = genomic DNA
agctgcaggatggggaggct

SEQ ID NO: 282 - DNA, 20 bp, Homo sapiens
  source 1..20: mol_type = genomic DNA
tggggaggcttggcctcctg

SEQ ID NO: 283 - DNA, 20 bp, Homo sapiens
  source 1..20: mol_type = genomic DNA
ggggaggcttggcctcctga

SEQ ID NO: 284 - DNA, 20 bp, Homo sapiens
  source 1..20: mol_type = genomic DNA
atattaacacttccctcagg

SEQ ID NO: 285 - DNA, 20 bp, Homo sapiens
  source 1..20: mol_type = genomic DNA
ttaatattaacacttccctc

SEQ ID NO: 286 - DNA, 20 bp, Homo sapiens
  source 1..20: mol_type = genomic DNA
actagatgttttctgcagac

SEQ ID NO: 287 - DNA, 20 bp, Homo sapiens
  source 1..20: mol_type = genomic DNA
agatgttttctgcagacagg

SEQ ID NO: 288 - DNA, 20 bp, Homo sapiens
  source 1..20: mol_type = genomic DNA
gatgttttctgcagacagga

SEQ ID NO: 289 - DNA, 20 bp, Homo sapiens
  source 1..20: mol_type = genomic DNA
tctgcagacaggagggcaaa

SEQ ID NO: 290 - DNA, 20 bp, Homo sapiens
  source 1..20: mol_type = genomic DNA
acaggagggcaaatggtccg

SEQ ID NO: 291 - DNA, 20 bp, Homo sapiens
  source 1..20: mol_type = genomic DNA
ggcttgcacataaggaacct

SEQ ID NO: 292 - DNA, 20 bp, Homo sapiens
  source 1..20: mol_type = genomic DNA
gcaaagaaggcttgcacata

SEQ ID NO: 293 - DNA, 20 bp, Homo sapiens
  source 1..20: mol_type = genomic DNA
gttgtctcgcaaggcaaaga

SEQ ID NO: 294 - DNA, 20 bp, Homo sapiens
  source 1..20: mol_type = genomic DNA
aagatctgggttgtctcgca

SEQ ID NO: 295 - DNA, 20 bp, Homo sapiens
  source 1..20: mol_type = genomic DNA
tacaacactgacaaagatct

SEQ ID NO: 296 - DNA, 20 bp, Homo sapiens
  source 1..20: mol_type = genomic DNA
ctacaacactgacaaagatc

SEQ ID NO: 297 - DNA, 20 bp, Homo sapiens
  source 1..20: mol_type = genomic DNA
atgtaactgttaggagagca

SEQ ID NO: 298 - DNA, 20 bp, Homo sapiens
  source 1..20: mol_type = genomic DNA
gatgtaactgttaggagagc

SEQ ID NO: 299 - DNA, 20 bp, Homo sapiens
  source 1..20: mol_type = genomic DNA
acttgcctgatgtaactgtt

SEQ ID NO: 300 - DNA, 20 bp, Homo sapiens
  source 1..20: mol_type = genomic DNA
gctctcctaacagttacatc

SEQ ID NO: 301 - DNA, 20 bp, Homo sapiens
  source 1..20: mol_type = genomic DNA
ctttgtggaattatcatcta

SEQ ID NO: 302 - DNA, 20 bp, Homo sapiens
  source 1..20: mol_type = genomic DNA
ggtttgtttctcagactttg

SEQ ID NO: 303 - DNA, 20 bp, Homo sapiens
  source 1..20: mol_type = genomic DNA
agtctgagaaacaaacccct

SEQ ID NO: 304 - DNA, 20 bp, Homo sapiens
  source 1..20: mol_type = genomic DNA
gtctgagaaacaaaccccta

SEQ ID NO: 305 - DNA, 20 bp, Homo sapiens
  source 1..20: mol_type = genomic DNA
tgcatccgagtgttccctag

SEQ ID NO: 306 - DNA, 20 bp, Homo sapiens
  source 1..20: mol_type = genomic DNA
ctgcatccgagtgttcccta

SEQ ID NO: 307 - DNA, 20 bp, Homo sapiens
  source 1..20: mol_type = genomic DNA
actgcatccgagtgttccct

SEQ ID NO: 308 - DNA, 20 bp, Homo sapiens
  source 1..20: mol_type = genomic DNA
acaaacccctagggaacact

SEQ ID NO: 309 - DNA, 20 bp, Homo sapiens
  source 1..20: mol_type = genomic DNA
ggaacactcggatgcagttc

SEQ ID NO: 310 - DNA, 20 bp, Homo sapiens
  source 1..20: mol_type = genomic DNA
acaggaaggattggggcatc

SEQ ID NO: 311 - DNA, 20 bp, Homo sapiens
  source 1..20: mol_type = genomic DNA
aggggagacaggaaggattg

SEQ ID NO: 312 - DNA, 20 bp, Homo sapiens
  source 1..20: mol_type = genomic DNA
taggggagacaggaaggatt

SEQ ID NO: 313 - DNA, 20 bp, Homo sapiens
  source 1..20: mol_type = genomic DNA
ataggggagacaggaaggat

SEQ ID NO: 314 - DNA, 20 bp, Homo sapiens
  source 1..20: mol_type = genomic DNA
cccagataggggagacagga

SEQ ID NO: 315 - DNA, 20 bp, Homo sapiens
  source 1..20: mol_type = genomic DNA
ggttcccagataggggagac

SEQ ID NO: 316 - DNA, 20 bp, Homo sapiens
  source 1..20: mol_type = genomic DNA
tccttcctgtctcccctatc

SEQ ID NO: 317 - DNA, 20 bp, Homo sapiens
  source 1..20: mol_type = genomic DNA
ccttcctgtctcccctatct

SEQ ID NO: 318 - DNA, 20 bp, Homo sapiens
  source 1..20: mol_type = genomic DNA
tggaggaggttcccagatag

SEQ ID NO: 319 - DNA, 20 bp, Homo sapiens
  source 1..20: mol_type = genomic DNA
ctggaggaggttcccagata

SEQ ID NO: 320 - DNA, 20 bp, Homo sapiens
  source 1..20: mol_type = genomic DNA
gctggaggaggttcccagat

SEQ ID NO: 321 - DNA, 20 bp, Homo sapiens
  source 1..20: mol_type = genomic DNA
gttgatgttgtggctggagg

SEQ ID NO: 322 - DNA, 20 bp, Homo sapiens
  source 1..20: mol_type = genomic DNA
gttgttgatgttgtggctgg

SEQ ID NO: 323 - DNA, 20 bp, Homo sapiens
  source 1..20: mol_type = genomic DNA
ggagttgttgatgttgtggc

SEQ ID NO: 324 - DNA, 20 bp, Homo sapiens
  source 1..20: mol_type = genomic DNA
aactggagttgttgatgttg

SEQ ID NO: 325 - DNA, 20 bp, Homo sapiens
  source 1..20: mol_type = genomic DNA
gggggttgggaggatagaac

SEQ ID NO: 326 - DNA, 20 bp, Homo sapiens
  source 1..20: mol_type = genomic DNA
cagtaaagttgggggttggg

SEQ ID NO: 327 - DNA, 20 bp, Homo sapiens
  source 1..20: mol_type = genomic DNA
taacagtaaagttgggggtt

SEQ ID NO: 328 - DNA, 20 bp, Homo sapiens
  source 1..20: mol_type = genomic DNA
gtaacagtaaagttgggggt

SEQ ID NO: 329 - DNA, 20 bp, Homo sapiens
  source 1..20: mol_type = genomic DNA
aggggtaacagtaaagttgg

SEQ ID NO: 330 - DNA, 20 bp, Homo sapiens
  source 1..20: mol_type = genomic DNA
taggggtaacagtaaagttg

SEQ ID NO: 331 - DNA, 20 bp, Homo sapiens
  source 1..20: mol_type = genomic DNA
gtaggggtaacagtaaagtt

SEQ ID NO: 332 - DNA, 20 bp, Homo sapiens
  source 1..20: mol_type = genomic DNA
tgtaggggtaacagtaaagt

SEQ ID NO: 333 - DNA, 20 bp, Homo sapiens
  source 1..20: mol_type = genomic DNA
cgcgcctggcttcctgcagt

SEQ ID NO: 334 - DNA, 20 bp, Homo sapiens
  source 1..20: mol_type = genomic DNA
atttcccaggttcgcactgc

SEQ ID NO: 335 - DNA, 20 bp, Homo sapiens
  source 1..20: mol_type = genomic DNA
gatttcccaggttcgcactg

SEQ ID NO: 336 - DNA, 20 bp, Homo sapiens
  source 1..20: mol_type = genomic DNA
tggcttcctgcagtgcgaac

SEQ ID NO: 337 - DNA, 20 bp, Homo sapiens
  source 1..20: mol_type = genomic DNA
ggcttcctgcagtgcgaacc

SEQ ID NO: 338 - DNA, 20 bp, Homo sapiens
  source 1..20: mol_type = genomic DNA
gcttcctgcagtgcgaacct

SEQ ID NO: 339 - DNA, 20 bp, Homo sapiens
  source 1..20: mol_type = genomic DNA
ttcggcaactgcgaagtcgc

SEQ ID NO: 340 - DNA, 20 bp, Homo sapiens
  source 1..20: mol_type = genomic DNA
tccaggcgacttcgcagttg

SEQ ID NO: 341 - DNA, 20 bp, Homo sapiens
  source 1..20: mol_type = genomic DNA
ttcagatcccttcggcaact

SEQ ID NO: 342 - DNA, 20 bp, Homo sapiens
  source 1..20: mol_type = genomic DNA
aggcgacttcgcagttgccg

SEQ ID NO: 343 - DNA, 20 bp, Homo sapiens
  source 1..20: mol_type = genomic DNA
ggcgacttcgcagttgccga

SEQ ID NO: 344 - DNA, 20 bp, Homo sapiens
  source 1..20: mol_type = genomic DNA
ttcgcagttgccgaagggat

SEQ ID NO: 345 - DNA, 20 bp, Homo sapiens
  source 1..20: mol_type = genomic DNA
gcagttgccgaagggatctg

SEQ ID NO: 346 - DNA, 20 bp, Homo sapiens
  source 1..20: mol_type = genomic DNA
tgccgaagggatctgaagaa

SEQ ID NO: 347 - DNA, 20 bp, Homo sapiens
  source 1..20: mol_type = genomic DNA
gccgaagggatctgaagaag

SEQ ID NO: 348 - DNA, 20 bp, Homo sapiens
  source 1..20: mol_type = genomic DNA
gggatgtgcaaaagcgccgg

SEQ ID NO: 349 - DNA, 20 bp, Homo sapiens
  source 1..20: mol_type = genomic DNA
ggatgtgcaaaagcgccggc

SEQ ID NO: 350 - DNA, 20 bp, Homo sapiens
  source 1..20: mol_type = genomic DNA
agagttgacggagacacagc

SEQ ID NO: 351 - DNA, 20 bp, Homo sapiens
  source 1..20: mol_type = genomic DNA
aagagttgacggagacacag

SEQ ID NO: 352 - DNA, 20 bp, Homo sapiens
  source 1..20: mol_type = genomic DNA
tctgttgcgtaaagagttga

SEQ ID NO: 353 - DNA, 20 bp, Homo sapiens
  source 1..20: mol_type = genomic DNA
ctctgttgcgtaaagagttg

SEQ ID NO: 354 - DNA, 20 bp, Homo sapiens
  source 1..20: mol_type = genomic DNA
cgcgcctggcttcctgcagt

SEQ ID NO: 355 - DNA, 20 bp, Homo sapiens
  source 1..20: mol_type = genomic DNA
atttcccaggttcgcactgc

SEQ ID NO: 356 - DNA, 20 bp, Homo sapiens
  source 1..20: mol_type = genomic DNA
gatttcccaggttcgcactg

SEQ ID NO: 357 - DNA, 20 bp, Homo sapiens
  source 1..20: mol_type = genomic DNA
tggcttcctgcagtgcgaac

SEQ ID NO: 358 - DNA, 20 bp, Homo sapiens
  source 1..20: mol_type = genomic DNA
ggcttcctgcagtgcgaacc

SEQ ID NO: 359 - DNA, 20 bp, Homo sapiens
  source 1..20: mol_type = genomic DNA
gcttcctgcagtgcgaacct

SEQ ID NO: 360 - DNA, 20 bp, Homo sapiens
  source 1..20: mol_type = genomic DNA
ttcggcaactgcgaagtcgc

SEQ ID NO: 361 - DNA, 20 bp, Homo sapiens
  source 1..20: mol_type = genomic DNA
tccaggcgacttcgcagttg

SEQ ID NO: 362 - DNA, 20 bp, Homo sapiens
  source 1..20: mol_type = genomic DNA
ttcagatcccttcggcaact

SEQ ID NO: 363 - DNA, 20 bp, Homo sapiens
  source 1..20: mol_type = genomic DNA
aggcgacttcgcagttgccg

SEQ ID NO: 364 - DNA, 20 bp, Homo sapiens
  source 1..20: mol_type = genomic DNA
ggcgacttcgcagttgccga

SEQ ID NO: 365 - DNA, 20 bp, Homo sapiens
  source 1..20: mol_type = genomic DNA
ttcgcagttgccgaagggat

SEQ ID NO: 366 - DNA, 20 bp, Homo sapiens
  source 1..20: mol_type = genomic DNA
gcagttgccgaagggatctg

SEQ ID NO: 367 - DNA, 20 bp, Homo sapiens
  source 1..20: mol_type = genomic DNA
tgccgaagggatctgaagaa

SEQ ID NO: 368 - DNA, 20 bp, Homo sapiens
  source 1..20: mol_type = genomic DNA
gccgaagggatctgaagaag

SEQ ID NO: 369 - DNA, 20 bp, Homo sapiens
  source 1..20: mol_type = genomic DNA
gggatgtgcaaaagcgccgg

SEQ ID NO: 370 - DNA, 20 bp, Homo sapiens
  source 1..20: mol_type = genomic DNA
ggatgtgcaaaagcgccggc

SEQ ID NO: 371 - DNA, 20 bp, Homo sapiens
  source 1..20: mol_type = genomic DNA
agagttgacggagacacagc

SEQ ID NO: 372 - DNA, 20 bp, Homo sapiens
  source 1..20: mol_type = genomic DNA
aagagttgacggagacacag

SEQ ID NO: 373 - DNA, 20 bp, Homo sapiens
  source 1..20: mol_type = genomic DNA
tctgttgcgtaaagagttga

SEQ ID NO: 374 - DNA, 20 bp, Homo sapiens
  source 1..20: mol_type = genomic DNA
ctctgttgcgtaaagagttg

SEQ ID NO: 375 - DNA, 20 bp, Homo sapiens
  source 1..20: mol_type = genomic DNA
cgcgcctggcttcctgcagt

SEQ ID NO: 376 - DNA, 20 bp, Homo sapiens
  source 1..20: mol_type = genomic DNA
atttcccaggttcgcactgc

SEQ ID NO: 377 - DNA, 20 bp, Homo sapiens
  source 1..20: mol_type = genomic DNA
gatttcccaggttcgcactg

SEQ ID NO: 378 - DNA, 20 bp, Homo sapiens
  source 1..20: mol_type = genomic DNA
tggcttcctgcagtgcgaac

SEQ ID NO: 379 - DNA, 20 bp, Homo sapiens
  source 1..20: mol_type = genomic DNA
ggcttcctgcagtgcgaacc

SEQ ID NO: 380 - DNA, 20 bp, Homo sapiens
  source 1..20: mol_type = genomic DNA
gcttcctgcagtgcgaacct

SEQ ID NO: 381 - DNA, 20 bp, Homo sapiens
  source 1..20: mol_type = genomic DNA
ttcggcaactgcgaagtcgc

SEQ ID NO: 382 - DNA, 20 bp, Homo sapiens
  source 1..20: mol_type = genomic DNA
tccaggcgacttcgcagttg

SEQ ID NO: 383 - DNA, 20 bp, Homo sapiens
  source 1..20: mol_type = genomic DNA
ttcagatcccttcggcaact

SEQ ID NO: 384 - DNA, 20 bp, Homo sapiens
  source 1..20: mol_type = genomic DNA
aggcgacttcgcagttgccg

SEQ ID NO: 385 - DNA, 20 bp, Homo sapiens
  source 1..20: mol_type = genomic DNA
ggcgacttcgcagttgccga

SEQ ID NO: 386 - DNA, 20 bp, Homo sapiens
  source 1..20: mol_type = genomic DNA
ttcgcagttgccgaagggat

SEQ ID NO: 387 - DNA, 20 bp, Homo sapiens
  source 1..20: mol_type = genomic DNA
gcagttgccgaagggatctg

SEQ ID NO: 388 - DNA, 20 bp, Homo sapiens
  source 1..20: mol_type = genomic DNA
tgccgaagggatctgaagaa

SEQ ID NO: 389 - DNA, 20 bp, Homo sapiens
  source 1..20: mol_type = genomic DNA
gccgaagggatctgaagaag

SEQ ID NO: 390 - DNA, 20 bp, Homo sapiens
  source 1..20: mol_type = genomic DNA
gggatgtgcaaaagcgccgg

SEQ ID NO: 391 - DNA, 20 bp, Homo sapiens
  source 1..20: mol_type = genomic DNA
ggatgtgcaaaagcgccggc

SEQ ID NO: 392 - DNA, 20 bp, Homo sapiens
  source 1..20: mol_type = genomic DNA
agagttgacggagacacagc

SEQ ID NO: 393 - DNA, 20 bp, Homo sapiens
  source 1..20: mol_type = genomic DNA
aagagttgacggagacacag

SEQ ID NO: 394 - DNA, 20 bp, Homo sapiens
  source 1..20: mol_type = genomic DNA
tctgttgcgtaaagagttga

SEQ ID NO: 395 - DNA, 20 bp, Homo sapiens
  source 1..20: mol_type = genomic DNA
ctctgttgcgtaaagagttg

SEQ ID NO: 396 - DNA, 20 bp, Homo sapiens
  source 1..20: mol_type = genomic DNA
cgcgcctggcttcctgcagt

SEQ ID NO: 397 - DNA, 20 bp, Homo sapiens
  source 1..20: mol_type = genomic DNA
atttcccaggttcgcactgc

SEQ ID NO: 398 - DNA, 20 bp, Homo sapiens
  source 1..20: mol_type = genomic DNA
gatttcccaggttcgcactg

SEQ ID NO: 399 - DNA, 20 bp, Homo sapiens
  source 1..20: mol_type = genomic DNA
tggcttcctgcagtgcgaac

SEQ ID NO: 400 - DNA, 20 bp, Homo sapiens
  source 1..20: mol_type = genomic DNA
ggcttcctgcagtgcgaacc

SEQ ID NO: 401 - DNA, 20 bp, Homo sapiens
  source 1..20: mol_type = genomic DNA
gcttcctgcagtgcgaacct

SEQ ID NO: 402 - DNA, 20 bp, Homo sapiens
  source 1..20: mol_type = genomic DNA
ttcggcaactgcgaagtcgc

SEQ ID NO: 403 - DNA, 20 bp, Homo sapiens
  source 1..20: mol_type = genomic DNA
tccaggcgacttcgcagttg

SEQ ID NO: 404 - DNA, 20 bp, Homo sapiens
  source 1..20: mol_type = genomic DNA
ttcagatcccttcggcaact

SEQ ID NO: 405 - DNA, 20 bp, Homo sapiens
  source 1..20: mol_type = genomic DNA
aggcgacttcgcagttgccg

SEQ ID NO: 406 - DNA, 20 bp, Homo sapiens
  source 1..20: mol_type = genomic DNA
ggcgacttcgcagttgccga

SEQ ID NO: 407 - DNA, 20 bp, Homo sapiens
  source 1..20: mol_type = genomic DNA
ttcgcagttgccgaagggat

SEQ ID NO: 408 - DNA, 20 bp, Homo sapiens
  source 1..20: mol_type = genomic DNA
gcagttgccgaagggatctg

SEQ ID NO: 409 - DNA, 20 bp, Homo sapiens
  source 1..20: mol_type = genomic DNA
tgccgaagggatctgaagaa

SEQ ID NO: 410 - DNA, 20 bp, Homo sapiens
  source 1..20: mol_type = genomic DNA
gccgaagggatctgaagaag

SEQ ID NO: 411 - DNA, 20 bp, Homo sapiens
  source 1..20: mol_type = genomic DNA
gggatgtgcaaaagcgccgg

SEQ ID NO: 412 - DNA, 20 bp, Homo sapiens
  source 1..20: mol_type = genomic DNA
ggatgtgcaaaagcgccggc

SEQ ID NO: 413 - DNA, 20 bp, Homo sapiens
  source 1..20: mol_type = genomic DNA
agagttgacggagacacagc

SEQ ID NO: 414 - DNA, 20 bp, Homo sapiens
  source 1..20: mol_type = genomic DNA
aagagttgacggagacacag

SEQ ID NO: 415 - DNA, 20 bp, Homo sapiens
  source 1..20: mol_type = genomic DNA
tctgttgcgtaaagagttga

SEQ ID NO: 416 - DNA, 20 bp, Homo sapiens
  source 1..20: mol_type = genomic DNA
ctctgttgcgtaaagagttg

SEQ ID NO: 417 - DNA, 20 bp, Homo sapiens
  source 1..20: mol_type = genomic DNA
cgcgcctggcttcctgcagt

SEQ ID NO: 418 - DNA, 20 bp, Homo sapiens
  source 1..20: mol_type = genomic DNA
atttcccaggttcgcactgc

SEQ ID NO: 419 - DNA, 20 bp, Homo sapiens
  source 1..20: mol_type = genomic DNA
gatttcccaggttcgcactg

SEQ ID NO: 420 - DNA, 20 bp, Homo sapiens
  source 1..20: mol_type = genomic DNA
tggcttcctgcagtgcgaac

SEQ ID NO: 421 - DNA, 20 bp, Homo sapiens
  source 1..20: mol_type = genomic DNA
ggcttcctgcagtgcgaacc

SEQ ID NO: 422 - DNA, 20 bp, Homo sapiens
  source 1..20: mol_type = genomic DNA
gcttcctgcagtgcgaacct

SEQ ID NO: 423 - DNA, 20 bp, Homo sapiens
  source 1..20: mol_type = genomic DNA
ttcggcaactgcgaagtcgc

SEQ ID NO: 424 - DNA, 20 bp, Homo sapiens
  source 1..20: mol_type = genomic DNA
tccaggcgacttcgcagttg

SEQ ID NO: 425 - DNA, 20 bp, Homo sapiens
  source 1..20: mol_type = genomic DNA
ttcagatcccttcggcaact

SEQ ID NO: 426 - DNA, 20 bp, Homo sapiens
  source 1..20: mol_type = genomic DNA
aggcgacttcgcagttgccg

SEQ ID NO: 427 - DNA, 20 bp, Homo sapiens
  source 1..20: mol_type = genomic DNA
ggcgacttcgcagttgccga

SEQ ID NO: 428 - DNA, 20 bp, Homo sapiens
  source 1..20: mol_type = genomic DNA
ttcgcagttgccgaagggat

SEQ ID NO: 429 - DNA, 20 bp, Homo sapiens
  source 1..20: mol_type = genomic DNA
gcagttgccgaagggatctg

SEQ ID NO: 430 - DNA, 20 bp, Homo sapiens
  source 1..20: mol_type = genomic DNA
tgccgaagggatctgaagaa

SEQ ID NO: 431 - DNA, 20 bp, Homo sapiens
  source 1..20: mol_type = genomic DNA
gccgaagggatctgaagaag

SEQ ID NO: 432 - DNA, 20 bp, Homo sapiens
  source 1..20: mol_type = genomic DNA
gggatgtgcaaaagcgccgg

SEQ ID NO: 433 - DNA, 20 bp, Homo sapiens
  source 1..20: mol_type = genomic DNA
ggatgtgcaaaagcgccggc

SEQ ID NO: 434 - DNA, 20 bp, Homo sapiens
  source 1..20: mol_type = genomic DNA
agagttgacggagacacagc

SEQ ID NO: 435 - DNA, 20 bp, Homo sapiens
  source 1..20: mol_type = genomic DNA
aagagttgacggagacacag

SEQ ID NO: 436 - DNA, 20 bp, Homo sapiens
  source 1..20: mol_type = genomic DNA
tctgttgcgtaaagagttga

SEQ ID NO: 437 - DNA, 20 bp, Homo sapiens
  source 1..20: mol_type = genomic DNA
ctctgttgcgtaaagagttg

SEQ ID NO: 438 - DNA, 20 bp, Homo sapiens
  source 1..20: mol_type = genomic DNA
cgcgcctggcttcctgcagt

SEQ ID NO: 439 - DNA, 20 bp, Homo sapiens
  source 1..20: mol_type = genomic DNA
atttcccaggttcgcactgc

SEQ ID NO: 440 - DNA, 20 bp, Homo sapiens
  source 1..20: mol_type = genomic DNA
gatttcccaggttcgcactg

SEQ ID NO: 441 - DNA, 20 bp, Homo sapiens
  source 1..20: mol_type = genomic DNA
tggcttcctgcagtgcgaac

SEQ ID NO: 442 - DNA, 20 bp, Homo sapiens
  source 1..20: mol_type = genomic DNA
ggcttcctgcagtgcgaacc

SEQ ID NO: 443 - DNA, 20 bp, Homo sapiens
  source 1..20: mol_type = genomic DNA
gcttcctgcagtgcgaacct

SEQ ID NO: 444 - DNA, 20 bp, Homo sapiens
  source 1..20: mol_type = genomic DNA
ttcggcaactgcgaagtcgc

SEQ ID NO: 445 - DNA, 20 bp, Homo sapiens
  source 1..20: mol_type = genomic DNA
tccaggcgacttcgcagttg

SEQ ID NO: 446 - DNA, 20 bp, Homo sapiens
  source 1..20: mol_type = genomic DNA
ttcagatcccttcggcaact

SEQ ID NO: 447 - DNA, 20 bp, Homo sapiens
  source 1..20: mol_type = genomic DNA
aggcgacttcgcagttgccg

SEQ ID NO: 448 - DNA, 20 bp, Homo sapiens
  source 1..20: mol_type = genomic DNA
ggcgacttcgcagttgccga

SEQ ID NO: 449 - DNA, 20 bp, Homo sapiens
  source 1..20: mol_type = genomic DNA
ttcgcagttgccgaagggat

SEQ ID NO: 450 - DNA, 20 bp, Homo sapiens
  source 1..20: mol_type = genomic DNA
gcagttgccgaagggatctg

SEQ ID NO: 451 - DNA, 20 bp, Homo sapiens
  source 1..20: mol_type = genomic DNA
tgccgaagggatctgaagaa

SEQ ID NO: 452 - DNA, 20 bp, Homo sapiens
  source 1..20: mol_type = genomic DNA
gccgaagggatctgaagaag

SEQ ID NO: 453 - DNA, 20 bp, Homo sapiens
  source 1..20: mol_type = genomic DNA
gggatgtgcaaaagcgccgg

SEQ ID NO: 454 - DNA, 20 bp, Homo sapiens
  source 1..20: mol_type = genomic DNA
ggatgtgcaaaagcgccggc

SEQ ID NO: 455 - DNA, 20 bp, Homo sapiens
  source 1..20: mol_type = genomic DNA
agagttgacggagacacagc

SEQ ID NO: 456 - DNA, 20 bp, Homo sapiens
  source 1..20: mol_type = genomic DNA
aagagttgacggagacacag

SEQ ID NO: 457 - DNA, 20 bp, Homo sapiens
  source 1..20: mol_type = genomic DNA
tctgttgcgtaaagagttga

SEQ ID NO: 458 - DNA, 20 bp, Homo sapiens
  source 1..20: mol_type = genomic DNA
ctctgttgcgtaaagagttg

SEQ ID NO: 459 - DNA, 20 bp, Homo sapiens
  source 1..20: mol_type = genomic DNA
cgcgcctggcttcctgcagt

SEQ ID NO: 460 - DNA, 20 bp, Homo sapiens
  source 1..20: mol_type = genomic DNA
atttcccaggttcgcactgc

SEQ ID NO: 461 - DNA, 20 bp, Homo sapiens
  source 1..20: mol_type = genomic DNA
gatttcccaggttcgcactg

SEQ ID NO: 462 - DNA, 20 bp, Homo sapiens
  source 1..20: mol_type = genomic DNA
tggcttcctgcagtgcgaac

SEQ ID NO: 463 - DNA, 20 bp, Homo sapiens
  source 1..20: mol_type = genomic DNA
ggcttcctgcagtgcgaacc

SEQ ID NO: 464 - DNA, 20 bp, Homo sapiens
  source 1..20: mol_type = genomic DNA
gcttcctgcagtgcgaacct

SEQ ID NO: 465 - DNA, 20 bp, Homo sapiens
  source 1..20: mol_type = genomic DNA
ttcggcaactgcgaagtcgc

SEQ ID NO: 466 - DNA, 20 bp, Homo sapiens
  source 1..20: mol_type = genomic DNA
tccaggcgacttcgcagttg

SEQ ID NO: 467 - DNA, 20 bp, Homo sapiens
  source 1..20: mol_type = genomic DNA
ttcagatcccttcggcaact

SEQ ID NO: 468 - DNA, 20 bp, Homo sapiens
  source 1..20: mol_type = genomic DNA
aggcgacttcgcagttgccg

SEQ ID NO: 469 - DNA, 20 bp, Homo sapiens
  source 1..20: mol_type = genomic DNA
ggcgacttcgcagttgccga

SEQ ID NO: 470 - DNA, 20 bp, Homo sapiens
  source 1..20: mol_type = genomic DNA
ttcgcagttgccgaagggat

SEQ ID NO: 471 - DNA, 20 bp, Homo sapiens
  source 1..20: mol_type = genomic DNA
gcagttgccgaagggatctg

SEQ ID NO: 472 - DNA, 20 bp, Homo sapiens
  source 1..20: mol_type = genomic DNA
tgccgaagggatctgaagaa

SEQ ID NO: 473 - DNA, 20 bp, Homo sapiens
  source 1..20: mol_type = genomic DNA
gccgaagggatctgaagaag

SEQ ID NO: 474 - DNA, 20 bp, Homo sapiens
  source 1..20: mol_type = genomic DNA
gggatgtgcaaaagcgccgg

SEQ ID NO: 475 - DNA, 20 bp, Homo sapiens
  source 1..20: mol_type = genomic DNA
ggatgtgcaaaagcgccggc

SEQ ID NO: 476 - DNA, 20 bp, Homo sapiens
  source 1..20: mol_type = genomic DNA
agagttgacggagacacagc

SEQ ID NO: 477 - DNA, 20 bp, Homo sapiens
  source 1..20: mol_type = genomic DNA
aagagttgacggagacacag

SEQ ID NO: 478 - DNA, 20 bp, Homo sapiens
  source 1..20: mol_type = genomic DNA
tctgttgcgtaaagagttga

SEQ ID NO: 479 - DNA, 20 bp, Homo sapiens
  source 1..20: mol_type = genomic DNA
ctctgttgcgtaaagagttg

SEQ ID NO: 480 - DNA, 20 bp, Homo sapiens
  source 1..20: mol_type = genomic DNA
cgcgcctggcttcctgcagt

SEQ ID NO: 481 - DNA, 20 bp, Homo sapiens
  source 1..20: mol_type = genomic DNA
atttcccaggttcgcactgc

SEQ ID NO: 482 - DNA, 20 bp, Homo sapiens
  source 1..20: mol_type = genomic DNA
gatttcccaggttcgcactg

SEQ ID NO: 483 - DNA, 20 bp, Homo sapiens
  source 1..20: mol_type = genomic DNA
tggcttcctgcagtgcgaac

SEQ ID NO: 484 - DNA, 20 bp, Homo sapiens
  source 1..20: mol_type = genomic DNA
ggcttcctgcagtgcgaacc

SEQ ID NO: 485 - DNA, 20 bp, Homo sapiens
  source 1..20: mol_type = genomic DNA
gcttcctgcagtgcgaacct

SEQ ID NO: 486 - DNA, 20 bp, Homo sapiens
  source 1..20: mol_type = genomic DNA
ttcggcaactgcgaagtcgc

SEQ ID NO: 487 - DNA, 20 bp, Homo sapiens
  source 1..20: mol_type = genomic DNA
tccaggcgacttcgcagttg

SEQ ID NO: 488 - DNA, 20 bp, Homo sapiens
  source 1..20: mol_type = genomic DNA
ttcagatcccttcggcaact

SEQ ID NO: 489 - DNA, 20 bp, Homo sapiens
  source 1..20: mol_type = genomic DNA
aggcgacttcgcagttgccg

SEQ ID NO: 490 - DNA, 20 bp, Homo sapiens
  source 1..20: mol_type = genomic DNA
ggcgacttcgcagttgccga

SEQ ID NO: 491 - DNA, 20 bp, Homo sapiens
  source 1..20: mol_type = genomic DNA
ttcgcagttgccgaagggat

SEQ ID NO: 492 - DNA, 20 bp, Homo sapiens
  source 1..20: mol_type = genomic DNA
gcagttgccgaagggatctg

SEQ ID NO: 493 - DNA, 20 bp, Homo sapiens
  source 1..20: mol_type = genomic DNA
tgccgaagggatctgaagaa

SEQ ID NO: 494 - DNA, 20 bp, Homo sapiens
  source 1..20: mol_type = genomic DNA
gccgaagggatctgaagaag

SEQ ID NO: 495 - DNA, 20 bp, Homo sapiens
  source 1..20: mol_type = genomic DNA
gggatgtgcaaaagcgccgg

SEQ ID NO: 496 - DNA, 20 bp, Homo sapiens
  source 1..20: mol_type = genomic DNA
ggatgtgcaaaagcgccggc

SEQ ID NO: 497 - DNA, 20 bp, Homo sapiens
  source 1..20: mol_type = genomic DNA
agagttgacggagacacagc

SEQ ID NO: 498 - DNA, 20 bp, Homo sapiens
  source 1..20: mol_type = genomic DNA
aagagttgacggagacacag

SEQ ID NO: 499 - DNA, 20 bp, Homo sapiens
  source 1..20: mol_type = genomic DNA
tctgttgcgtaaagagttga

SEQ ID NO: 500 - DNA, 20 bp, Homo sapiens
  source 1..20: mol_type = genomic DNA
ctctgttgcgtaaagagttg

SEQ ID NO: 501 - DNA, 20 bp, Homo sapiens
  source 1..20: mol_type = genomic DNA
cgcgcctggcttcctgcagt

SEQ ID NO: 502 - DNA, 20 bp, Homo sapiens
  source 1..20: mol_type = genomic DNA
atttcccaggttcgcactgc

SEQ ID NO: 503 - DNA, 20 bp, Homo sapiens
  source 1..20: mol_type = genomic DNA
gatttcccaggttcgcactg

SEQ ID NO: 504 - DNA, 20 bp, Homo sapiens
  source 1..20: mol_type = genomic DNA
tggcttcctgcagtgcgaac

SEQ ID NO: 505 - DNA, 20 bp, Homo sapiens
  source 1..20: mol_type = genomic DNA
ggcttcctgcagtgcgaacc

SEQ ID NO: 506 - DNA, 20 bp, Homo sapiens
  source 1..20: mol_type = genomic DNA
gcttcctgcagtgcgaacct

SEQ ID NO: 507 - DNA, 20 bp, Homo sapiens
  source 1..20: mol_type = genomic DNA
ttcggcaactgcgaagtcgc

SEQ ID NO: 508 - DNA, 20 bp, Homo sapiens
  source 1..20: mol_type = genomic DNA
tccaggcgacttcgcagttg

SEQ ID NO: 509 - DNA, 20 bp, Homo sapiens
  source 1..20: mol_type = genomic DNA
ttcagatcccttcggcaact

SEQ ID NO: 510 - DNA, 20 bp, Homo sapiens
  source 1..20: mol_type = genomic DNA
aggcgacttcgcagttgccg

SEQ ID NO: 511 - DNA, 20 bp, Homo sapiens
  source 1..20: mol_type = genomic DNA
ggcgacttcgcagttgccga

SEQ ID NO: 512 - DNA, 20 bp, Homo sapiens
  source 1..20: mol_type = genomic DNA
ttcgcagttgccgaagggat

SEQ ID NO: 513 - DNA, 20 bp, Homo sapiens
  source 1..20: mol_type = genomic DNA
gcagttgccgaagggatctg

SEQ ID NO: 514 - DNA, 20 bp, Homo sapiens
  source 1..20: mol_type = genomic DNA
tgccgaagggatctgaagaa

SEQ ID NO: 515 - DNA, 20 bp, Homo sapiens
  source 1..20: mol_type = genomic DNA
gccgaagggatctgaagaag

SEQ ID NO: 516 - DNA, 20 bp, Homo sapiens
  source 1..20: mol_type = genomic DNA
gggatgtgcaaaagcgccgg

SEQ ID NO: 517 - DNA, 20 bp, Homo sapiens
  source 1..20: mol_type = genomic DNA
ggatgtgcaaaagcgccggc

SEQ ID NO: 518 - DNA, 20 bp, Homo sapiens
  source 1..20: mol_type = genomic DNA
agagttgacggagacacagc

SEQ ID NO: 519 - DNA, 20 bp, Homo sapiens
  source 1..20: mol_type = genomic DNA
aagagttgacggagacacag

SEQ ID NO: 520 - DNA, 20 bp, Homo sapiens
  source 1..20: mol_type = genomic DNA
tctgttgcgtaaagagttga

SEQ ID NO: 521 - DNA, 20 bp, Homo sapiens
  source 1..20: mol_type = genomic DNA
ctctgttgcgtaaagagttg

SEQ ID NO: 522 - DNA, 20 bp, Homo sapiens
  source 1..20: mol_type = genomic DNA
cgcgcctggcttcctgcagt

SEQ ID NO: 523 - DNA, 20 bp, Homo sapiens
  source 1..20: mol_type = genomic DNA
atttcccaggttcgcactgc

SEQ ID NO: 524 - DNA, 20 bp, Homo sapiens
  source 1..20: mol_type = genomic DNA
gatttcccaggttcgcactg

SEQ ID NO: 525 - DNA, 20 bp, Homo sapiens
  source 1..20: mol_type = genomic DNA
tggcttcctgcagtgcgaac

SEQ ID NO: 526 - DNA, 20 bp, Homo sapiens
  source 1..20: mol_type = genomic DNA
ggcttcctgcagtgcgaacc

SEQ ID NO: 527 - DNA, 20 bp, Homo sapiens
  source 1..20: mol_type = genomic DNA
gcttcctgcagtgcgaacct

SEQ ID NO: 528 - DNA, 20 bp, Homo sapiens
  source 1..20: mol_type = genomic DNA
ttcggcaactgcgaagtcgc

SEQ ID NO: 529 - DNA, 20 bp, Homo sapiens
  source 1..20: mol_type = genomic DNA
tccaggcgacttcgcagttg

SEQ ID NO: 530 - DNA, 20 bp, Homo sapiens
  source 1..20: mol_type = genomic DNA
ttcagatcccttcggcaact

SEQ ID NO: 531 - DNA, 20 bp, Homo sapiens
  source 1..20: mol_type = genomic DNA
aggcgacttcgcagttgccg

SEQ ID NO: 532 - DNA, 20 bp, Homo sapiens
  source 1..20: mol_type = genomic DNA
ggcgacttcgcagttgccga

SEQ ID NO: 533 - DNA, 20 bp, Homo sapiens
  source 1..20: mol_type = genomic DNA
ttcgcagttgccgaagggat

SEQ ID NO: 534 - DNA, 20 bp, Homo sapiens
  source 1..20: mol_type = genomic DNA
gcagttgccgaagggatctg

SEQ ID NO: 535 - DNA, 20 bp, Homo sapiens
  source 1..20: mol_type = genomic DNA
tgccgaagggatctgaagaa

SEQ ID NO: 536 - DNA, 20 bp, Homo sapiens
  source 1..20: mol_type = genomic DNA
gccgaagggatctgaagaag

SEQ ID NO: 537 - DNA, 20 bp, Homo sapiens
  source 1..20: mol_type = genomic DNA
gggatgtgcaaaagcgccgg

SEQ ID NO: 538 - DNA, 20 bp, Homo sapiens
  source 1..20: mol_type = genomic DNA
ggatgtgcaaaagcgccggc

SEQ ID NO: 539 - DNA, 20 bp, Homo sapiens
  source 1..20: mol_type = genomic DNA
agagttgacggagacacagc

SEQ ID NO: 540 - DNA, 20 bp, Homo sapiens
  source 1..20: mol_type = genomic DNA
aagagttgacggagacacag

SEQ ID NO: 541 - DNA, 20 bp, Homo sapiens
  source 1..20: mol_type = genomic DNA
tctgttgcgtaaagagttga

SEQ ID NO: 542 - DNA, 20 bp, Homo sapiens
  source 1..20: mol_type = genomic DNA
ctctgttgcgtaaagagttg

SEQ ID NO: 543 - DNA, 20 bp, Homo sapiens
  source 1..20: mol_type = genomic DNA
cgcgcctggcttcctgcagt

SEQ ID NO: 544 - DNA, 20 bp, Homo sapiens
  source 1..20: mol_type = genomic DNA
atttcccaggttcgcactgc

SEQ ID NO: 545 - DNA, 20 bp, Homo sapiens
  source 1..20: mol_type = genomic DNA
gatttcccaggttcgcactg

SEQ ID NO: 546 - DNA, 20 bp, Homo sapiens
  source 1..20: mol_type = genomic DNA
tggcttcctgcagtgcgaac

SEQ ID NO: 547 - DNA, 20 bp, Homo sapiens
  source 1..20: mol_type = genomic DNA
ggcttcctgcagtgcgaacc

SEQ ID NO: 548 - DNA, 20 bp, Homo sapiens
  source 1..20: mol_type = genomic DNA
gcttcctgcagtgcgaacct

SEQ ID NO: 549 - DNA, 20 bp, Homo sapiens
  source 1..20: mol_type = genomic DNA
ttcggcaactgcgaagtcgc

SEQ ID NO: 550 - DNA, 20 bp, Homo sapiens
  source 1..20: mol_type = genomic DNA
tccaggcgacttcgcagttg

SEQ ID NO: 551 - DNA, 20 bp, Homo sapiens
  source 1..20: mol_type = genomic DNA
ttcagatcccttcggcaact

SEQ ID NO: 552 - DNA, 20 bp, Homo sapiens
  source 1..20: mol_type = genomic DNA
aggcgacttcgcagttgccg

SEQ ID NO: 553 - DNA, 20 bp, Homo sapiens
  source 1..20: mol_type = genomic DNA
ggcgacttcgcagttgccga

SEQ ID NO: 554 - DNA, 20 bp, Homo sapiens
  source 1..20: mol_type = genomic DNA
ttcgcagttgccgaagggat

SEQ ID NO: 555 - DNA, 20 bp, Homo sapiens
  source 1..20: mol_type = genomic DNA
gcagttgccgaagggatctg

SEQ ID NO: 556 - DNA, 20 bp, Homo sapiens
  source 1..20: mol_type = genomic DNA
tgccgaagggatctgaagaa

SEQ ID NO: 557 - DNA, 20 bp, Homo sapiens
  source 1..20: mol_type = genomic DNA
gccgaagggatctgaagaag

SEQ ID NO: 558 - DNA, 20 bp, Homo sapiens
  source 1..20: mol_type = genomic DNA
gggatgtgcaaaagcgccgg

SEQ ID NO: 559 - DNA, 20 bp, Homo sapiens
  source 1..20: mol_type = genomic DNA
ggatgtgcaaaagcgccggc

SEQ ID NO: 560 - DNA, 20 bp, Homo sapiens
  source 1..20: mol_type = genomic DNA
agagttgacggagacacagc

SEQ ID NO: 561 - DNA, 20 bp, Homo sapiens
  source 1..20: mol_type = genomic DNA
aagagttgacggagacacag

SEQ ID NO: 562 - DNA, 20 bp, Homo sapiens
  source 1..20: mol_type = genomic DNA
tctgttgcgtaaagagttga

SEQ ID NO: 563 - DNA, 20 bp, Homo sapiens
  source 1..20: mol_type = genomic DNA
ctctgttgcgtaaagagttg

SEQ ID NO: 564 - DNA, 20 bp, Homo sapiens
  source 1..20: mol_type = genomic DNA
cgcgcctggcttcctgcagt

SEQ ID NO: 565 - DNA, 20 bp, Homo sapiens
  source 1..20: mol_type = genomic DNA
atttcccaggttcgcactgc

SEQ ID NO: 566 - DNA, 20 bp, Homo sapiens
  source 1..20: mol_type = genomic DNA
gatttcccaggttcgcactg

SEQ ID NO: 567 - DNA, 20 bp, Homo sapiens
  source 1..20: mol_type = genomic DNA
tggcttcctgcagtgcgaac

SEQ ID NO: 568 - DNA, 20 bp, Homo sapiens
  source 1..20: mol_type = genomic DNA
ggcttcctgcagtgcgaacc

SEQ ID NO: 569 - DNA, 20 bp, Homo sapiens
  source 1..20: mol_type = genomic DNA
gcttcctgcagtgcgaacct

SEQ ID NO: 570 - DNA, 20 bp, Homo sapiens
  source 1..20: mol_type = genomic DNA
ttcggcaactgcgaagtcgc

SEQ ID NO: 571 - DNA, 20 bp, Homo sapiens
  source 1..20: mol_type = genomic DNA
tccaggcgacttcgcagttg

SEQ ID NO: 572 - DNA, 20 bp, Homo sapiens
  source 1..20: mol_type = genomic DNA
ttcagatcccttcggcaact

SEQ ID NO: 573 - DNA, 20 bp, Homo sapiens
  source 1..20: mol_type = genomic DNA
aggcgacttcgcagttgccg

SEQ ID NO: 574 - DNA, 20 bp, Homo sapiens
  source 1..20: mol_type = genomic DNA
ggcgacttcgcagttgccga

SEQ ID NO: 575 - DNA, 20 bp, Homo sapiens
  source 1..20: mol_type = genomic DNA
ttcgcagttgccgaagggat

SEQ ID NO: 576 - DNA, 20 bp, Homo sapiens
  source 1..20: mol_type = genomic DNA
gcagttgccgaagggatctg

SEQ ID NO: 577 - DNA, 20 bp, Homo sapiens
  source 1..20: mol_type = genomic DNA
tgccgaagggatctgaagaa

SEQ ID NO: 578 - DNA, 20 bp, Homo sapiens
  source 1..20: mol_type = genomic DNA
gccgaagggatctgaagaag

SEQ ID NO: 579 - DNA, 20 bp, Homo sapiens
  source 1..20: mol_type = genomic DNA
gggatgtgcaaaagcgccgg

SEQ ID NO: 580 - DNA, 20 bp, Homo sapiens
  source 1..20: mol_type = genomic DNA
ggatgtgcaaaagcgccggc

SEQ ID NO: 581 - DNA, 20 bp, Homo sapiens
  source 1..20: mol_type = genomic DNA
agagttgacggagacacagc

SEQ ID NO: 582 - DNA, 20 bp, Homo sapiens
  source 1..20: mol_type = genomic DNA
aagagttgacggagacacag

SEQ ID NO: 583 - DNA, 20 bp, Homo sapiens
  source 1..20: mol_type = genomic DNA
tctgttgcgtaaagagttga

SEQ ID NO: 584 - DNA, 20 bp, Homo sapiens
  source 1..20: mol_type = genomic DNA
ctctgttgcgtaaagagttg

SEQ ID NO: 585 - DNA, 20 bp, Homo sapiens
  source 1..20: mol_type = genomic DNA
cgcgcctggcttcctgcagt

SEQ ID NO: 586 - DNA, 20 bp, Homo sapiens
  source 1..20: mol_type = genomic DNA
atttcccaggttcgcactgc

SEQ ID NO: 587 - DNA, 20 bp, Homo sapiens
  source 1..20: mol_type = genomic DNA
gatttcccaggttcgcactg

SEQ ID NO: 588 - DNA, 20 bp, Homo sapiens
  source 1..20: mol_type = genomic DNA
tggcttcctgcagtgcgaac

SEQ ID NO: 589 - DNA, 20 bp, Homo sapiens
  source 1..20: mol_type = genomic DNA
ggcttcctgcagtgcgaacc

SEQ ID NO: 590 - DNA, 20 bp, Homo sapiens
  source 1..20: mol_type = genomic DNA
gcttcctgcagtgcgaacct

SEQ ID NO: 591 - DNA, 20 bp, Homo sapiens
  source 1..20: mol_type = genomic DNA
ttcggcaactgcgaagtcgc

SEQ ID NO: 592 - DNA, 20 bp, Homo sapiens
  source 1..20: mol_type = genomic DNA
tccaggcgacttcgcagttg

SEQ ID NO: 593 - DNA, 20 bp, Homo sapiens
  source 1..20: mol_type = genomic DNA
ttcagatcccttcggcaact

SEQ ID NO: 594 - DNA, 20 bp, Homo sapiens
  source 1..20: mol_type = genomic DNA
aggcgacttcgcagttgccg

SEQ ID NO: 595 - DNA, 20 bp, Homo sapiens
  source 1..20: mol_type = genomic DNA
ggcgacttcgcagttgccga

SEQ ID NO: 596 - DNA, 20 bp, Homo sapiens
  source 1..20: mol_type = genomic DNA
ttcgcagttgccgaagggat

SEQ ID NO: 597 - DNA, 20 bp, Homo sapiens
  source 1..20: mol_type = genomic DNA
gcagttgccgaagggatctg

SEQ ID NO: 598 - DNA, 20 bp, Homo sapiens
  source 1..20: mol_type = genomic DNA
tgccgaagggatctgaagaa

SEQ ID NO: 599 - DNA, 20 bp, Homo sapiens
  source 1..20: mol_type = genomic DNA
gccgaagggatctgaagaag

SEQ ID NO: 600 - DNA, 20 bp, Homo sapiens
  source 1..20: mol_type = genomic DNA
gggatgtgcaaaagcgccgg

SEQ ID NO: 601 - DNA, 20 bp, Homo sapiens
  source 1..20: mol_type = genomic DNA
ggatgtgcaaaagcgccggc

SEQ ID NO: 602 - DNA, 20 bp, Homo sapiens
  source 1..20: mol_type = genomic DNA
agagttgacggagacacagc

SEQ ID NO: 603 - DNA, 20 bp, Homo sapiens
  source 1..20: mol_type = genomic DNA
aagagttgacggagacacag

SEQ ID NO: 604 - DNA, 20 bp, Homo sapiens
  source 1..20: mol_type = genomic DNA
tctgttgcgtaaagagttga

SEQ ID NO: 605 - DNA, 20 bp, Homo sapiens
  source 1..20: mol_type = genomic DNA
ctctgttgcgtaaagagttg

SEQ ID NO: 606 - DNA, 20 bp, Homo sapiens
  source 1..20: mol_type = genomic DNA
cgcgcctggcttcctgcagt

SEQ ID NO: 607 - DNA, 20 bp, Homo sapiens
  source 1..20: mol_type = genomic DNA
atttcccaggttcgcactgc

SEQ ID NO: 608 - DNA, 20 bp, Homo sapiens
  source 1..20: mol_type = genomic DNA
gatttcccaggttcgcactg

SEQ ID NO: 609 - DNA, 20 bp, Homo sapiens
  source 1..20: mol_type = genomic DNA
tggcttcctgcagtgcgaac

SEQ ID NO: 610 - DNA, 20 bp, Homo sapiens
  source 1..20: mol_type = genomic DNA
ggcttcctgcagtgcgaacc

SEQ ID NO: 611 - DNA, 20 bp, Homo sapiens
  source 1..20: mol_type = genomic DNA
gcttcctgcagtgcgaacct

SEQ ID NO: 612 - DNA, 20 bp, Homo sapiens
  source 1..20: mol_type = genomic DNA
ttcggcaactgcgaagtcgc

SEQ ID NO: 613 - DNA, 20 bp, Homo sapiens
  source 1..20: mol_type = genomic DNA
tccaggcgacttcgcagttg

SEQ ID NO: 614 - DNA, 20 bp, Homo sapiens
  source 1..20: mol_type = genomic DNA
ttcagatcccttcggcaact

SEQ ID NO: 615 - DNA, 20 bp, Homo sapiens
  source 1..20: mol_type = genomic DNA
aggcgacttcgcagttgccg

SEQ ID NO: 616 - DNA, 20 bp, Homo sapiens
  source 1..20: mol_type = genomic DNA
ggcgacttcgcagttgccga

SEQ ID NO: 617 - DNA, 20 bp, Homo sapiens
  source 1..20: mol_type = genomic DNA
ttcgcagttgccgaagggat

SEQ ID NO: 618 - DNA, 20 bp, Homo sapiens
  source 1..20: mol_type = genomic DNA
gcagttgccgaagggatctg

SEQ ID NO: 619 - DNA, 20 bp, Homo sapiens
  source 1..20: mol_type = genomic DNA
tgccgaagggatctgaagaa

SEQ ID NO: 620 - DNA, 20 bp, Homo sapiens
  source 1..20: mol_type = genomic DNA
gccgaagggatctgaagaag

SEQ ID NO: 621 - DNA, 20 bp, Homo sapiens
  source 1..20: mol_type = genomic DNA
gggatgtgcaaaagcgccgg

SEQ ID NO: 622 - DNA, 20 bp, Homo sapiens
  source 1..20: mol_type = genomic DNA
ggatgtgcaaaagcgccggc

SEQ ID NO: 623 - DNA, 20 bp, Homo sapiens
  source 1..20: mol_type = genomic DNA
agagttgacggagacacagc

SEQ ID NO: 624 - DNA, 20 bp, Homo sapiens
  source 1..20: mol_type = genomic DNA
aagagttgacggagacacag

SEQ ID NO: 625 - DNA, 20 bp, Homo sapiens
  source 1..20: mol_type = genomic DNA
tctgttgcgtaaagagttga

SEQ ID NO: 626 - DNA, 20 bp, Homo sapiens
  source 1..20: mol_type = genomic DNA
ctctgttgcgtaaagagttg

SEQ ID NO: 627 - DNA, 20 bp, Homo sapiens
  source 1..20: mol_type = genomic DNA
cgcgcctggcttcctgcagt

SEQ ID NO: 628 - DNA, 20 bp, Homo sapiens
  source 1..20: mol_type = genomic DNA
atttcccaggttcgcactgc

SEQ ID NO: 629 - DNA, 20 bp, Homo sapiens
  source 1..20: mol_type = genomic DNA
gatttcccaggttcgcactg

SEQ ID NO: 630 - DNA, 20 bp, Homo sapiens
  source 1..20: mol_type = genomic DNA
tggcttcctgcagtgcgaac

SEQ ID NO: 631 - DNA, 20 bp, Homo sapiens
  source 1..20: mol_type = genomic DNA
ggcttcctgcagtgcgaacc

SEQ ID NO: 632 - DNA, 20 bp, Homo sapiens
  source 1..20: mol_type = genomic DNA
gcttcctgcagtgcgaacct

SEQ ID NO: 633 - DNA, 20 bp, Homo sapiens
  source 1..20: mol_type = genomic DNA
ttcggcaactgcgaagtcgc

SEQ ID NO: 634 - DNA, 20 bp, Homo sapiens
  source 1..20: mol_type = genomic DNA
tccaggcgacttcgcagttg

SEQ ID NO: 635 - DNA, 20 bp, Homo sapiens
  source 1..20: mol_type = genomic DNA
ttcagatcccttcggcaact

SEQ ID NO: 636 - DNA, 20 bp, Homo sapiens
  source 1..20: mol_type = genomic DNA
aggcgacttcgcagttgccg

SEQ ID NO: 637 - DNA, 20 bp, Homo sapiens
  source 1..20: mol_type = genomic DNA
ggcgacttcgcagttgccga

SEQ ID NO: 638 - DNA, 20 bp, Homo sapiens
  source 1..20: mol_type = genomic DNA
ttcgcagttgccgaagggat

SEQ ID NO: 639 - DNA, 20 bp, Homo sapiens
  source 1..20: mol_type = genomic DNA
gcagttgccgaagggatctg

SEQ ID NO: 640 - DNA, 20 bp, Homo sapiens
  source 1..20: mol_type = genomic DNA
tgccgaagggatctgaagaa

SEQ ID NO: 641 - DNA, 20 bp, Homo sapiens
  source 1..20: mol_type = genomic DNA
gccgaagggatctgaagaag

SEQ ID NO: 642 - DNA, 20 bp, Homo sapiens
  source 1..20: mol_type = genomic DNA
gggatgtgcaaaagcgccgg

SEQ ID NO: 643 - DNA, 20 bp, Homo sapiens
  source 1..20: mol_type = genomic DNA
ggatgtgcaaaagcgccggc

SEQ ID NO: 644 - DNA, 20 bp, Homo sapiens
  source 1..20: mol_type = genomic DNA
agagttgacggagacacagc

SEQ ID NO: 645 - DNA, 20 bp, Homo sapiens
  source 1..20: mol_type = genomic DNA
aagagttgacggagacacag

SEQ ID NO: 646 - DNA, 20 bp, Homo sapiens
  source 1..20: mol_type = genomic DNA
tctgttgcgtaaagagttga

SEQ ID NO: 647 - DNA, 20 bp, Homo sapiens
  source 1..20: mol_type = genomic DNA
ctctgttgcgtaaagagttg

SEQ ID NO: 648 - DNA, 20 bp, Homo sapiens
  source 1..20: mol_type = genomic DNA
cttcggcaactgcgaagtcg

SEQ ID NO: 649 - DNA, 20 bp, Homo sapiens
  source 1..20: mol_type = genomic DNA
aagagttgacggagacacag

SEQ ID NO: 650 - DNA, 20 bp, Homo sapiens
  source 1..20: mol_type = genomic DNA
cgcctccacgtgcgcggcgc

SEQ ID NO: 651 - DNA, 20 bp, Homo sapiens
  source 1..20: mol_type = genomic DNA
gtactctcctgcttcctcca

SEQ ID NO: 652 - DNA, 20 bp, Homo sapiens
  source 1..20: mol_type = genomic DNA
ccagaagtttccccatccca

SEQ ID NO: 653 - DNA, 20 bp, Homo sapiens
  source 1..20: mol_type = genomic DNA
tggactggttttattaattg

SEQ ID NO: 654 - DNA, 20 bp, Homo sapiens
  source 1..20: mol_type = genomic DNA
ttgagaggagtataggctga

SEQ ID NO: 655 - DNA, 20 bp, Homo sapiens
  source 1..20: mol_type = genomic DNA
ttcccagataggggagacag

SEQ ID NO: 656 - RNA, 25 nt, synthetic construct
  source 1..25: mol_type = other RNA
tgtaataggaacatctttgggatgg

SEQ ID NO: 657 - RNA, 25 nt, synthetic construct
  source 1..25: mol_type = other RNA
aaagataacaggtcaggaaccactg

SEQ ID NO: 658 - RNA, 25 nt, synthetic construct
  source 1..25: mol_type = other RNA
ataccagcaaaccagaataggcctc

SEQ ID NO: 659 - RNA, 25 nt, synthetic construct
  source 1..25: mol_type = other RNA
aataccagcaaaccagaataggcct

SEQ ID NO: 660 - RNA, 25 nt, synthetic construct
  source 1..25: mol_type = other RNA
ttaagagggattgtagccaactggg

SEQ ID NO: 661 - RNA, 25 nt, synthetic construct
  source 1..25: mol_type = other RNA
taaccagttccacaaagaagactgg

SEQ ID NO: 662 - RNA, 25 nt, synthetic construct
  source 1..25: mol_type = other RNA
aaatgaagacacacaagacctcttg

SEQ ID NO: 663 - RNA, 25 nt, synthetic construct
  source 1..25: mol_type = other RNA
agcaaatgaagacacacaagacctc

SEQ ID NO: 664 - RNA, 25 nt, synthetic construct
  source 1..25: mol_type = other RNA
atctaatctagcctggcctccactg

SEQ ID NO: 665 - RNA, 25 nt, synthetic construct
  source 1..25: mol_type = other RNA
tgtaggaactgccaaagtccttcgg

SEQ ID NO: 666 - RNA, 19 nt, synthetic construct
  source 1..19: mol_type = other RNA
tgaagacacacaagacctc

SEQ ID NO: 667 - RNA, 19 nt, synthetic construct
  source 1..19: mol_type = other RNA
caggagacaaggaagtgtg

SEQ ID NO: 668 - RNA, 19 nt, synthetic construct
  source 1..19: mol_type = other RNA
cactgaggataatagcctg

SEQ ID NO: 669 - RNA, 25 nt, synthetic construct
  source 1..25: mol_type = other RNA
caagaggtcttgtgtgtcttcattt

(19) United States
(12) Patent Application Publication
(10) Pub. No.: US 2026/0185089 A1
(43) Pub. Date: Jul. 2, 2026

(54) T CELL-NK CELL INTERACTION INHIBITORS FOR USE IN DISEASE TREATMENT

(71) Applicants: Deutsches Krebsforschungszentrum Stiftung des Öffentlichen Rechts, Heidelberg (DE); Universität Heidelberg, Heidelberg (DE)

(72) Inventors: Mirco Friedrich, Heidelberg (DE); Michael Kilian, Heidelberg (DE); Kevin Hai-Ning Lu, Heidelberg (DE); Michael Platten, Heidelberg (DE); Lukas Bunse, Heidelberg (DE)

(21) Appl. No.: 19/130,918

(22) PCT Filed: Oct. 31, 2023
(86) PCT No.: PCT/EP2023/080370

§ 371 Date: May 16, 2025

(30) Foreign Application Priority Data
Nov. 16, 2022 (EP) 22207782.8

(51) Int. Cl. C12N 15/113; A61K 39/00; A61P 35/00; C07K 16/28; C12Q 1/6886; G01N 33/50

(52) U.S. Cl. CPC C12N 15/113; A61P 35/00; C07K 16/2803; C07K 16/2827; C12Q 1/6886; G01N 33/5047; G01N 33/505; A61K 2039/505; C07K 2317/76; C12N 2310/20; C12Q 2600/158

(57) ABSTRACT

The present invention relates to a T cell-NK cell interaction inhibitor for use in improving in vivo persistence and/or activity of T cells. The present invention also relates to a method for producing a preparation of T cells from a cell population comprising T cells and known or suspected to comprise NK cells, to a method of identifying a subject amenable to treatment with a T cell-NK cell interaction inhibitor, to a method for identifying a T cell-NK cell interaction inhibitor, and to a use of a T cell-NK cell interaction inhibitor for expanding T cells.

The present invention relates to a T cell-NK cell interaction inhibitor for use in improving in vivo persistence and/or activity of T cells. The present invention also relates to a method for producing a preparation of T cells from a cell population comprising T cells and known or suspected to comprise NK cells, to a method of identifying a subject amenable to treatment with a T cell-NK cell interaction inhibitor, to a method for identifying a T cell-NK cell interaction inhibitor, and to a use of a T cell-NK cell interaction inhibitor for expanding T cells.

Natural killer (NK) cells constitute an integral part of antiviral and antitumor immune responses and as such strongly shape the host's immune repertoire (Cózar et al., Cancer Discovery (2021) doi: 10.1158/2159-8290.CD-20-0655). Beyond their antitumor functions, NK cells regulate immune responses in a multitude of disorders, including chronic viral infections, transplantation and autoimmunity (Gross al., in Immunology (2016) et Frontiers doi: 10.3389/fimmu.2016.00606). In murine tumor models, NK cells mediate early antitumor immune responses & Cerwenka, Immunobiology (2017) (Pahl doi: 10.1016/j.imbio.2015.07.012). In humans, the association of malignancies with primary NK cell immunodeficiency suggests an important role for NK cells in tumor immunosurveillance (Orange, Journal of Allergy and Clinical Immunology (2013) doi: 10.1016/j.jaci.2013.07.020).

Target cell engagement by NK cells is balanced via activating and inhibiting ligand-receptor interactions. Expression of activating ligands such as MHC class I chain-related protein A and B (MICA/B) and loss of inhibitory molecules can lead to direct killing of transformed tumor cells (Morvan & Lanier, Nature Reviews Cancer (2016) doi: 10.1038/nrc.2015.5). In general, NK cells are activated when they encounter infected, allogeneic or transformed cells resulting in rapid cytolytic activity as a consequence of a pre-primed state comparable to T cells (Bezman et al., Nat. Immunol. (2012) doi: 10.1038/ni.2395). NK cells can also eliminate activated T cells through Natural Killer Group 2D (NKG2D) receptor or lack of inhibitory NKG2A receptor ligands, thereby limiting the expansion of antiviral or autoreactive T cells (Waggoner, et al., Nature (2012) doi: 10.1038/nature10624; Lu et al., Immunity (2007) doi: 10.1016/j.immuni.2007.03.017).

Despite the above improvements, there is still a need for improved cancer treatments which provide advantageous options for the detection, treatment, and management of cancers. The technical problem underlying the present invention can be seen as the provision of means and methods for complying with the aforementioned needs. The technical problem is solved by the embodiments characterized in the claims and herein below.

In accordance, the present invention relates to a T cell-NK cell interaction inhibitor for use in improving in vivo persistence and/or activity of T cells.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof, thus, e.g. identifying “a cell” relates to identifying at least one cell, preferably to identifying a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods. The method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above.

As used herein, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (e.g. BLAST, GAP, BESTFIT, PASTA, or TFASTA), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. More preferably, the Basic Local Alignment Search Tool (BLAST) implementation is used with default parameter values for alignment. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, as used herein, e.g. an Fc or Fab fragment, but also e.g. a single-chain antibody, a bispecific antibody, and a nanobody may be referred to as fragments of an immunoglobulin.

Unless specifically indicated otherwise herein, the compounds specified, in particular the polynucleotides and polypeptides, may be comprised in larger structures, e.g. may be covalently or non-covalently linked to further sequences, carrier molecules, retardants, and/or excipients. In particular, polypeptides as specified may be comprised in fusion polypeptides comprising further (poly) peptides, which may serve e.g. as a tag for purification and/or detection, as a linker, or to extend the in vivo half-life of a compound. The term “detectable tag” refers to a stretch of amino acids which are added to or introduced into the fusion polypeptide; preferably, the tag is added C- or N-terminally to the fusion polypeptide. Said stretch of amino acids preferably allows for detection of the polypeptide by an antibody which specifically recognizes the tag; or it preferably allows for forming a functional conformation, such as a chelator; or it preferably allows for visualization, e.g. in the case of fluorescent tags. Preferred detectable tags are the Myc-tag, FLAG-tag, 6-His-tag, HA-tag, GST-tag or a fluorescent protein tag, e.g. a GFP-tag. These tags are all well known in the art. Other further (poly) peptides preferably comprised in a fusion polypeptide comprise further amino acids or other modifications which may serve as mediators of secretion, as mediators of blood-brain-barrier passage, as cell-penetrating peptides, and/or as immune stimulants. Further polypeptides or peptides to which the polypeptides may be fused are signal and/or transport sequences, e.g. an IL-2 signal sequence, and linker sequences.

The term “polypeptide”, as used herein, refers to a molecule consisting of several, typically at least 20 amino acids that are covalently linked to each other by peptide bonds. Molecules consisting of less than 20 amino acids covalently linked by peptide bonds are usually considered to be “peptides”. Preferably, the polypeptide comprises of from 50 to 1000, more preferably of from 75 to 1000, still more preferably of from 100 to 500, most preferably of from 110 to 400 amino acids. Preferably, the polypeptide is comprised in a fusion polypeptide and/or a polypeptide complex.

As used herein, the term “polypeptide variant” relates to any chemical molecule comprising at least one polypeptide as specified herein, having the indicated activity, but differing in structure from said specific polypeptide. Preferably, the polypeptide variant comprises a polypeptide having a contiguous amino acid sequence corresponding to at least 50%, preferably at least 75%, more preferably at least 85%, even more preferably at least 90%, most preferably at least 95%, of the amino acid sequence of the polypeptide specifically indicated. Moreover, it is to be understood that a polypeptide variant as referred to in accordance with the present invention shall have an amino acid sequence which differs due to at least one amino acid substitution, deletion and/or addition, wherein the amino acid sequence of the variant is still, preferably, at least 70%, more preferably at least 80%, even more preferably at least 90%, even more preferably at least 95%, still more preferably at least 98%, most preferably at least 99%, identical with the amino acid sequence of the specific polypeptide. The degree of identity between two amino acid sequences can be determined by algorithms well known in the art and as described herein above. Polypeptide variants referred to above may be allelic variants or any other species specific homologs, paralogs, or orthologs. Moreover, the polypeptide variants referred to herein include fragments of the specific polypeptides or the aforementioned types of polypeptide variants as long as these fragments and/or variants have the biological activity as specified. Such fragments may be or may be derived from, e.g., degradation products or splice variants of the polypeptides. Further included are variants which differ due to posttranslational modifications such as phosphorylation, glycosylation, ubiquitinylation, sumoylation, or myristylation, by including non-natural amino acids, and/or by being peptidomimetics.

The term “polynucleotide”, as used herein, refers to a linear or circular nucleic acid molecule. The polynucleotide of the present invention shall be provided, preferably, either as an isolated polynucleotide (i.e. isolated from its natural context) or in genetically modified form, preferably comprising at least one heterologous sequence. The term encompasses single—as well as double-stranded polynucleotides. Moreover, comprised are also chemically modified polynucleotides including naturally occurring modified polynucleotides such as glycosylated or methylated polynucleotides or artificially modified derivatives such as biotinylated polynucleotides, locked nucleic acids, and the like. The polynucleotides of the invention have the activity of encoding or being a T cell-NK cell interaction inhibitor. Methods for testing whether a given polynucleotide has the aforesaid activity are known in the art. E.g., preferably, a candidate polynucleotide of interest may be introduced into a host cell and the resulting amount and/or activity of being a T cell-NK cell interaction inhibitor is determined, preferably in comparison to a control. Unless specifically indicated otherwise, reference to specific polynucleotides herein preferably includes polynucleotide variants.

The term “polynucleotide variant”, as used herein, relates to a variant of a polynucleotide referred to herein comprising a nucleic acid sequence characterized in that the sequence can be derived from the aforementioned specific nucleic acid sequence by at least one nucleotide substitution, addition and/or deletion, wherein the polynucleotide variant shall have the activity as specified for the specific polynucleotide. Preferably, said polynucleotide variant is an ortholog, a paralog or another homolog of the specific polynucleotide. Also preferably, said polynucleotide variant is or is derived from a naturally occurring or a non-naturally occurring allele of the specific polynucleotide. Further, polynucleotide variants include polynucleotides comprising nucleic acid sequences which are at least 70%, preferably at least 80%, more preferably at least 90%, even more preferably at least 95%, still more preferably at least 98%, most preferably at least 99%, identical to the specifically indicated nucleic acid sequences. Moreover, also encompassed are polynucleotides which comprise nucleic acid sequences encoding amino acid sequences which are at least 70%, more preferably at least 80%, even more preferably at least 90%, even more preferably at least 95%, still more preferably at least 98, most preferably at least 99%, identical to the amino acid sequences specifically indicated. The percent identity values are, preferably, calculated over the entire amino acid or nucleic acid sequence region, preferably as specified herein above. The polynucleotides of the present invention either consist, essentially consist of, or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above.

The polynucleotide may be comprised in an expression construct. The term “expression construct”, as used herein, refers to a heterologous polynucleotide comprising the aforementioned polynucleotide as well as nucleic acid sequences required for expression of the polynucleotide. Typically, such additional nucleic acid sequences, which preferably are heterologous to the polynucleotide encoding a polypeptide as specified, may be promoter sequences, regulatory sequences and/or transcription termination sequences, such as terminators. Preferably, the expression construct is a eukaryotic expression construct, i.e. an expression construct comprising all elements required for expression, preferably inducible expression, in a eukaryotic host cell. Suitable expression control sequences are well known in the art and include in particular the CMV promoter or other constitutive promoters. However, inducible and/or cell-type specific promoters may be used as well.

The polynucleotide and/or the expression construct may be comprised in a vector. The term “vector”, as used herein, relates to any polynucleotide adapted for transferring, and preferably stably maintaining, the polynucleotide and/or the expression construct as specified herein above in a host cell. Thus, the term vector preferably encompasses phage, plasmid, and viral vectors as well artificial chromosomes, such as bacterial or yeast artificial chromosomes. Preferably, the vector is a plasmid or a virus-derived vector, preferably a replication-incompetent viral vector. Moreover, the term also relates to targeting constructs which allow for random or site-directed integration of the targeting construct into genomic DNA of a host cell. Such target constructs, preferably, comprise DNA of sufficient length for either homologous or heterologous recombination. The vector encompassing the polynucleotide and/or the expression construct as specified herein above, preferably, further comprises at least one selectable marker for propagation and/or selection of a host cell. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may e.g. be introduced by heat shock or electroporation techniques. Should the vector be a virus, it may be packaged in vitro using an appropriate packaging cell line or extract prior to application to host cells. Preferably, the vector is a vertebrate vector, more preferably a mammalian vector, or a shuttle vector. Preferably, the vector is an expression vector and/or a gene transfer or targeting vector. Methods which are well known to those skilled in the art can be used to construct recombinant polynucleotides and vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Preferably, the vector is an AAV vector, or a lentiviral vector.

In view of the above, the skilled person understands that references herein to a gene or its gene product include polynucleotide variants of the gene and its transcription product(s), i.e. in particular alleles, homologs, and mutants of the gene, as well as e.g. transcript variants, such as splice variants, and RNA editing variants of the RNA gene products. More preferably, reference to a gene relates to a gene and its naturally occurring alleles. Also, reference to a polypeptide gene product includes polypeptide variants as specified, in particular isoforms and/or muteins having the indicated degree of sequence identity and having the indicated biological activity. For those genes or gene products referred to by a Genbank Acc No or a similar designation, the respective sequence is herewith included to this description by reference.

The term “T cell” is understood by the skilled person to relate to a lymphocyte expressing at least one type of T cell receptor. Preferably, the T cell is a CD8+ T cell recognizing MHC class I molecules on the surface of target cells, or is a CD4+ T cell recognizing MHC class II molecules on the surface of target cells, more preferably is a CD8+ T cell. Preferably, the T cell is a cytotoxic T cell, more preferably a CD8+ cytotoxic T cell, which may also be referred to as “killer cell” or “killer T cells”. Also preferably, the T cell is a regulatory or helper T cell, more preferably a regulatory T cell. Preferably, the T cell is reactive to cancer cells, i.e. is a cancer-reactive T cell. Thus, preferably, the T cell expresses a TCR recognizing a cancer antigen, preferably a cancer-specific antigen. Preferably, the T cell is a B7H6+ T cell, more preferably is a B7H6+/CD4+ or a B7H6+/CD8+ T cell. Preferably, the T cell is a recombinant cell expressing a chimeric antigen receptor (CAR) and/or a recombinant T cell receptor.

Methods of producing appropriate recombinant T cells are known in the art. The T cell preferably is an activated T cell, preferably a T cell expressing, more preferably having upregulated, the receptor B7H6, wherein the term “upregulated” relates to a comparison to a non-activated T cell; method for obtaining appropriate activated and/or non-activated T cells are known in the art. Also preferably, the T cell is a tumor infiltrating T cell.

The terms “NK cell” and “natural killer cells” are known to the skilled person to relate to a group of innate lymphoid cells. Preferably, NK cells are identified or identifiable by the presence of the CD56 marker and the absence of the CD3 marker; thus, NK cells preferably are CD3⁺CD56⁺ NK cells. Preferably, the NK cell expresses at least one natural cytotoxicity triggering receptor (NCR) as specified herein below. As referred to herein, NK cells are not killer T cells, which are included as T cells as specified herein above; thus, NK cells as referred to herein are not T cells. Preferably, the NK cell interacts with at least one T cell, preferably via an interaction between at least one NCR with the T cell, preferably via an interaction between at least one NCR with B7H6 on a T cell. Preferably, the NK cell induces lysis of a T cell, preferably an activated T cell, more preferably via the aforesaid interaction. Thus, the NK cell preferably recognizes an activated T cells and induces lysis of said T cell. Preferably, said recognition is mediated by at least one NCR on the NK cell and at least one activation marker on the T cell, preferably B7H6. Also preferably, the NK cell is a tumor infiltrating NK cell.

The term “T cell-NK cell interaction”, as used herein, relates to an interaction between a T cell and an NK cell, both as specified herein above. Thus, the T cell-NK cell interaction preferably is an interaction between an activated T cell and an NK cell, which preferably is mediated by an interaction between at least one NCR on the NK cell with the T cell, preferably via a B7H6 on the T cell. Thus, the T cell-NK cell interaction preferably comprises and NCR-B7H6 interaction. Preferably, the T cell-NK cell interaction initiates lysis of the T cell.

The term “B7H6”, as used herein, relates to B7 homolog 6. Preferably, B7H6 is human B7H6 and B7H6 is encoded by the NCR3LG1 (natural killer cell cytotoxicity receptor 3 ligand 1) gene, which is known to be expressed in several isoforms. Thus, human B7H6 preferably has an amino acid sequence as shown in Genbank Acc. No. NP_001189368.1 or one of its isoforms. Preferably, B7H6 is encoded by the NCR3LG1 gene, encoding, in one of the isoforms expressed, an mRNA having a sequence as shown in Genbank Acc No. NM_001202439.3.

As used herein, the term “natural cytotoxicity triggering receptor”, abbreviated as “NCR”, relates to a group of transmembrane receptors expressed mainly on NK cells. NCRs are known to the skilled person under this designation. Preferably, the NCR is a human NCR, in particular in case the subject to be treated is a human subject. Preferably, the NCR is NCR1, more preferably comprising the amino acid sequence shown in Genbank Acc No. NP_001229286.1 or an isoform thereof; also preferably, the NCR is NCR2, more preferably comprising the amino acid sequence shown in Genbank Acc No. NP_001186438.1 or an isoform thereof, more preferably, the NCR is NCR3, more preferably comprising the amino acid sequence shown in Genbank Acc No.NP_001138938.1 or an isoform thereof. Most preferably, the NCR is NCR3, which is also known as “NKp30”.

The term “inhibitor” is known to the skilled person to relate to any compound causing the activity of a biological molecule or pathway to decrease, preferably significantly, compared to the activity in the absence of said inhibitor. Preferably, said inhibition is an inhibition by at least 20%, more preferably at least 50%, even more preferably at least 75%, even more preferably at least 90% of the value of an activity parameter in the absence of said inhibitor. The inhibition may, however, also be an inhibition by a factor of at least two, preferably at least five, more preferably at least ten. As is understood by the skilled person in view of the description herein below, inhibition may, however, also be complete abolishment of an activity present in the absence of said inhibitor. The effect of the inhibitor may be temporary, e.g. short-term over a time frame of hours or days, or long-lasting, e.g. over weeks or months, or may be permanent, in particular depending on the specific choice of the inhibitor. Preferably, said effect is temporary and lasts for of from 1 day to 6 months, preferably of from 2 days to 2 months, more preferably of from 3 days to 4 weeks, most preferably of from 1 to 4 weeks. As will also be understood by the skilled person, the effect may be local, i.e. topical at a site of administration, or may be systemic, e.g. after systemic administration of the inhibitor.

T cell-NK cell interaction inhibition preferably is put into effect by decreasing the amount and/or activity of an NCR on an NK cell, by decreasing the amount and/or activity of B7H6 on a T cell, and/or by at least partially inhibiting interaction of the NK cell and the T cell, preferably by inhibiting interaction between at least one NCR and B7H6. Preferably, the T cell-NK cell interaction inhibitor is a T cell-NK cell interaction decreasing compound, preferably a specific T cell-NK cell interaction decreasing compound. Preferably, the T cell-NK cell interaction inhibitor inhibits non-T cell-NK cell interaction-specific activity by at most 50%, preferably at most 25%, more preferably by at most 10% at a concentration inhibiting T cell-NK cell interaction by 90%. The activity of a T cell-NK cell interaction inhibitor is, preferably, determined in vitro by assaying the degree of T cell-NK cell interaction and/or by assaying activation of the B7H6 pathway in a T cell, as specified elsewhere herein, preferably as shown herein in the Examples. Also preferably, said T cell-NK cell interaction inhibitor prevents killing of anti-cancer T cells in vitro and/or in vivo. Thus activity of a T cell-NK cell interaction inhibitor may also be determined by determining survival of T cells in the presence of NK cells.

In accordance with the above, the term “T cell-NK cell interaction inhibitor” relates to each and every compound causing a reduction or abolishing T cell-NK cell interaction. Preferably, the T cell-NK cell interaction inhibitor is an inhibitor of NK cell-B7H6 interaction, preferably an inhibitor of NCR3-B7H6 interaction. Preferably, the T cell-NK cell interaction inhibitor is a transient T cell-NK cell interaction inhibitor, i.e. is a compound inducing T cell-NK cell interaction inhibition if and while contacted with T cells, as is e.g. the case if the T cell-NK cell interaction inhibitor is an antibody as specified herein below. More preferably, the T cell-NK cell interaction inhibitor is a permanent T cell-NK cell interaction inhibitor, i.e. is a compound inducing T cell-NK cell interaction inhibition which continues after the inhibitor is removed, as is e.g. the case if the T cell-NK cell interaction inhibitor is a CRISPR/Cas oligonucleotide as specified herein below. As referred to herein, a T cell-NK cell interaction inhibitor inhibiting T cell-NK cell interaction in vitro for at least ten doublings of a T cell population after its removal is deemed a permanent T cell-NK cell interaction inhibitor.

Preferably, the T cell-NK cell interaction inhibitor is a direct T cell-NK cell interaction inhibitor, i.e. a compound binding to, preferably specifically binding to, at least one NCR and/or to B7H6, and thereby inhibiting T cell-NK cell interaction. More preferably, the direct T cell-NK cell interaction inhibitor is an inhibitor polypeptide, an inhibitor polynucleotide, or a non-polypeptide non-polynucleotide inhibitor macromolecule. Preferably, the direct T cell-NK cell interaction inhibitor is a compound binding to at least one epitope in at least one NCR and/or in B7H6, preferably an epitope including at least one amino acid of an interaction epitope and/or an active center of at least one NCR and/or of B7H6. The skilled person is aware of methods suitable for determining binding of a direct inhibitor to at least one NCR and/or to B7H6, e.g. staining NCR- or B7H6-positive cells or of extracts from such cells with a candidate direct inhibitor, wherein said inhibitor is coupled to a detectable label, preferably a colored and/or fluorescent dye; ELISA methods; surface plasmon resonance methods, and the like.

The term “inhibitor polypeptide” is used herein to relate to any and all polypeptides or peptides binding to at least one NCR and/or to B7H6 and inhibiting interaction as specified herein above. Thus, the inhibitor polypeptide preferably binds to the extracellular domain(s) of at least one NCR and/or B7H6. Preferably, the inhibitor polypeptide is an antagonist, more preferably a competitive antagonist or a non-competitive antagonist. Preferably, the inhibitor polypeptide is selected from the list consisting of an antibody, an aptamer, an anticalin, and a Designed Ankyrin Repeat Protein (DARPin).

As used herein, the term “antibody” relates to a soluble immunoglobulin from any of the classes IgA, IgD, IgE, IgG, or IgM, or fragments thereof, having the activity of directly interacting with at least one NCR and/or to B7H6 and inhibiting interaction as specified herein above. Antibodies against at least one NCR and/or B7H6 or fragments thereof are commercially available and can be prepared by well-known methods using a purified NCR or B7H6 polypeptide or a suitable fragment derived therefrom as an antigen. A fragment which is suitable as an antigen may be identified by antigenicity determining algorithms well known in the art. Suitable fragments may also be obtained from an NCR polypeptide and/or B7H6 polypeptide by proteolytic digestion, may be synthetic peptides, or may be recombinantly expressed. Suitability of an antibody thus generated as a T cell-NK cell interaction inhibitor can be tested by an assay as described elsewhere herein. Preferably, the antibody of the present invention is a monoclonal antibody, a human, primatized, chimerized, or humanized antibody, or a fragment thereof. More preferably, the antibody is a single chain antibody, a single-domain antibody, a nanobody, or an antibody fragment, such as Fab, scFab, and the like. Also comprised as antibodies of the present invention are a bispecific antibody, a synthetic antibody, or a chemically modified derivative of any of the aforesaid antibodies. Preferably, the antibody of the present invention shall specifically bind (i.e. does not cross react with other polypeptides or peptides) to an NCR polypeptide and/or B7H6 polypeptide as specified above. Specific binding can be tested by various well-known techniques. Antibodies or fragments thereof can be obtained by using methods described, e.g., in Harlow and Lane “Antibodies, A Laboratory Manual”, CSH Press, Cold Spring Harbor, 1988. Monoclonal antibodies can be prepared by the techniques originally described in Köhler and Milstein, Nature. 1975. 256:495; and Galfré, Meth. Enzymol. 1981, 73:3, which comprise the fusion of mouse myeloma cells to spleen cells derived from immunized mammals. Preferably, the antibody is an antibody as specified above or a polypeptide derivative thereof; more preferably, the antibody is an antibody as specified above. Preferably, the antibody is reactive against recombinant human NCR3, preferably sterically blocking the interaction between NCR3 and B7H6, or neutralizing NCR3 binding domains and/or is a B7H6 blocking antibody. Also preferably, the antibody is a monoclonal antibody reactive against recombinant human NCR3, sterically blocking the interaction between NCR3 and B7H6; or neutralizing NCR3 binding domains and/or is a monoclonal B7H6 blocking antibody.

As used herein, the term “aptamer” relates to a polynucleotide or polypeptide binding specifically to a target molecule by virtue of its three-dimensional structure. Preferably, the aptamer is a peptide aptamer, a “peptide aptamer” preferably being a peptide specifically interacting with at least one NCR and/or B7H6 and, thereby, inhibiting T cell-NK cell interaction, preferably NCR-B7H6 interaction, as specified herein above. Peptide aptamers, preferably, are peptides comprising 8-80 amino acids, more preferably 10-50 amino acids, and most preferably 15-30 amino acids. They can e.g. be isolated from randomized peptide expression libraries in a suitable host system like baker's yeast (see, for example, Klevenz et al., Cell Mol Life Sci. 2002, 59:1993-1998). A peptide aptamer, preferably, is a free peptide; it is, however, also contemplated that a peptide aptamer is fused to a polypeptide serving as “scaffold”, meaning that the covalent linking to said polypeptide serves to fix the three-dimensional structure of said peptide aptamer to a specific conformation. Preferably, the aptamer is an aptamer as specified above or a polypeptide or polynucleotide derivative thereof; more preferably, the aptamer is an aptamer as specified above.

As used herein, the term “anticalin” relates to an artificial polypeptide derived from a lipocalin specifically binding at least one NCR and/or B7H6 and thereby inhibiting T cell-NK cell interaction, preferably NCR-B7H6 interaction, as specified herein above. Similarly, a “Designed Ankyrin Repeat Protein” or “DARPin”, as used herein, is an artificial polypeptide, comprising several ankyrin repeat motifs, specifically binding at least one NCR and/or B7H6 and thereby inhibiting T cell-NK cell interaction, preferably NCR-B7H6 interaction. Preferably, the anticalin or DARPin is an anticalin or DARPin as specified above or a polypeptide derivative thereof; more preferably, the anticalin or DARPin is an anticalin or DARPin as specified above.

The term “non-polypeptide non-polynucleotide inhibitor macromolecule”, as used herein, relates to any and all molecules having the property of being a direct inhibitor of T cell-NK cell interaction and being a macromolecule, preferably a biological macromolecule, the term “macromolecule”, as used herein, relating to molecules having a molecular mass of more than 1 kDa, preferably more than 1.5 kDa, more preferably more than 2 kDa, most preferably more than 2.5 kDa. As is understood by the skilled person, the non-polypeptide non-polynucleotide inhibitor macromolecule preferably is not a polypeptide and is not a polynucleotide, although the non-polypeptide non-polynucleotide inhibitor macromolecule may comprise substructures belonging to one of the aforesaid molecule classes. Preferably the non-polypeptide non-polynucleotide inhibitor macromolecule is a polysaccharide and/or a lipid.

Also preferably, the T cell-NK cell interaction inhibitor is a compound not directly inhibiting T cell-NK cell interaction, but still reducing, preferably significantly, T cell-NK cell interaction in a target cell, i.e. is an indirect inhibitor. Preferably, the indirect T cell-NK cell interaction inhibitor is a compound decreasing the amount of at least one NCR and/or B7H6 in a target cell. Preferably, the indirect T cell-NK cell interaction inhibitor specifically binds to a polynucleotide encoding at least one NCR and/or B7H6, preferably thereby significantly reducing, more preferably preventing, expression of said at least one NCR and/or B7H6. Also preferably, the indirect T cell-NK cell interaction inhibitor is or binds to, preferably specifically binds to, a transcriptional regulator of at least one gene encoding an NCR and/or a B7H6, preferably thereby significantly reducing, more preferably preventing, transcription of said gene. Thus, the indirect T cell-NK cell interaction inhibitor may be a transcriptional repressor of NCR and/or B7H6 transcription, or may be an inhibitor of a transcriptional activator of NCR and/or B7H6 transcription. The indirect T cell-NK cell interaction inhibitor may, however, also be a compound accelerating degradation of at least one NCR and/or B7H6 in a cell or a compound decreasing the concentration of at least one NCR and/or B7H6 activator.

Preferably, the indirect T cell-NK cell interaction inhibitor is a polynucleotide, more preferably a polynucleotide inhibiting expression or inducing degradation of an NCR mRNA and/or a B7H6 mRNA. More preferably, the indirect T cell-NK cell interaction inhibitor is selected from the group consisting of an shRNA, an siRNA, an miRNA agent, a ribozyme, an antisense molecule/an inhibitory oligonucleotide, and a CRISPR/Cas oligonucleotide. It is understood by the skilled person that inhibition of expression or induction of degradation of a specific RNA can be achieved in various ways. It is also understood by the skilled person that the exact embodiment of a polynucleotide being an indirect T cell-NK cell interaction inhibitor of the present invention will depend on the treatment intended.

Preferably, the indirect T cell-NK cell interaction inhibitor is a ribozyme. The term “ribozyme” as used herein, refers to catalytic RNA molecules possessing a well-defined tertiary structure that allows for catalyzing either the hydrolysis of one of their own phosphodiester bonds (self-cleaving ribozymes), or the hydrolysis of bonds in other RNAs, but they have also been found to catalyze the aminotransferase activity of the ribosome. The ribozymes envisaged in accordance with the present invention are, preferably, those which specifically hydrolyze their target RNAs, preferably an NCR mRNA and/or a B7H6 mRNA. In particular, hammerhead ribozymes are preferred in accordance with the present invention. How to generate and use such ribozymes is well known in the art (see, e.g., Hean & Weinberg (2008), RNA and the Regulation of Gene Expression: A Hidden Layer of Complexity, Chapter 1. Caister Academic Press).

More preferably, the indirect T cell-NK cell interaction inhibitor is an antisense oligonucleotide. The term “antisense oligonucleotide” is known to the skilled person and relates to an oligonucleotide hybridizing to a target RNA, causing the formation of a DNA/RNA hybrid. Said DNA/RNA hybrid is a substrate for RNase H, which degrades the RNA portion of said DNA/RNA hybrid. Thus, the antisense oligonucleotide comprises at least five, preferably at least seven, more preferably at least nine, most preferably at least ten, DNA nucleotides.

Preferably, the antisense oligonucleotide has a length of at least 15 nucleotides, preferably at least 18 nucleotides, still more preferably at least 20 nucleotides, preferably complementary to an NCR mRNA and/or a B7H6 mRNA sequence as specified herein above.

Also more preferably, the indirect T cell-NK cell interaction inhibitor is a polynucleotide inducing RNA interference. As used herein, “RNA interference (RNAi)” refers to sequence-specific, post-transcriptional gene silencing of a selected target gene by degradation of RNA transcribed from the target gene (target RNA). Target RNAs, preferably, are mRNAs, i.e. RNAs transcribed from a gene encoding an NCR and/or B7H6 as specified herein above. It is to be understood that silencing as used herein does not necessarily mean the complete abolishment of expression. RNAi, preferably, reduces expression by at least 40%, more preferably at least 60%, even more preferably at least 80%, most preferably at least 90% as compared to the expression level in a reference without RNAi. RNAi requires in the target cell the presence of dsRNAs that are homologous in sequence to the target RNAs. The term “dsRNA” refers to RNA having a duplex structure comprising two complementary and anti-parallel nucleic acid strands. The RNA strands forming the dsRNA may have the same or a different number of nucleotides, whereby one of the strands of the dsRNA can be the target RNA. It is, however, also contemplated by the present invention that the dsRNA is formed between two sequence stretches on the same RNA molecule, e.g. by formation of a stem-loop structure. RNAi may be used to specifically inhibit expression of the target RNAs of the present invention in vivo. Accordingly, it may be used for the medical uses as specified elsewhere herein. For such therapeutic approaches, expression constructs for siRNA may be introduced into target cells of the host. Accordingly, siRNA may be combined efficiently with other therapy approaches. Methods relating to the use of RNAi to silence genes in animals, including mammals, are known in the art.

Thus, the indirect T cell-NK cell interaction inhibitor, preferably is an RNAi agent. As used herein, the term “RNAi agent” refers to an shRNA, a siRNA agent, or a miRNA agent as specified below. The RNAi agent of the present invention is of sufficient length and complementarity to stably interact with the target RNA, i.e. it comprises at least 15, at least 17, at least 19, at least 21, at least 22 nucleotides complementary to the target RNA. By “stably interact” is meant interaction of the RNAi agent or its products produced by the target cell with a target RNA, e.g., by forming hydrogen bonds with complementary nucleotides in the target RNA under physiological conditions.

The term “siRNA agent” as meant herein encompasses: a) a dsRNA consisting of at least 15, at least 17, at least 19, at least 21 consecutive nucleotides base-paired, i.e. forming hydrogen bonds with complementary nucleotides. b) a small interfering RNA (siRNA) molecule or a molecule comprising an siRNA molecule. The siRNA is a single-stranded RNA molecule with a length, preferably, greater than or equal to 15 nucleotides and, preferably, a length of 15 to 49 nucleotides, more preferably 17 to 30 nucleotides, and most preferably 17 to 30 nucleotides, preferably 17, 18, 19, 20, 21, 22, 23, 24, 25, 26, 27, 28, 29, or 30 nucleotides. According to the present invention, the term “molecule comprising an siRNA molecule” includes RNA molecules from which an siRNA is processed by a cell, preferably by a mammalian cell. Thus, a molecule comprising an siRNA molecule, preferably, is a small hairpin RNA, also known as shRNA. As used herein, the term “shRNA” relates to a, preferably artificial, RNA molecule forming a stem-loop structure comprising at least 10, preferably at least 15, more preferably at least 17, most preferably at least 20 nucleotides base-paired to a complementary sequence on the same mRNA molecule (“stem”), i.e. as a dsRNA, separated by a stretch of non-base-paired nucleotides (“loop”). c) a polynucleotide encoding a) or b), wherein, preferably, said polynucleotide is operatively linked to an expression control sequence. Thus, the function of the siRNA agent to inhibit expression of the target gene can be modulated by said expression control sequence. Preferred expression control sequences are those, which can be regulated by exogenous stimuli, e.g. the tet operator, whose activity can be regulated by tetracycline, or heat inducible promoters. Alternatively or in addition, one or more expression control sequences can be used which allow tissue-specific expression of the siRNA agent. In view of the above, the siRNA preferably binds to a sequence comprised in an mRNA transcribed from an NCR3LG1 gene, e.g. as shown in Genbank Acc No. XM_047426906.1, or a transcript variant thereof. Thus the siRNA preferably comprises a targeting sequence, i.e. a sequence specifically hybridizing to the NCR3LG1 mRNA, comprising a nucleic acid sequence selected from SEQ ID NOs: 656 to 669. Preferably, the siRNA comprises a ribonucleotide sequence selected from SEQ ID NOs: 656 to 669. More preferably, the siRNA comprises a ribonucleotide sequence corresponding to any one of SEQ ID NO:656 to 669 and a ribonucleotide sequence corresponding to the reverse complement of said sequence, preferably connected by a loop sequence.

Also more preferably, the RNAi agent is an miRNA agent. An “miRNA agent” as meant herein encompasses: a) a pre-microRNA, i.e. an mRNA comprising at least 30, at least 40, at least 50, at least 60, at least 70 nucleotides base-paired to a complementary sequence on the same mRNA molecule (“stem”), i.e. as a dsRNA, separated by a stretch of non-base-paired nucleotides (“loop”). b) a pre-microRNA, i.e. a dsRNA molecule comprising a stretch of at least 19, at least 20, at least 21, at least 22, at least 23, at least 24, at least 25 base-paired nucleotides formed by nucleotides of the same RNA molecule (stem), separated by a loop. c) a microRNA (miRNA), i.e. a dsRNA comprising at least 15, at least 17, at least 18, at least 19, at least 21 nucleotides on two separate RNA strands. d) a polynucleotide encoding a) or b), wherein, preferably, said polynucleotide is operatively linked to an expression control sequence as specified above.

Most preferably, the indirect T cell-NK cell interaction inhibitor comprises at least one, preferably two, CRISPR/Cas oligonucleotides. The CRISPR/Cas system has been known for several years as a convenient system for inducing knock-out mutations, i.e. deletions, preferably of chromosomal genes. The skilled person knows how to design appropriate oligonucleotides, which are, preferably, expressed from a vector, to induce deletion of a DNA sequence of interest. Preferably, said deletion is a partial deletion, more preferably deletion of a portion of the gene essential for function; most preferably said deletion is a complete deletion of at least the whole coding region. As is known in the art, single CRISPR/Cas oligonucleotides can be used to introduce short insertions, deletions, and/or frameshifts in a coding sequence of interest, while two CRISPR/Cas oligonucleotides may be used to mediate larger deletions or deletions of exons, coding regions and/or whole genes. As referred to herein, two CRISPR/Cas oligonucleotides together mediating such partial or complete deletion of at least one exon, coding region and/or whole gene are referred to as a “pair of CRISPR/Cas oligonucleotides”. Preferably, the CRISPR/Cas oligonucleotide comprises at least one targeting sequence, the term “targeting sequence”; as referred to herein, relating to a subsequence of a CRISPR/Cas oligonucleotide mediating recognition, i.e. preferably specifically hybridizing to, a target sequence in a gene which is to be inactivated. Preferably, the CRISPR/Cas oligonucleotide is a Cas9 or Cas12 compatible oligonucleotide. As the skilled person is aware of, different Cas enzymes may have different requirements regarding targeting sequence, guide spacer length, total guide length, length and type of PAM sequence, and the like. These requirements are known in the art and the skilled person can select an appropriate CRISPR/Cas oligonucleotide configuration for the specific Cas nuclease used. Preferably, the targeting sequence comprises a nucleic acid sequence as shown in any one of SEQ ID NO:1 to 655, preferably of SEQ ID NO: 1 to 10. Preferably, the Cas nuclease is a Streptococcus pyogenes Cas nuclease and the CRISPR/Cas oligonucleotide comprises a targeting sequence comprising a nucleic acid sequence as shown in any one of SEQ ID NOs: 1 to 332, or the Cas nuclease is a Staphylococcus aureus Cas nuclease and the CRISPR/Cas oligonucleotide comprises a targeting sequence comprising a nucleic acid sequence as shown in any one of SEQ ID NOs: 333 to 647; or the Cas nuclease is a Neisseria meningitidis Cas nuclease and the CRISPR/Cas oligonucleotide comprises a targeting sequence comprising a nucleic acid sequence as shown in any one of SEQ ID NOs: 648 to 650. More preferably, the Cas nuclease is a Streptococcus pyogenes Cas nuclease and the CRISPR/Cas oligonucleotide comprises a targeting sequence comprising a nucleic acid sequence as shown in any one of SEQ ID NOs: 1 to 332, preferably 1 to 10.

Also preferably, the indirect T cell-NK cell interaction inhibitor is a polypeptide comprising a lysosome-degradation sequence, preferably a chaperone-mediated autophagy-targeting motif (CTM). Preferably, said CTM-comprising polypeptide specifically binds to an NCR and/or B7H6; e.g. the CTM-comprising polypeptide may further comprise an antibody specifically binding to an NCR and/or B7H6. As the skilled person will understand, the CTM-conjugated antibody does not necessarily have to be an inhibitory antibody as specified herein above; it is, however, preferred that the antibody is an antibody specific for at least one NCR and/or B7H6. Preferably, the CTM-comprising polypeptide specifically binding to an NCR and/or B7H6 binds to said NCR and/or B7H6 with a K_{D} value at least 10fold, preferably at least 20fold, more preferably at least 50fold, most preferably at least 100fold higher than the K_{D} value of said CTM-comprising polypeptide for other surface polypeptides of an NK cell or a T cell. More preferably, the CTM-comprising polypeptide specifically binding to an NCR and/or B7H6 does not detectably bind to other cellular proteins. As will be understood by the skilled person, in case the indirect T cell-NK cell interaction inhibitor is a CTM-comprising polypeptide, said CTM-comprising polypeptide does not have to be, but may be, a direct T cell-NK cell interaction inhibitor. Thus, preferably, the CTM-comprising polypeptide also is a direct T cell-NK cell interaction inhibitor.

The term “persistence” is understood by the skilled person to relate to the continued existence and/or activity of an entity; thus, persistence of cells, in particular T cells, as referred to herein, relates to survival and, preferably, continued activity, of said cells, in particular in the presence of NK cells. As the skilled person understands, NK cells will typically be present in in vivo settings, so in vivo persistence of T cells preferably relates to survival and more preferably continued activity of T cells in an in vivo setting, i.e. in the body of a subject. Preferably, said subject comprises NK cells at normal levels, i.e. at levels corresponding to those found on average in a cohort of apparently healthy subjects.

In accordance with the above, the term “improving in vivo persistence of T cells” relates to increasing, preferably in a statistically significant manner, probability of T cells to survive in vivo settings. Thus, improving persistence may comprise increasing in vivo the number of living T cells at a given point in time, increasing the average time span of survival of T cells, or improving any other parameter indicative of T cell survival, all preferably compared to the corresponding parameter(s) determined in a control without T cell-NK cell interaction inhibitor. Said improved persistence may be specific for a subset of T cells, e.g. only in CD4+ T cells, only in tumor-antigen specific T cells, or the like. In a preferred embodiment, the improved persistence is improved persistence of CD8+ T cells. More preferably, T cell persistence is improved for all activated T cells, still more preferably all T cells. Preferably, at least 5%, more preferably at least 10%, even more preferably at least 25% of said T cells are activated T cells as specified herein above.

Similarly, “improving in vivo activity of T cells” relates to increasing activity of T cells in vivo settings. Said increase may be specific for a subset of T cells, e.g. only in CD4+ T cells, only in tumor-antigen specific T cells, or the like. In a preferred embodiment, the improved activity is improved activity of CD8+ T cells. Improving in vivo activity of T cells may in particular comprise increasing in vivo the number of activated T cells at a given point in time, increasing the average time span of a T cell being in an activated state, increasing the number of cells lysed by said T cell, i.e. increasing cytolytic activity, or improving any other parameter indicative of T cell activity, all preferably compared to the corresponding parameter(s) determined in a control without T cell-NK cell interaction inhibitor. In a preferred embodiment, improving in vivo activity of T cells is causing activated T cells, in particular activated genetically modified T cells, to be present at a fraction of at least 10%, preferably at least 20%, more preferably of at least 30% of all T cells, in particular of all genetically modified T cells.

Improving in vivo persistence and/or activity of T cells may be particularly advantageous in diseases which benefit from an increased activity of T cells, i.e. in particular cancer, infectious diseases like viral infections, and the like; or in diseases where disproportional loss of activated T cells underlies the disease, in particular HIV infection, severe combined immunodeficiency (SCID), Wiskott-Aldrich syndrome, ataxia telangiectasia, DiGeorge syndrome (22q11. 2 deletion syndrome), immuno-osseous dysplasias, dyskeratosis congenita, and chronic mucocutaneous candidiasis. Thus, improving in vivo persistence and/or activity of T cells preferably is treatment and/or prevention of cancer and/or of infectious disease. Preferably, said treating and/or preventing comprises administration of in vitro expanded T cells. More preferably, treating and/or preventing comprises administering tumor infiltrating lymphocytes (TILs) and/or T cells expressing a chimeric antigen receptor (CAR) and/or T cells expressing a recombinant T cell receptor (rTCR), which preferably were generated and/or expanded in vitro.

Preferably, the T cell-NK cell interaction inhibitor is administered systemically, preferably orally or parenterally, e.g. by intravenous administration, or is administered topically, preferably intra-tumorally, topically on a body surface, or by inhalation; in case of cancer treatment, topical administration may be intratumoral or peritumoral, and/or topical at a site of tumor excision. Administration may, however, also be into a blood vessel, typically an artery, afferent to an intended site of effect, such as a tumor. However, depending on the nature of the formulation and the desired therapeutic application, the medicament may be administered by other routes as well.

The term “cancer”, as used herein, relates to a disease of an animal, including man, characterized by uncontrolled growth by a group of body cells (“cancer cells”). This uncontrolled growth may be accompanied by intrusion into and destruction of surrounding tissue (infiltration) and possibly spread of cancer cells to other locations in the body (metastasis). Preferably, also included by the term cancer is a recurrence of a cancer (relapse). Thus, preferably, the cancer is a solid cancer, a metastasis, or a relapse thereof. Also preferably, the cancer is a non-solid cancer, in particular a leukemia, in particular a relapse or an advanced stage leukemia. Preferably, the cancer is selected from the list consisting of acute myeloid leukemia (AML), acute lymphoblastic leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, fibrosarcoma, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sézary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenström macroglobulinemia, and wilms tumor. More preferably, the cancer is a glioblastoma, a multiple myeloma, or is colorectal cancer.

The terms “treating” and “treatment” refer to an amelioration of a disease or disorder referred to herein or the symptoms accompanied therewith to a significant extent; as used herein, the term includes prevention of deterioration of a disease, disorder, or symptoms associated therewith. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50% at least 60%, at least 70%, at least 80%, or at least 90% of the subjects of a given cohort or population. Preferably, treating comprises inhibiting proliferation, more preferably killing, of cancer cells. Preferably, treating cancer is reducing tumor and/or cancer cell burden in a subject. As will be understood by the skilled person, effectiveness of treatment of e.g. cancer is dependent on a variety of factors including, e.g. cancer stage and cancer type. Also preferably, cancer treatment further comprises at least one of chemotherapy, immunotherapy, surgery, and radiotherapy.

The terms “preventing” and “prevention” refer to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification. In the context of cancer treatment, preventing in particular relates to preventing cancer development, preventing metastasis formation, and/or preventing relapse, preferably relates to preventing metastasis formation and/or preventing relapse.

The term “infectious disease” is, in principle, understood by the skilled person. Preferably, the term as used herein relates to an infectious disease in which the replicative cycle of the infectious agent causing the disease comprises at least one stage in which a gene product of the infectious agent is present in an infected cell and, preferably, at least one peptide which is a gene product of the infectious agent is presented via MHC class I and/or class II by cells of the infected subject. Thus the infectious disease, preferably, is a viral infection, preferably is immunodeficiency virus infection, herpes virus infection, papillomavirus infection, hepatitis B virus infection, or a SARS-COV infection, preferably a SARS-COV-2 infection.

The T cell-NK cell interaction inhibitor may be comprised in a pharmaceutical composition. Thus, the present invention also relates to a pharmaceutical composition comprising the T cell-NK cell interaction inhibitor of the present invention for use in treating and/or preventing cancer.

The terms “medicament” and “pharmaceutical composition” are used essentially interchangeably herein and are, in principle, known to the skilled person. As referred to herein, the terms relate to any composition of matter comprising the specified active agent(s) as pharmaceutically active compound(s) and one or more excipient. The pharmaceutically active compound(s) can be present in liquid or dry, e.g. lyophilized, form. It will be appreciated that the form and character of the pharmaceutical acceptable excipient, e.g. carrier or diluent, is dictated by the amount of active ingredient with which it is to be combined, the route of administration, and other well-known variables. The excipient(s) must be acceptable in the sense of being compatible with the other ingredients of the formulation and being not deleterious to the recipient thereof. The excipient employed may include a solid, a gel, or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, physiological saline, Ringer's solutions, dextrose solution, and Hank's solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pennsylvania. The excipient(s) is/are selected so as not to affect the biological activity of the combination. The excipient may, however, also be selected to improve uptake of the active agent into a host cell, in particular a target cell. Thus, the excipient may also be a viral particle and/or a lipid vesicle, preferably a viral particle and/or a lipid vesicle known to mediate entry and/or fuse with the target cell of interest.

The medicament is, preferably, administered by a route as specified herein above. A therapeutically effective dose refers to an amount of the T cell-NK cell interaction inhibitor to be used which prevents, ameliorates or cures the symptoms accompanying a disease or condition referred to in this specification. Therapeutic efficacy and toxicity of a drug can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and by clinical factors. As is well known in the medical arts, dosages for any one patient may depend upon many factors, including the patient's size, age, the particular formulation of the medicament to be administered, sex, time and route of administration, general health, and other drugs being administered concurrently. The medicament referred to herein is, preferably, administered at least once, e.g. as a bolus. However, the medicament may be administered more than one time and, preferably, at least twice, e.g. permanently or periodically after defined time windows. Progress can be monitored by periodic assessment. Dosage recommendations may be indicated in the prescriber or user instructions in order to anticipate dose adjustments depending on the considered recipient.

The medicament according to the present invention may comprise further active agents in addition to the aforementioned active agent(s). Preferably, the pharmaceutically active compound according to the invention is to be applied together with at least one further drug and, thus, may be formulated together with this at least one further drug as a medicament. More preferably, in case of cancer treatment, said at least one further active agent is a chemotherapeutic agent or a further immunotherapeutic agent, such as an immune checkpoint modulator. Also, it is to be understood that the formulation of a pharmaceutical composition preferably takes place under GMP standardized conditions or the like in order to ensure quality, pharmaceutical safety, and effectiveness of the medicament.

The term “subject”, as referred to herein, relates to a vertebrate animal, preferably a mammal, in particular a livestock, companion, or laboratory animal. Most preferably, subject is a human. Preferably, the subject has been diagnosed to suffer from cancer as specified herein above. Also preferably, the subject has been identified to comprise anti-tumor T cells and/or is planned to be administered T cells.

Advantageously, it was found in the work underlying the present invention that NK cells mediate killing of T cells via B7H6, which may limit antitumor immunity by suppression of tumor-reactive T cells. Also, it was found that NK cell-mediated cytolysis of B7H6 expressing activated T cells is relevant in limiting the persistence of chimeric antigen receptor (CAR) T cells in vivo and that depletion of NK cells or CRISPR-Cas-mediated deletion of B7H6 on T cells amplifies CAR T cell expansion.

The definitions made above apply mutatis mutandis to the following. Additional definitions and explanations made further below also apply for all embodiments described in this specification mutatis mutandis.

The present invention further relates to a method for producing a preparation of T cells from a cell population comprising T cells and known or suspected to comprise NK cells, said method comprising (i) contacting said cell population with a T cell-NK cell interaction inhibitor, (ii) expanding T cells, and (iii) thereby, producing a preparation of T cells.

The method for producing a preparation of T cells preferably is an in vitro method; the method may, however, also be performed in vivo, e.g. as a part of a method of treating and/or preventing disease as specified herein below. Also, the method may comprise further steps, e.g. providing a cell population comprising T cells, e.g. a buffy coat or a PBMC preparation, for step (i), contacting the cell population with additional chemical agents, such as chemokines, nutrients, and the like, in step (i) and/or (ii), and optionally retransferring the preparation of T cells of step (iii) into a subject. Also, one or more steps may be assisted or performed by automated equipment.

As used herein, the term “cell population comprising T cells” relates to any composition comprising at least T cells. Said composition may be a sample withdrawn from a subject comprising T cells, e.g. a blood sample, may be an enriched preparation of T cell, e.g. in a growth medium, may be a cell population maintained in vitro, or any composition deemed suitable by the skilled person. Preferably, the cell population comprising T cells is a buffy coat, a preparation of peripheral blood mononuclear cells (PBMCs), ore a preparation of cells derived therefrom. Preferably, said cell population is comprised in a suitable liquid, such as a buffer or a nutrient medium.

The cell population is known or suspected to contain NK cells. As will be appreciated by the skilled person, NK cells are almost ubiquitously found in cell-containing samples from mammalian subjects, so in principle any sample of a subject or a preparation thereof will be at least suspected to comprise NK cells, unless special precautions were taken to exclude and/or remove NK cells. Methods of identifying NK cells in cell populations are well known in the art and include in particular determining morphology and/or surface markers as specified herein above.

The method for producing a preparation of T cells comprises step (i) contacting said cell population with a T cell-NK cell interaction inhibitor. The term “contacting” is understood by the skilled person; preferably, the term comprises bringing two compounds to be contacted into admixture such as to enable physical contact between said compounds. Preferably, contacting comprises maintaining said admixture for a time and under conditions allowing said interaction to occur and at least not being detrimental to the compounds interacting, i.e. in particular the T cells and the T cell-NK cell interaction inhibitor. The skilled person knows how to adjust appropriate conditions, in particular depending on the type of T cell-NK cell interaction inhibitor contacted. Step (i) may be performed concomitant to step (ii), may, however, also be performed before step (ii). In principle, in particular in case the fraction of NK cells in the cell preparation is expected to be low, step (i) may even be performed after step (ii), or at least after step (ii) was started. As will be appreciated, the relative timing of steps (i) and (ii) will be decided by the skilled person in view of the description herein depending e.g. on the relative fraction of NK cells expected, but also depending on the type T cell-NK cell interaction inhibitor to be contacted. E.g. in case the T cell-NK cell interaction inhibitor is a permanent inhibitor, step (i) may be performed before or concomitant to step (ii), while in the case of a transient inhibitor performing steps (i) and (ii) concomitantly, preferably for the whole duration of step (ii), may be preferable.

The method for producing a preparation of T cells comprises step (ii) expanding T cells. Methods for expanding T cells are known in the art and are described herein in the Examples. Preferably, expanding T cells comprises increasing the relative, preferably the absolute, number of T cells in a cell population. Thus, expanding T cells preferably comprises maintaining T cells under conditions allowing the T cells to proliferate. The duration of step (ii) will essentially be governed by the initial number of T cells, the growth rate of the T cells, and the desired number of T cells which shall be obtained.

The method for producing a preparation of T cells comprises step (iii) producing a preparation of T cells. Said step (iii) preferably is the result of performing steps (i) and (ii) as specified herein above. Thus, preferably, the preparation of T cells comprises T cells enriched in relative numbers and/or in absolute number compared to the cell population comprising T cells used in step (i). Also preferably, in the T cell preparation T cell-NK cell interaction is inhibited permanently and/or transiently. As specified herein above, the preparation of T cells may be enriched or consist of a subset of T cells, such as CD4+ T cells and/or tumor-specific T cells.

The present invention also relates to a preparation of T cells comprising a modification decreasing B7H6 activity.

The term “preparation of T cells” includes in principle any and all compositions of matter comprising living T cells, wherein the further components of said composition of matter are at least not detrimental to the survival of said T cells. Preferably, said decrease in B7H6 activity comprises decrease of B7H6 expression in the T cells in said population. Preferably, said modification is a temporary modification, e.g. used to allow the production of an activated preparation of T cells despite the presence of NK cells, e.g. in vitro. More preferably, the modification is a long-lasting modification, i.e. lasting over weeks or months, e.g. to allow the T cells to be active in vivo for a planned treatment period despite the presence of NK cells. Even more preferably, the modification is a permanent modification, e.g. to protect the preparation of T cells from effects of NK cells after administration in vivo. In accordance with the above, the preparation of T cells preferably has a decreased activity of B7H6 compared to a control population of T cells. Methods for measuring B7H6 activity are known in the art and are described herein in the Examples. Preferably, the B7H6 activity in the preparation of T cells is decreased to at most 75%, preferably at most 50%, more preferably at most 25%, most preferably at most 10% of a control population of T cells. Also preferably, the B7H6 activity in the preparation of T cells is below the detection limit, preferably of the assay described herein in the Examples. Also preferably, the T cell preparation comprises a T cell-NK cell interaction inhibitor as specified herein above and/or comprises a genetic modification causing a decrease in the expression of the gene encoding B7H6, preferably compared to a population of non-modified T cells. Also preferably, the modification is a knockout of the gene(s) encoding B7H6, preferably the NCR3LG1 gene, and/or comprises expression of a shRNA, a siRNA, a miRNA agent, an antisense oligonucleotide, and/or a ribozyme causing a decrease in expression of the gene(s) encoding B7H6. Thus, the T cells in the T cell preparation preferably are B7H6low or B7H6.

Preferably, the T cells in the T cell preparation are active and/or long-term persistent T cells Also preferably, the T cells in the T cell preparation are anti-cancer T cells, preferably anti-cancer antigen T cells, and/or are tumor infiltrating lymphocytes (TILs) or are derived from TILs. Preferably, the T cells in the T cell preparation express a chimeric antigen receptor (CAR) and/or a recombinant T cell receptor (rTCR). More preferably, the T cells in the T cell preparation persist and/or are active after administration to a subject for at least 2 weeks, preferably at least 4 weeks, more preferably for at least 8 weeks. Preferably, the half-life and/or the activity half-life of the T cells after administration to a subject is at least 2 weeks, preferably at least 4 weeks, more preferably at least 8 weeks.

Preferably, the T cell preparation is obtained or obtainable according to the method for producing a preparation of T cells, using a T cell-NK cell interaction inhibitor decreasing B7H6, as specified herein above.

Preferably, the aforesaid T cell preparation is obtained or obtainable according to the method for producing a preparation of T cells, using a T cell-NK cell interaction inhibitor decreasing B7H6 as specified herein above.

The present invention also relates to a preparation of T cells as specified herein above for use in medicine, in particular for use in treatment and/or prevention of cancer.

Treatment and prevention of cancer have been described herein above. Treatment and/or prevention of cancer may in particular comprise administration of a T cell preparation of the present invention and/or administration of a T cell-NK cell interaction inhibitor of the present invention. The T cell preparation in such case preferably comprises tumor-infiltrating T cells, CAR-comprising T cells and/or rTCR comprising T cells, wherein said T cells preferably are autologous T cells. However, in principle also allogenic T cells may be used.

Further, the present invention relates to a method for improving in vivo persistence and/or activity of T cells in a subject comprising contacting said subject with a T cell-NK cell interaction inhibitor of the present invention and/or with a preparation of T cells of the present invention, and, thereby, improving in vivo persistence and/or activity of T cells.

The present invention also relates to a use of a T cell-NK cell interaction inhibitor of the present invention and/or a preparation of T cells of the present invention for the manufacture of a medicament, preferably for improving persistence and/or activity of T cells.

Further, the present invention relates to a method of identifying a subject amenable to treatment with a T cell-NK cell interaction inhibitor, comprising
(a) determining expression of the NCR3LG1 gene in T cells of said subject, preferably in TILs of said subject,
(b) comparing the amount determined in step (a) to a reference, and
(c) based on the comparison in step (b), determining said subject to be amenable to said treatment or not.

The method of identifying a subject of the present invention, preferably, is an in vitro method. Moreover, it may comprise steps in addition to those explicitly mentioned above. Moreover, one or more of said steps may be assisted or performed by automated equipment.

Methods for determining expression of the NCR3LG1 gene in T cells of a subject are known in the art and include in particular mRNA expression analysis, including generalized methods such as next generation sequencing of cDNAs obtained from at least one T cell, as well as methods of determining the amount of B7H6 in and/or on the surface of a T cell, e.g. by immunological methods, such as immunoblot, ELISA, FACS, or in situ immunohistochemistry methods.

Suitable references are references with known known level of expression of the NCR3LG1 gene or values derived therefrom. Thus a reference may e.g. (i) be derived from T cells known to have an expression of the NCR3LG1 gene essentially corresponding to the average expression in a population of apparently healthy subjects, preferably of T cells thereof. A reference may, however, also (ii) be derived from T cells known to have a decreased expression of the NCR3LG1 gene. Preferably, a subject is identified to be amenable to treatment with a T cell-NK cell interaction inhibitor in case expression of the NCR3LG1 gene determined in step (a) is essentially as equal to or higher than in reference (i) and/or is higher than reference (ii). In accordance, a subject is identified to be not amenable to treatment with a T cell-NK cell interaction inhibitor in case expression of the NCR3LG1 gene determined in step (a) is essentially as high or lower than in reference (ii) and/or is lower than reference (i).

Moreover, the present invention relates to a method for identifying a T cell-NK cell interaction inhibitor, said method comprising
(A) contacting a cell population comprising T cells and NK cells with a candidate T cell-NK cell interaction inhibitor;
(B) determining T cell-NK cell interaction and/or T cell number; and
(C) based on the determining in step (B), identifying a T cell-NK cell interaction inhibitor.

The method of identifying a subject of the present invention, preferably, is an in vitro method. Moreover, it may comprise steps in addition to those explicitly mentioned above. Also, one or more of said steps may be assisted or performed by automated equipment; in particular, the method may be performed in a high-throughput setting.

The term “candidate T cell-NK cell interaction inhibitor” may, in principle, relate to any chemical compound for which the skilled person may assume that it could be a T cell-NK cell interaction inhibitor. As will be understood by the skilled person, such a presumption may in principle apply to any compound, in particular in the case of low molecular weight compounds. In the case of a high molecular weight compound, the presumption may be based on sequence similarity to the NCR3LG1 gene or a fragment thereof, or, in case the candidate compound is an antibody, on the fact that the antibody was generated using B7H6 or an NCR as an antigen.

Methods for determining T cell-NK cell interaction and/or T cell number are known in the art and are described herein in the Examples. Preferably, T cell-NK cell interaction and/or T cell number are determined in step (B) compared to a control cell population not contacted with the candidate T cell-NK cell interaction inhibitor.

In view of the description herein, the skilled person understands that in step (C) a T cell-NK cell interaction inhibitor is preferably identified if it is found in step (B) that T cell-NK cell interaction is decreased and/or that T cell number is higher compared to a control.

Further, the present invention also relates to a kit comprising a preparation of T cells according to the present invention and at least one T cell-NK cell interaction inhibitor according the present invention, preferably comprised in a housing.

The term “kit”, as used herein, refers to a collection of the aforementioned compounds, means or reagents which may or may not be packaged together. The components of the kit may be comprised by separate vials (i.e. as a kit of separate parts) or provided in a single vial, e.g. as a composition as specified herein above. The housing of the kit in an embodiment allows translocation of the compounds of the kit, in particular common translocation; thus, the housing may in particular be a transportable container comprising all specified components. Moreover, it is to be understood that the kit of the present invention may be used for practicing the methods referred to herein above. It is, in an embodiment, envisaged that all components are provided in a ready-to-use manner for practicing the methods referred to above. Further, the kit preferably contains instructions for carrying out said methods. The instructions can be provided by a user's manual on paper or in electronic form. For example, the manual may comprise instructions for interpreting the results obtained when carrying out the aforementioned methods using the kit. Preferably, the kit is adapted for use in a method of the present invention, more preferably is adapted to comprise all reagents required to perform said method or methods.

The present invention also relates to a device comprising a preparation of T cells according to the present invention.

The term “device”, as used herein relates to a system of means comprising at least the means operatively linked to each other as to allow administration of the compound or of the composition of the present invention. Preferred means for administering a preparation comprising T cells are well known in the art. How to link the means in an operating manner will depend on the type of means included into the device and on the kind of administration envisaged. Preferably, the means are comprised by a single device in such a case. Said device may accordingly include a delivery unit for the administration of the preparation and, optionally, a storage unit for storing said preparation until administration. However, it is also contemplated that the means of the current invention may appear as separate devices in such an embodiment and are, preferably, packaged together as a kit. The person skilled in the art will realize how to link the means without further ado. Preferred devices are those which can be applied without the particular knowledge of a specialized technician. In a preferred embodiment, the device is a syringe, more preferably with a needle, comprising the preparation of the invention. More preferably, the device is an intravenous infusion (IV) equipment comprising the preparation. Also preferably, the device is a tube or an endoscopic device comprising the preparation for flushing a site of administration, e.g. a heart, or further comprising a needle for topical application of the compound or composition, e.g. to a heart.

Furthermore, the present invention relates to a use of a T cell-NK cell interaction inhibitor for expanding T cells.

In view of the above, the following embodiments are particularly envisaged:
Embodiment 1: A T cell-NK cell interaction inhibitor for use in improving in vivo persistence and/or activity of T cells.
Embodiment 2: The T cell-NK cell interaction inhibitor for use of embodiment 1, wherein said T cell-NK cell interaction is a B7H6 mediated interaction.
Embodiment 3: The T cell-NK cell interaction inhibitor for use of embodiment 1 or 2, wherein said T cell-NK cell interaction is a natural cytotoxicity triggering receptor (NCR) mediated interaction, preferably wherein said NCR is NCR3, NCR2, or NCR1.
Embodiment 4: The T cell-NK cell interaction inhibitor for use of any one of embodiments 1 to 3, wherein said T cell-NK cell interaction inhibitor is an inhibitor of B7 homolog 6 (B7H6) mediated T cell-NK cell interaction.
Embodiment 5: The T cell-NK cell interaction inhibitor for use of any one of embodiments 1 to 4, wherein said T cell-NK cell interaction inhibitor decreases the activity of B7H6 on T cells, decreases the activity of at least one NCR on NK cells, and/or decreases interaction between B7H6 on T cells and at least one NCR on NK cells.
Embodiment 6: The T cell-NK cell interaction inhibitor for use of any one of embodiments 1 to 5, wherein said T cell-NK cell interaction inhibitor is an inhibitor of B7H6 interaction with NCR3.
Embodiment 7: The T cell-NK cell interaction inhibitor for use of any one of embodiments 1 to 6, wherein said T cell-NK cell interaction inhibitor is an indirect T cell-NK cell interaction inhibitor decreasing the amount of B7H6 in a T cell or of an NCR, preferably NCR3, in an NK cell.
Embodiment 8: The T cell-NK cell interaction inhibitor for use of embodiment 7, wherein said indirect T cell-NK cell interaction inhibitor is selected from the list consisting of an shRNA, a siRNA, a miRNA agent, an antisense oligonucleotide, a ribozyme, and at least one CRISPR/Cas oligonucleotide, preferably a pair of CRISPR/Cas oligonucleotides.
Embodiment 9: The T cell-NK cell interaction inhibitor for use of embodiment 7 or 8, wherein said T cell-NK cell interaction inhibitor is an siRNA comprising the sequence, or is a CRISPR/Cas oligonucleotide comprising the sequence of any one of SEQ ID NOs: 1 to 10.
Embodiment 10: The T cell-NK cell interaction inhibitor for use of any one of embodiments 1 to 9, wherein said T cell-NK cell interaction inhibitor is an expression construct causing expression of a shRNA, a siRNA, a miRNA agent, an antisense oligonucleotide, a ribozyme, and at least one CRISPR/Cas oligonucleotide decreasing the amount of B7H6 in a T cell or of an NCR, preferably NCR3, in an NK cell when expressed in a T cell and/or in an NK cell.
Embodiment 11: The T cell-NK cell interaction inhibitor for use of any one of embodiments 1 to 6, wherein said T cell-NK cell interaction inhibitor is a direct T cell-NK cell interaction inhibitor inhibiting interaction between B7H6 and at least one NCR.
Embodiment 12: The T cell-NK cell interaction inhibitor for use of any one of embodiments 1 to 6 and 11, wherein said direct T cell-NK cell interaction inhibitor is a compound binding to the extracellular domain of B7H6 or of NCR3 and at least partially inhibiting B7H6-NCR3 interaction.
Embodiment 13: The T cell-NK cell interaction inhibitor for use of any one of embodiments 1 to 6, 11, and 12, wherein said direct T cell-NK cell interaction inhibitor is a small molecule inhibitor, an inhibitor polypeptide, an inhibitor polynucleotide, or a non-polypeptide non-polynucleotide inhibitor macromolecule.
Embodiment 14: The T cell-NK cell interaction inhibitor for use of any one of embodiments 1 to 6 and 11 to 13, wherein said direct T cell-NK cell interaction inhibitor is a molecule from the immunoglobulin family of polypeptides or a fragment thereof, an aptamer, an anticalin, or a Designed Ankyrin Repeat Protein (DARPin).
Embodiment 15: The T cell-NK cell interaction inhibitor for use of any one of embodiments 1 to 6 and 11 to 14, wherein said T cell-NK cell interaction inhibitor comprises an antibody or a fragment thereof.
Embodiment 16: The T cell-NK cell interaction inhibitor for use of any one of embodiments 1 to 6 and 11 to 15, wherein said antibody is an NCR3 blocking antibody or a B7H6 blocking antibody, preferably is a monoclonal antibody reactive against recombinant human NCR3.
Embodiment 17: The T cell-NK cell interaction inhibitor for use of any one of embodiments 1 to 16, wherein improving in vivo persistence and/or activity of T cells is improving in vivo persistence and/or activity of anti-tumor T cells.
Embodiment 18: The T cell-NK cell interaction inhibitor for use of any one of embodiments 1 to 17 wherein said improving in vivo persistence and/or activity of T cells comprises treating and/or preventing cancer.
Embodiment 19: The T cell-NK cell interaction inhibitor for use of any one of embodiments 1 to 18, wherein said improving in vivo persistence and/or activity of T cells, preferably said treating and/or preventing cancer, comprises administration of in vitro expanded T cells.
Embodiment 20: The T cell-NK cell interaction inhibitor for use of any one of embodiments 1 to 19, wherein said T cells are tumor infiltrating lymphocytes (TILs) and/or T cells expressing a chimeric antigen receptor (CAR) and/or T cells expressing a recombinant T cell receptor (rTCR).
Embodiment 21: The T cell-NK cell interaction inhibitor for use of any one of embodiments 18 to 20, wherein said cancer is a solid cancer and wherein said T cell-NK cell interaction inhibitor is administered intratumorally.
Embodiment 22: The T cell-NK cell interaction inhibitor for use of any one of embodiments 1 to 21, wherein said T cell-NK cell interaction causes T cell lysis.
Embodiment 23: The T cell-NK cell interaction inhibitor for use of any one of embodiments 1 to 22, wherein said T cell is a B7H6 positive T cell.
Embodiment 24: The T cell-NK cell interaction inhibitor for use of any one of embodiments 1 to 23, wherein said T cell is a B7H6+/CD4+ or a B7H6+/CD8+ T cell.
Embodiment 25: The T cell-NK cell interaction inhibitor for use of any one of embodiments 18 to 24, wherein said cancer is a glioblastoma, a multiple myeloma, or is colorectal cancer.
Embodiment 26: A method for producing a preparation of T cells from a cell population comprising T cells and known or suspected to comprise NK cells, said method comprising (i) contacting said cell population with a T cell-NK cell interaction inhibitor, (ii) expanding T cells, and (iii) thereby, producing a preparation of T cells.
Embodiment 27: The method of embodiment 26, wherein said method is an in vitro method.
Embodiment 28: A preparation of T cells comprising a modification decreasing B7H6 activity.
Embodiment 29: The preparation of embodiment 28, wherein said modification is a genetic modification.
Embodiment 30: The preparation of embodiment 28 or 29, wherein said genetic modification causes a decrease in the expression of the gene encoding B7H6, preferably compared to a population of non-modified T cells.
Embodiment 31: The preparation of any one of embodiments 28 to 30, wherein said modification is a knockout of the gene(s) encoding B7H6, preferably the NCR3LG1 gene, and/or comprises expression of a shRNA, a siRNA, a miRNA agent, an antisense oligonucleotide, and/or a ribozyme causing a decrease in expression of the gene(s) encoding B7H6
Embodiment 32: A preparation of T cells obtained or obtainable according to the method of embodiment 26 or 27.
Embodiment 33: The preparation of any one of embodiments 28 to 32, wherein said T cells are active and/or long-term persistent T cells.
Embodiment 34: The preparation of any one of embodiments 28 to 33, wherein said T cells are anti-cancer T cells, preferably anti-cancer antigen T cells.
Embodiment 35: The preparation of any one of embodiments 28 to 34, wherein said T cells are tumor infiltrating lymphocytes (TILs) or are derived from TILs.
Embodiment 36: The preparation of any one of embodiments 28 to 35, wherein said T cells express a chimeric antigen receptor (CAR) and/or a recombinant T cell receptor (rTCR).
Embodiment 37: The preparation of any one of embodiments 28 to 36, wherein said T cells are B7H6^{low} or B7H6⁻.
Embodiment 38: The preparation of any one of embodiments 28 to 37, wherein said T cells persist and/or are active after administration to a subject for at least 2 weeks, preferably at least 4 weeks, more preferably for at least 8 weeks.
Embodiment 39: The preparation of any one of embodiments 28 to 38, wherein the half-life and/or the activity half-life of the T cells after administration to a subject is at least 2 weeks, preferably at least 4 weeks, more preferably at least 8 weeks.
Embodiment 40: The preparation of any one of embodiments 28 to 39 for use in medicine.
Embodiment 41: The preparation of any one of embodiments 28 to 39 for use in treatment and/or prevention of cancer.
Embodiment 42: A method for improving in vivo persistence and/or activity of T cells in a subject comprising contacting said subject with a T cell-NK cell interaction inhibitor as specified in any one of embodiments 1 to 25 and/or with a preparation of T cells od any one of embodiments 28 to 39, and, thereby, improving in vivo persistence and/or activity of T cells.
Embodiment 43: The method of embodiment 42, wherein said improving in vivo persistence and/or activity of T cells is comprised in treating and/or preventing cancer.
Embodiment 44: The method of embodiment 42 or 43, wherein said subject is known or suspected to suffer from or is at risk of developing cancer.
Embodiment 45: Use of a T cell-NK cell interaction inhibitor as specified in any one of embodiments 1 to 25 and/or a preparation of T cells of any one of embodiments 28 to 39 for the manufacture of a medicament, preferably for improving persistence and/or activity of T cells.
Embodiment 46: A method of identifying a subject amenable to treatment with a T cell-NK cell interaction inhibitor, comprising
(a) determining expression of the NCR3LG1 gene in T cells of said subject, preferably in TILs of said subject,
(b) comparing the amount determined in step (a) to a reference, and
(c) based on the comparison in step (b), determining said subject to be amenable to said treatment or not.
Embodiment 47: A method for identifying a T cell-NK cell interaction inhibitor, said method comprising
(A) contacting a cell population comprising T cells and NK cells with a candidate T cell-NK cell interaction inhibitor;
(B) determining T cell-NK cell interaction and/or T cell number; and
(C) based on the determining in step (B), identifying a T cell-NK cell interaction inhibitor.
Embodiment 48: The method of embodiment 47, wherein step (A) additionally comprises stimulating said T cells.
Embodiment 49: A kit comprising a preparation of any one of embodiments 28 to 39 and at least one T cell-NK cell interaction inhibitor according to any one of embodiments 1 to 25, preferably comprised in a housing.
Embodiment 50: A device comprising a preparation of any one of embodiments 28 to 39.
Embodiment 51: Use of a T cell-NK cell interaction inhibitor for expanding T cells.
Embodiment 52: The use of embodiment 51, wherein said use is an in vitro use.
Embodiment 53: The use of embodiment 51 or 52, wherein said T cells are expanded from a cell population known or suspected to comprise NK cells.
Embodiment 54: The subject matter of any of the preceding embodiments, wherein said NK cell is a CD3 CD56⁺ NK cell.

All references cited in this specification are herewith incorporated by reference with respect to their entire disclosure content and the disclosure content specifically mentioned in this specification.

### FIGURE LEGENDS

FIG. 1: A. Co-culture of activated human T cells (T cells only and activated T cells+autologous NK cells (T+NK cells) over a time span of 8 h. Quantification of absolute T cells in co-culture by flow cytometry, n=3 healthy donors. Mean±SEM shown. Statistical significance was determined by unpaired two-tailed t test at indicated time points; relative T cell counts normalized to baseline (To) are indicated. B. ⁵¹Cr-release-assay of non-activated and activated human T cells co-cultured with autologous NK cells after 8 h; n=3 healthy donors. Mean±SEM shown. Two-way ANOVA with Sidak's multiple comparison test. C. C57BL/6J mice challenged with syngeneic subcutaneous tumors and treated with NK cell depleting antibody or isotype control in vivo. Tumor volume measurements of syngeneic subcutaneous tumors shown; n=9 per group. Mean±SEM shown. Statistical significance was determined by unpaired two-tailed t test at indicated time point. D. C57BL/6J mice challenged with syngeneic subcutaneous tumors and treated with NK cell depleting antibody or isotype control in vivo. Fraction of activated tumor-infiltrating T cells among CD8⁺ T cells on day 10 after tumor inoculation quantified by flow cytometry. n=7 mice per group. Mean±SEM shown. Statistical significance was determined by unpaired two-tailed t test.

FIG. 2: A. Co-culture of activated T cells with autologous peripheral NK cells with NKp-blocking antibodies or isotype control, analyzed using flow cytometry after 72 h. Mean±SEM shown. Statistical significance was determined by two-tailed paired t test; relative T cell counts normalized to baseline (T₀) are indicated. B. Immunofluorescence staining quantification of B7H6 on activated and non-activated human T cells after cytospin immobilization; n=4 healthy donors. Mean±SEM shown. Statistical significance was determined by two-tailed paired t test. C. Representative expression of B7H6 on primary T cells electroporated with SpCas9 RNPs containing B7H6-targeting or non-targeting sgRNAs, 10 days after electroporation and after 3 days of activation. Mean±SEM shown. Statistical significance was determined by unpaired two-tailed t test at indicated time points. D. Co-culture of activated human T cells with autologous NK cells, analyzed after 24 h by flow cytometry. NK Cells were isolated from n=5 healthy donors. Mean±SEM shown. Statistical significance was determined by two-tailed paired t test; relative T cell counts normalized to baseline (To) are indicated.

FIG. 3: (A) Experimental overview. B7H6−/− and B7H6+/+ Jurkat cells were transduced with a CD19.CD28.4-1BBzeta-CAR construct and injected in an 1:1 ratio into NSG mice together with NK cells from n=6 healthy donors. (B) Ratio of B7H6+/+ and B7H6−/− Jurkat CAR-T cells in the peripheral blood of NSG mice 2 days after transfer. (C) Expansion of CAR-T cells from day 9 to day 15 after tumor injection in peripheral blood of Nalm6-bearing NSG mice. (D) Total flux of IVIS optical imaging signal in Nalm6 Luciferase-expressing tumor bearing mice 15 days after adoptive transfer. The signal intensity positively correlates with the cumulative tumor mass in these tumor-bearing mice. (E) Kaplan-Meier-survival statistic of Nalm6-bearing mice after adoptive transfer of CAR-T cells, control T cells, and autologous NK cells.

FIG. 4: (A) B7H6-positive CAR-T cells in the peripheral blood of CD19 CAR-T cell treated patients at two different time points after CAR-T cell transfer. (B) Percentages of B7H6+ CAR-T cells in patient peripheral blood post infusion overlaid with peripheral blood CD56+ NK cell frequency.

FIG. 5: (A) Violin plot for the quantification of B7H6⁺CD3⁺ cells in clinical responder (R) and non-responder (NR) in a cohort of patients with relapsed esophageal squamous cell carcinoma (ESCC) that were treated with nivolumab (anti-PD-1) in a safety run-in phase and continued with nivolumab and ipilimumab (anti-CTLA-4) until relapse. The abundance of immune cells was assessed by immunofluorescence staining in endoscopic post-treatment biopsy samples (B, C) Violin plot for the quantification of B7H6⁺CD4⁺ (B) and B7H6⁺CD8⁺ (C) cells in clinical responder (R) and non-responder (NR) patients. (D) Ratio of NKp46+NK cells to CD3+ T cells in clinical responder (R) and non-responder (NR) patients.

FIG. 6: (A) Log2 (tpm+1) values from n=184 bulk RNA-sequencing samples for NCR3LG1 are shown for resting human T cells cells as well as cells collected at 7 subsequent time points after anti-CD3/CD28 stimulation. Horizontal lines within violin plots denote median expression values. Dynamic expression was modeled as 2^{nd} order polynomial with time as predictor variable and accounting for donor as random effect. The shown p-value was obtained from a likelihood ratio test. (B) Quantification of B7H6 surface protein expression in CD8⁺ and CD4⁺ T cells, n=4 healthy donors at indicated time points after anti-CD3/CD28 stimulation. (C) Bar chart depicting frequency of NCR3LG1⁺ T cells split by disease entity. MS, multiple sclerosis; IBD, inflammatory bowel disease; HBV, hepatitis B virus infection; BC, bladder cancer; BCL, B cell lymphoma; ESCA, esophageal cancer; OV, ovarian cancer; MM, multiple myeloma; PACA, pancreatic cancer; RC, renal carcinoma; THCA, thyroid carcinoma; UCEC, uterine corpus endometrial carcinoma.

FIG. 7: (A) Quantification of shed B7H6 in tumor cell supernatant over time measured by ELISA in 11 different tumor cell lines (B) Quantification of shed B7H6 in tumor cell supernatant over time measured by ELISA in 3 patient-derived primary tumor organoids. (C) Killing of primary human T cells by autologous human NK cells in the presence of increasing concentrations of tumor cell (Mel624) supernatant.

The following Examples shall merely illustrate the invention. They shall not be construed, whatsoever, to limit the scope of the invention.

### Example 1: CRISPR-Cas9 Knockout in Primary T Cells

Primary T cells were isolated and incubated in T Cell Expansion Medium supplemented with iU IL2 and L-Glutamine. T cells were activated for 72 hours using Human CD3/CD28/CD2 T Cell Activator beads (Stemcell). 1.5×10⁵ T cells were resuspended in T resuspension buffer (Thermo Fisher Scientific), mixed with the spCas9/gRNA ribonucleoprotein (RNP) complexes complexes at a 1:1 ratio and electroporated using the following settings on a Neon Transfection System and 10 μl tips: EP 1400 V, Pulse width 30 ms, number of pulses 1. For data shown in FIG. 2, gRNA sequences with SEQ ID NOs: 3 and 9, respectively (Table 1), were used for RNPs; function of SEQ ID NOs: 1 to 10 in knockout was experimentally verified. Subsequently, T cells were incubated for 48 hours in T Cell Expansion Medium supplemented with 130 iU IL2, 2 mM L-Glutamine, 50 μg/mL gentamicin, 20 ng/ml IL-15 and IL-7. Knockout efficiency was checked via flow cytometry 10 days after electroporation after 72 hours of stimulation with CD3/CD28/CD2 T Cell Activator.

### Example 2: CRISPR-Cas9 Knockout in CAR-T Cells

The B7H6 knockout was performed as described above. Subsequently, edited T cells were incubated for 2 days and transduced with the RVSFG.CD19.CD28.4-1BBzeta retroviral vector. Briefly, virus was plated on a 24-well non-tissue culture-treated plate, previously coated with 7 μg/ml RetroNectin and centrifuged for 1 h @ 4000 RPM. T cells were added, centrifuged for 1 h and incubated for 5 h. CAR-T cells were collected on day 5 for further analysis.

### Example 3: NK Cell Depletion by Antibody Treatment In Vivo

1×10⁵ GL261-SIINFEKI tumor cells were diluted in 2 μl sterile PBS (Sigma-Aldrich) and stereotactically implanted into the right hemisphere of 7-8 weeks old male C57BL/6J mice (coordinates: 2 mm right lateral of the bregma and 1 mm anterior to the coronal suture with an injection depth of 3 mm below the dural surface) using a 10 μl Hamilton micro-syringe driven by a fine step stereotactic device (Stoelting). Tumor growth was measured by MR imaging. 2×10⁵ MC38-Ova cells in 100 μl PBS were mixed 1:1 with matrigel and were injected into the shaved right flank of 7-8 weeks old male C57BL/6J mice. Tumor growth was monitored every two to three days with an electronic caliper and measuring length×width× depth. NK cells were depleted using 200 μg InVivoMAb anti-mouse NK1.1 (Bioxcell). Mice received intraperitoneal injections with 200 μg antibody 48 and 24 hours before tumor inoculation and consecutively every four to five days. InVivoMAb mouse IgG2a isotype control, unknown specificity (Bioxcell) was used as isotype control.

### Example 4 In Vitro T Cell Killing by Autologous NK Cells

Isolated human T cells and NK were incubated in indicated ratios. T cells were activated with human transact beads (Miltenyi) or ImmunoCult™ Human CD3/CD28/CD2 T Cell Activator (StemcellTechnologies) and IL-2 and labeled with 5 μM CFSE. NKp blocking antibodies or isotypes were used at 5 μg/ml.

### Example 5: Results

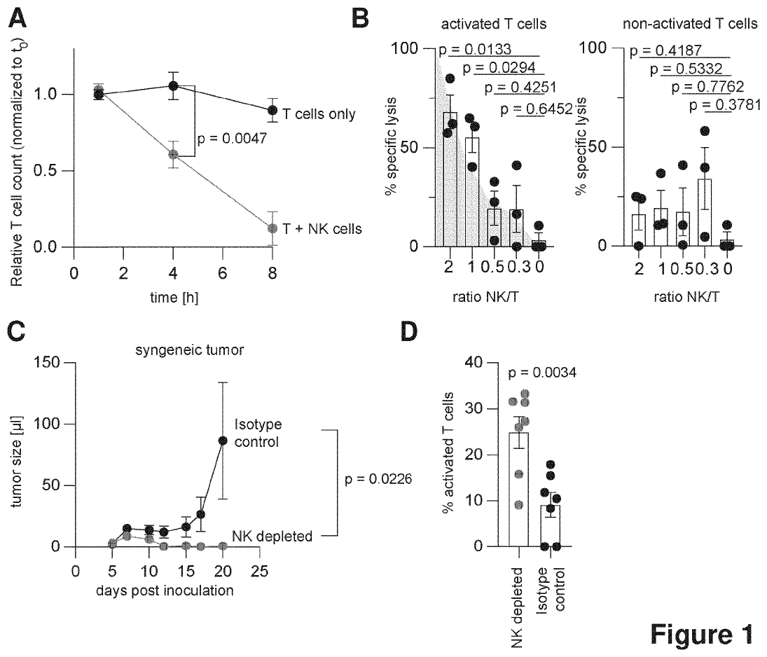

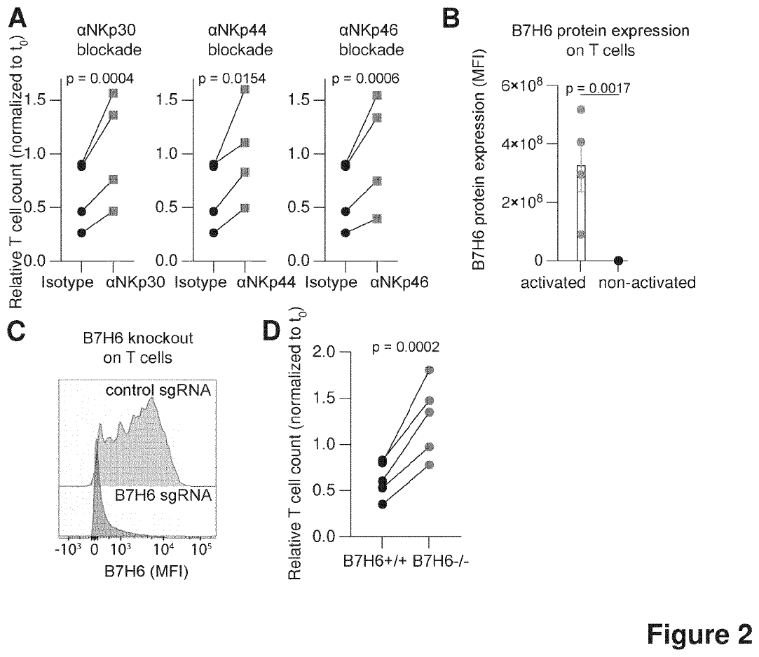

Human NK cells lyse autologous activated T cells (FIG. 1A). This lysis is enhanced with increasing NK/T cell ratio and specific to activated T cells as opposed to no-activated T cells (FIG. 1B). Antibody-mediated depletion of NK cells in an immunocompetent mouse model of subcutaneous cancer results in tumor rejection (FIG. 1C). This is accompanied by a relative increase in activated T cells in the tumor microenvironment of NK cell-depleted animals (FIG. 1D). Antibody-mediated blockade of NKp30, NKp44 and NKp46 on the surface of human NK cells results in reduced lysis of human activated T cells by autologous NK cells (FIG. 2A). Activated human T cells express B7H6 protein on the cell surface (FIG. 2B) Surface expression of B7H6 protein on human T cells can be reduced by knockout of the NCR3LG1 gene (FIG. 2C). Knockout of the human NCR3LG1 gene in human T cells results in reduced lysis of human activated T cells by autologous NK cells (FIG. 2D).

### Example 6: Further Results

Time-resolved deep bulk-RNA-sequencing of anti-CD3/CD28-stimulated peripheral blood-derived T cells from n=24 human donors revealed that NCR3LG1 transcript expression changes dynamically upon stimulation and that B7H6 surface protein recognition by NK cells initiates ‘fratricide’ as early as 8 hours post T cell stimulation (FIGS. 1 B and 6 A,B).

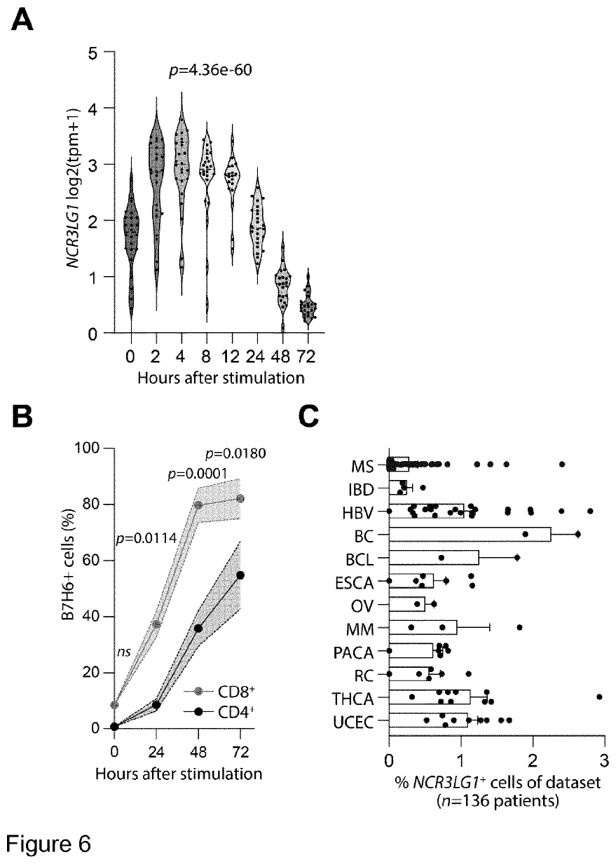

By analyzing transcriptomic profiles of 313,303 CD8+ and 283.930 CD4+ T cells in datasets of multiple sclerosis (MS, n=62 patients), inflammatory bowel disease (IBD, n=4 patients), hepatitis B virus infection (HBV, n=23 patients) and 9 different cancer entities (n=47 patients), we found a conserved B7H6+ T cell population that is prevalent in the tissue and blood of most individuals at 1-5% frequency (FIGS. 2B and 6C).

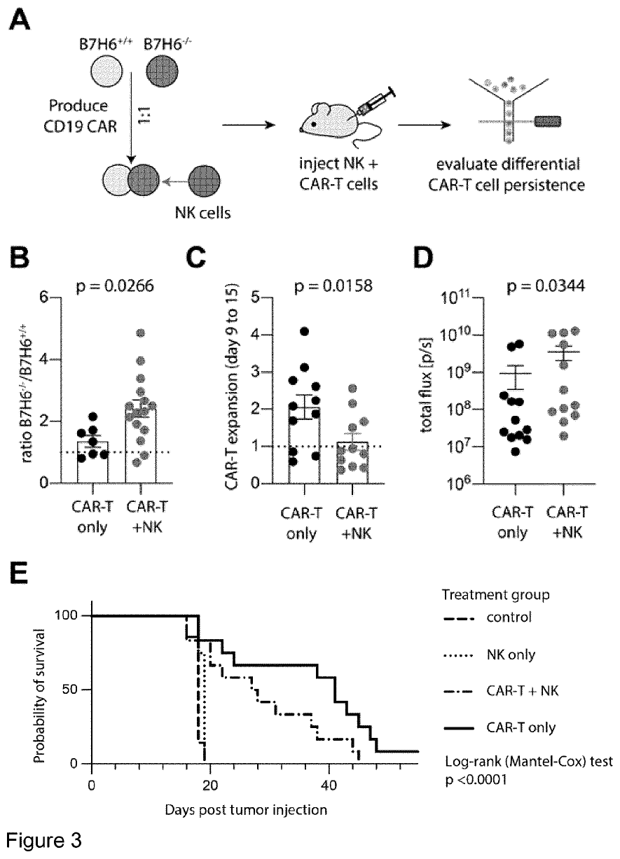

It was further determined whether the B7H6-NKp30 axis is a yet unknown, but relevant factor in limiting CAR T cell persistence: when B7H6−/− and B7H6+/+ T cells were transduced with a CAR construct (CD19.CAR T cells) and injected in an 1:1 ratio into NSG mice together with a mixture from NK cells from healthy donors (FIG. 3A), it was found that the additional presence of NK cells caused a massive depletion of B7H6+/+ CAR T cells cells in vivo (FIG. 3B), confirming the observation that B7H6 expression causes NK cells to lyse activated T cells. Co-injection of NK cells further lead to decreased CAR T cell expansion (FIG. 3C), increased tumor burden (FIG. 3D), and resulted in significantly decreased survival in leukemia-bearing mice treated with CD19-recognizing CAR-T cells (FIG. 3E), while CAR T cells with genetic ablation of NCR3LG1 (B7H6) were significantly more persistent in vivo (FIG. 3B).

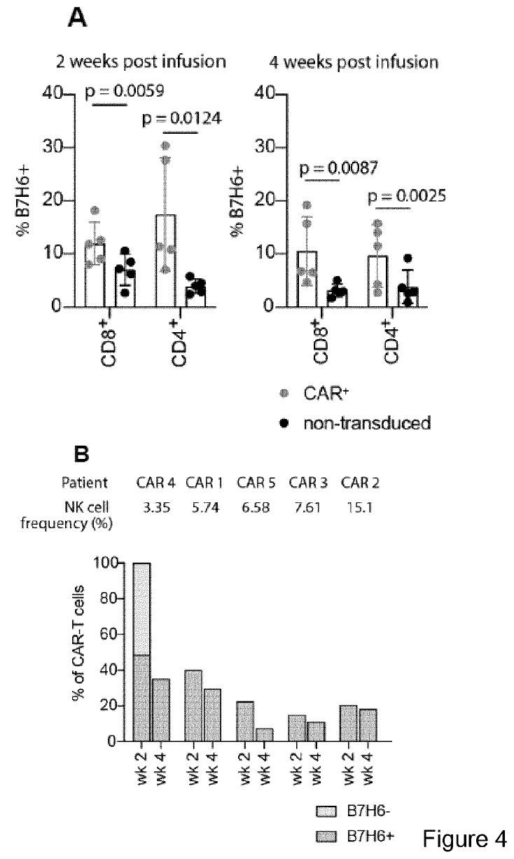

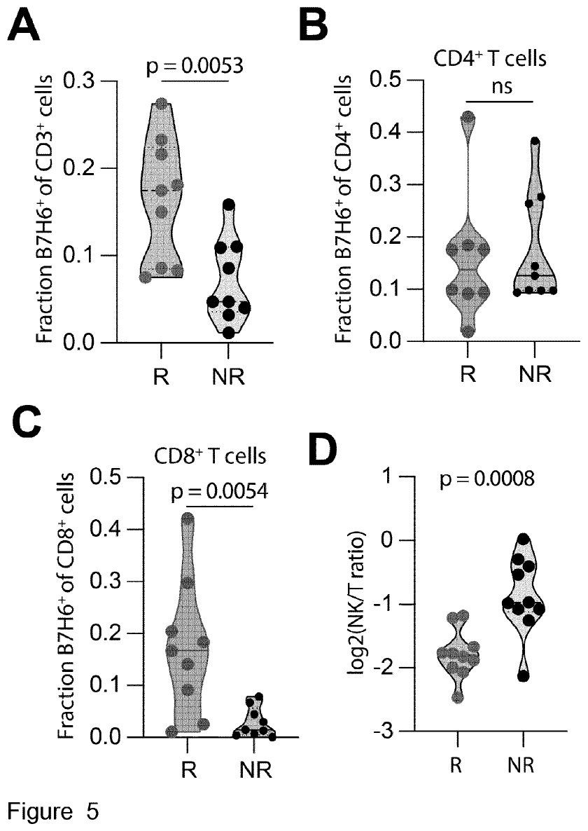

Similarly, the longitudinal dynamics of B7H6 protein expression after adoptive CAR-T cell transfer in human patients was studied. We analyzed peripheral blood from patients that have received CD19-targeting CAR-T cell therapy at two timepoints post infusion by flow cytometry using a B7H6-recognizing antibody (clone 1A5). When the time course of CAR T cell persistence was analyzed in peripheral blood, it was found that the number of B7H6+ T cells decreased over time (FIG. 4A). Notably, the degree of decrease correlated with the frequency of NK cells in the respective patients (FIG. 4 B).

Given the physiological coupling T cell activation and B7H6 expression, any T cell-directed immunotherapy regimen might trigger the B7H6-NKp30-cytolysis mechanism. We therefore analyzed tissue of patients that were treated with nivolumab and ipilimumab (NCT03416244) by immunofluorescence staining and correlated our findings with clinical endpoints in these patients (clinical response and progression-free survival).

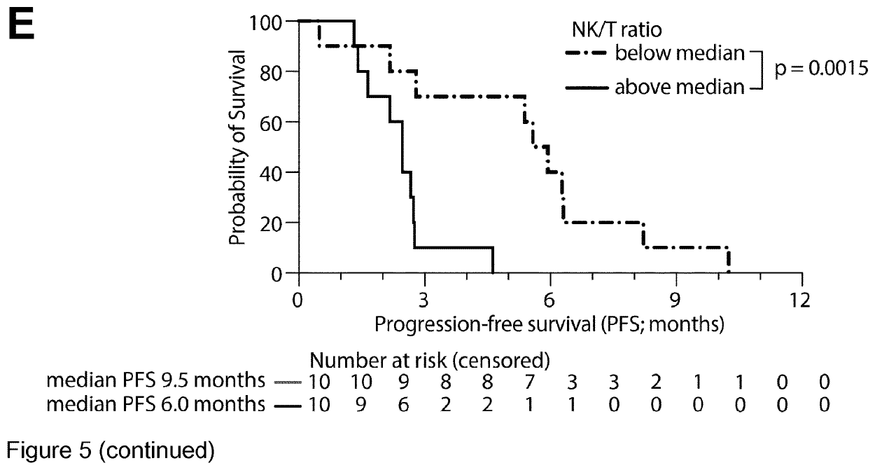

In contrast to the previous notion of largely tumor-targeting NK cell function, we observed an increased NK cell abundance in patients that failed to respond to treatment. Notably, we found both B7H6+CD8+ and CD4+ T cells in situ (FIG. 5A-C) and that low CD8+B7H6+ T cell abundance (FIG. 5C) and a high NK/T ratio (FIG. 5D) was associated with impaired clinical response and reduced progression-free survival (PFS), even in initial clinical responders (FIG. 5C, FIG. 5E).

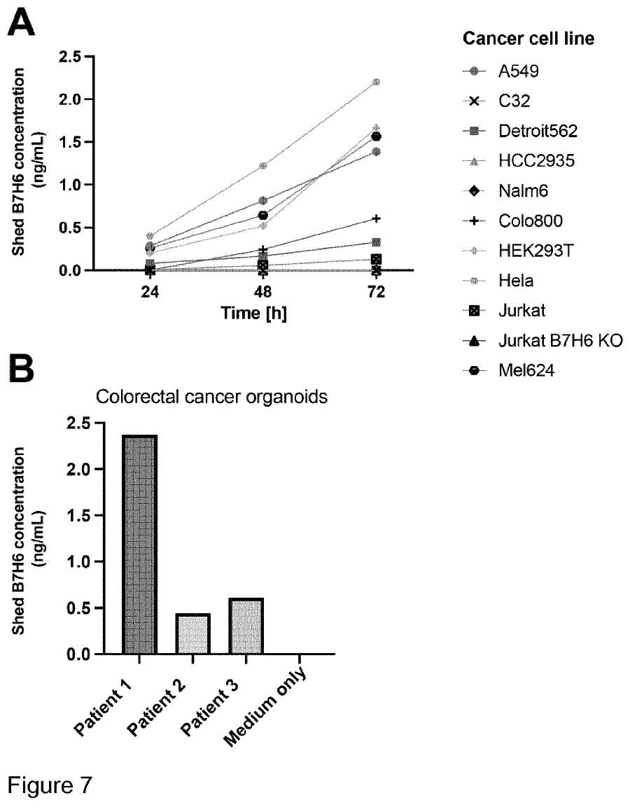

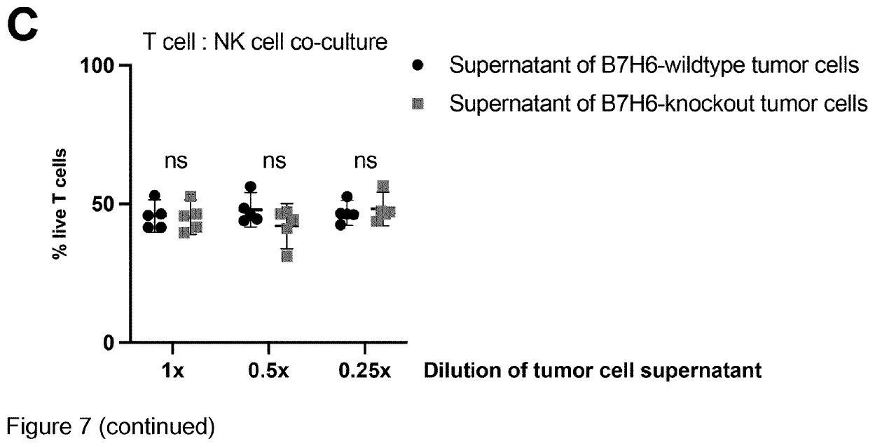

Shed B7H6 by tumor cells could potentially inhibit or trigger NK cell responses against T cells. Human cancer cell lines demonstrated heterogenous concentration of B7H6 (FIG. 7A). Similar results were obtained for primary tumor organoids derived from colorectal carcinoma patients (FIG. 7B). Importantly, killing of T cells by autologous NK cells was not affected when exposed to supernatant of tumor cells shedding high amounts of B7H6 (Mel624), indicating that NK cell killing of T cells is independent of shed B7H6 in the tumor microenvironment (FIG. 7C).

Together, our data demonstrate a mechanism that couples T cell expansion to eventual clearance by NK cells. We show that NK cell surveillance via B7H6 counteracts anti-tumor activity of CAR-T cells in vivo and that genetic or antibody-mediated inhibition of B7H6 enhances T cell persistence.

TABLE 1
CRISPR/Cas oligonucleotides: sgRNA
partial sequences hybridizing to the
NCR3LG1 coding sequence

| | SEQ ID |
|---|---|
| Sequence | NO: |
| AATCAGGAGCGCCGCGCACG | 1 |
| GGCGCTGACGACCGAAGGTA | 2 |
| AGCCGCGGCGATGACGTGGA | 3 |
| CGCTGACGACCGAAGGTAGG | 4 |
| CTGTGGGCGCTGACGACCGA | 5 |
| AGAATCAGGAGCGCCGCGCA | 6 |
| GGCGCTGACGACCGAAGGTA | 7 |
| AGCCGCGGCGATGACGTGGA | 8 |
| GGGTGACCACCACCTCACAT | 9 |
| AACTCCTCTCAGGAAGACCC | 10 |

### NON-STANDARD LITERATURE CITED

Bezman et al., Nat. Immunol. (2012) doi: 10.1038/ni.2395
Cózar et al., Cancer Discovery (2021) doi: 10.1158/2159-8290.CD-20-0655
Gross et al., Frontiers in Immunology (2016) doi: 10.3389/fimmu.2016.00606
Lu et al., Immunity (2007) doi: 10.1016/j.immuni.2007.03.017
Morvan & Lanier, Nature Reviews Cancer (2016) doi: 10.1038/nrc.2015.5
Orange, Journal of Allergy and Clinical Immunology (2013) doi: 10.1016/j.jaci.2013.07.020
Pahl & Cerwenka, Immunobiology (2017) doi: 10.1016/j.imbio.2015.07.012
Waggoner, et al., Nature (2012) doi: 10.1038/nature10624

1. A method for improving in vivo persistence and/or activity of T cells, the method comprising administering to a subject in need thereof a T cell-NK cell interaction inhibitor.

2. The method of claim 1, wherein said T cell-NK cell interaction inhibitor is an inhibitor of B7 homolog 6 (B7H6) mediated T cell-NK cell interaction.

3. The method of claim 1, wherein said T cell-NK cell interaction inhibitor is an indirect T cell-NK cell interaction inhibitor decreasing the amount of B7H6 in a T cell or of an NCR in an NK cell.

4. The method of claim 1, wherein said T cell-NK cell interaction inhibitor is an indirect T cell-NK cell interaction inhibitor decreasing the amount of B7H6 in a T cell.

5. The method of claim 1, wherein said indirect T cell-NK cell interaction inhibitor is selected from the group consisting of an shRNA, a siRNA, a miRNA agent, an antisense oligonucleotide, a ribozyme, and at least one CRISPR/Cas oligonucleotide.

6. The method of claim 5, wherein said T cell-NK cell interaction inhibitor is a CRISPR/Cas oligonucleotide comprising the sequence of any one of SEQ ID NOs: 1 to 10.

7. The method of claim 1, wherein said T cell-NK cell interaction inhibitor is a direct T cell-NK cell interaction inhibitor inhibiting interaction between B7H6 and at least one NCR.

8. The method of claim 7, wherein said direct T cell-NK cell interaction inhibitor is an inhibitor polypeptide, an inhibitor polynucleotide, or a non-polypeptide non-polynucleotide inhibitor macromolecule.

9. The method of claim 7, wherein said T cell-NK cell interaction inhibitor comprises an antibody or a fragment thereof.

10. The method of claim 7, wherein said antibody is an NCR3 blocking antibody or a B7H6 blocking antibody.

11. The method of claim 1, wherein said improving in vivo persistence and/or activity of T cells comprises treating and/or preventing cancer.

12. The method of claim 11, wherein said cancer is a glioblastoma, a multiple myeloma, or is colorectal cancer.

13. The method of claim 1, wherein said T cells are tumor infiltrating lymphocytes and/or T cells expressing a chimeric antigen receptor and/or T cells expressing a recombinant T cell receptor.

14. The method of claim 1, wherein said T cell is a B7H6 positive T cell.

15. A method for producing a preparation of T cells from a cell population comprising T cells and known or suspected to comprise NK cells, said method comprising (i) contacting said cell population with a T cell-NK cell interaction inhibitor, (ii) expanding T cells, and (iii) thereby, producing a preparation of T cells.

16. A method of identifying a subject amenable to treatment with a T cell-NK cell interaction inhibitor, said method comprising
(a) determining expression of the NCR3LG1 gene in T cells of said subject;
(b) comparing the amount determined in step (a) to a reference; and
(c) based on the comparison in step (b), determining said subject to be amenable to said treatment or not.

17. A method for identifying a T cell-NK cell interaction inhibitor, said method comprising
(A) contacting a cell population comprising T cells and NK cells with a candidate T cell-NK cell interaction inhibitor;
(B) determining T cell-NK cell interaction and/or T cell number; and
(C) based on the determining in step (B), identifying a T cell-NK cell interaction inhibitor.

18. (canceled)

19. The method of claim 7, wherein said T cell-NK cell interaction inhibitor is a direct T cell-NK cell interaction inhibitor inhibiting interaction between B7H6 and at least one NCR, wherein said direct T cell-NK cell interaction inhibitor is a compound binding to the extracellular domain of B7H6 or of NCR3 and at least partially inhibiting B7H6-NCR3 interaction.

20. The method of claim 10, wherein said antibody is a monoclonal antibody reactive against recombinant human NCR3.
